(19) United States
(12) Patent Application Publication
(10) Pub. No.: US 2023/0013823 A1
(43) Pub. Date: Jan. 19, 2023

(54) INHIBITORS OF HUMAN IMMUNODEFICIENCY VIRUS REPLICATION

(71) Applicant: VIV HEALTHCARE UK (No. 5) LIMITED, Brentford Middlesex (GB)

(72) Inventors: Michael S. BOWSHER, Wallingford, CT (US); Eric P. GILLIS, Branford, CT (US); B. Narasimhulu NAIDU, Branford, CT (US); Kyle E PARCELLA, Branford, CT (US); Manoj PATEL, Branford, CT (US)

(21) Appl. No.: 17/763,161

(22) PCT Filed: Oct. 2, 2020
(86) PCT No.: PCT/IB2020/059274

§ 371 Date: Mar. 23, 2022

Related U.S. Application Data
(60) Provisional application No. 62/910,684, filed on Oct. 4, 2019.

(51) Int. Cl. C07D 403/14; C07D 417/14; C07D 413/14

(52) U.S. Cl. CPC C07D 403/14; C07D 417/14; C07D 413/14

(57) ABSTRACT

Compounds of Formula I, including pharmaceutically acceptable salts thereof, and compositions and methods for treating human immunodeficiency virus (HIV) infection are set forth:

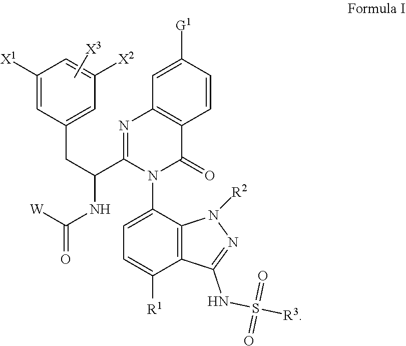

### FIELD OF THE INVENTION

The invention relates to compounds, compositions, and methods for the treatment of human immunodeficiency virus (HIV) infection. More particularly, the invention provides novel Capsid inhibitors, pharmaceutical compositions containing such compounds, and methods for using these compounds in the treatment of HIV infection. The invention also relates to methods for making the compounds hereinafter described.

### BACKGROUND OF THE INVENTION

Acquired immunodeficiency syndrome (AIDS) is the result of infection by HIV. HIV continues to be a major global public health issue. In 2015, an estimated 36.7 million people were living with HIV (including 1.8 million children)—a global HIV prevalence of 0.8%. The vast majority of this number live in low- and middle-income countries. In the same year, 1.1 million people died of AIDS-related illnesses.

Current therapy for HIV-infected individuals consists of a combination of approved anti-retroviral agents. Close to four dozen drugs are currently approved for HIV infection, either as single agents, fixed dose combinations or single tablet regimens; the latter two containing 2-4 approved agents. These agents belong to a number of different classes, targeting either a viral enzyme or the function of a viral protein during the virus replication cycle. Thus, agents are classified as either nucleotide reverse transcriptase inhibitors (NRTIs), non-nucleotide reverse transcriptase inhibitors (NNRTIs), protease inhibitors (PIs), integrase strand transfer inhibitors (INSTIs), or entry inhibitors (one, maraviroc, targets the host CCR5 protein, while the other, enfuvirtide, is a peptide that targets the gp41 region of the viral gp160 protein). In addition, a pharmacokinetic enhancer (cobicistat or ritonavir) can be used in combinations with antiretroviral agents (ARVs) that require boosting.

Despite the armamentarium of agents and drug combinations, there remains a medical need for new anti-retroviral agents. High viral heterogeneity, drug-associated toxicity, tolerability problems, and poor adherence can all lead to treatment failure and may result in the selection of viruses with mutations that confer resistance to one or more antiretroviral agents or even multiple drugs from an entire class (Beyrer, C., Pozniak A. HIV drug resistance—an emerging threat to epidemic control. N. Engl. J. Med. 2017, 377, 1605-1607; Gupta, R. K., Gregson J., et al. HIV-1 drug resistance before initiation or re-initiation of first-line antiretroviral therapy in low-income and middle-income countries: a systematic review and meta-regression analysis. Lancet Infect. Dis. 2017, 18, 346-355; Zazzi, M., Hu, H., Prosperi, M. The global burden of HIV-1 drug resistance in the past 20 years. PeerJ. 2018, DOI 10.7717/peerj.4848). As a result, new drugs are needed that are easier to take, have high genetic barriers to the development of resistance and have improved safety over current agents. In this panoply of choices, novel mechanisms of action (MOAs) that can be used as part of the preferred antiretroviral therapy (ART) can still have a major role to play since they should be effective against viruses resistant to current agents.

Certain potentially therapeutic compounds have now been described in the art and set forth in Blair, Wade S. et. al. Antimicrobial Agents and Chemotherapy (2009), 53(12), 5080-5087, Blair, Wade S. et al. PLoS Pathogens (2010), 6(12), e1001220, Thenin-Houssier, Suzie; Valente, Susana T. Current HIV Research, 2016, 14, 270-282, and PCT Patent applications with the following numbers: WO 2012065062, WO 2013006738, WO 2013006792, WO 2014110296, WO 2014110297, WO 2014110298, WO 2014134566, WO 2015130964, WO2015130966, WO 2016033243, WO2018035359, WO2018203235, WO 2019161017, and WO 2019161280.

What is now needed in the art are additional compounds which are novel and useful in the treatment of HIV. Additionally, these compounds should provide advantages for pharmaceutical uses, for example, with regard to one or more of their mechanisms of action, binding, inhibition efficacy, target selectivity, solubility, safety profiles, bioavailability or reduced frequency of dosing. Also needed are new formulations and methods of treatment which utilize these compounds.

### SUMMARY OF THE INVENTION

In one aspect, the present invention discloses a compound of Formula I, or a pharmaceutically acceptable salt thereof:

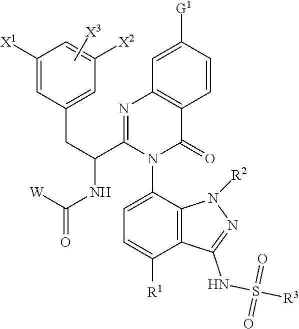

wherein:

X¹ and X² are independently selected from H, F, Cl or —CH₃ and X³ is H, F, Cl, —CH₃, —OCH₃, —OCHF₂, or —OCF₃ with the proviso that within the group X¹, X², and X³ the substituent Cl is not used more than twice and the substituent —CH₃ is not used more than twice;

R¹ is H, Cl, or CH₃;

R² is H, C₁-C₃alkyl optionally substituted with 1-3 fluorines, or C₃-C₆ cycloalkyl optionally substituted with 1-2 fluorines;

R³ is C₁-C₃alkyl or C₃-C₄ cycloalkyl;

G¹ is phenyl substituted once with —CO₂H or —CH₂O(C₁-C₃alkyl) wherein C₁-C₃alkyl is optionally substituted with 1-3 fluorines, or G¹ is fluorophenyl or difluorophenyl substituted once either at the ortho or meta position with C₁-C₂alkyl wherein C₁-C₂alkyl is substituted with 1-3 fluorines, or G¹ is phenyl, pyridine, pyrazine or pyrimidine substituted once with —SF₅, or G¹ is one of the following:

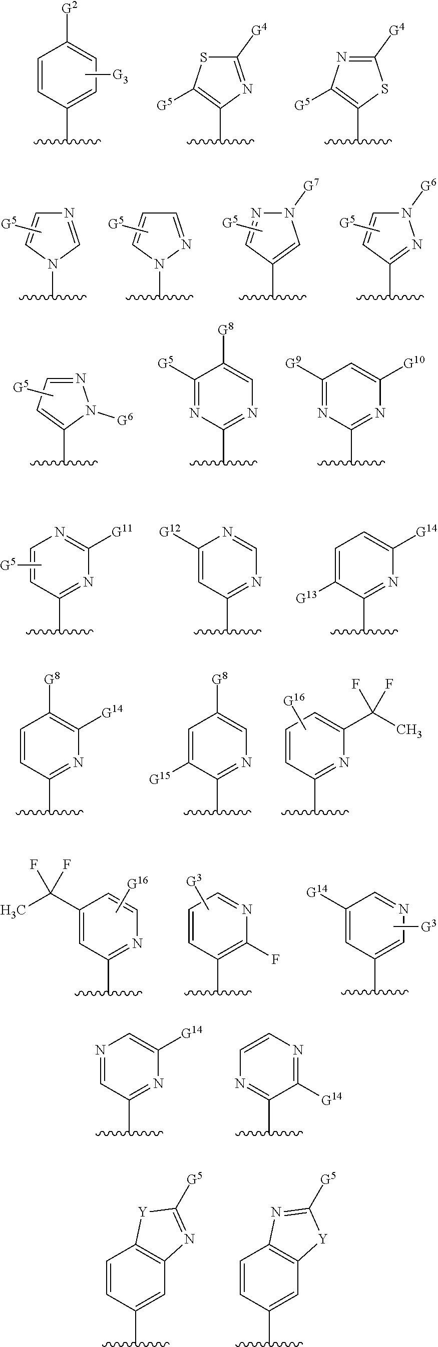

G² is —SO₂(C₁-C₃alkyl);

G³ is H, Cl, or F;

G⁴ is C₁-C₃alkyl substituted with 1-3 fluorines, or G⁴ is —O(C₁-C₃alkyl), —S(O₂)CH₃, or —C(CH₃)₂OH;

G⁵ is H, or methyl optionally substituted with 1-3 fluorines;

G⁶ is cyclopropyl, —CH₂cyclopropyl, C₁-C₃alkyl substituted with 1-5 fluorines, C₄alkyl optionally substituted with 1-5 fluorines, C₅alkyl, or —(C₂-C₃alkyl)O(C₁-C₂alkyl optionally substituted with 1-3 fluorines);

G⁷ is H, C₁-C₃alkyl or G⁶;

G⁸ is F, or Cl;

G₉ is H, —O(C₁-C₃alkyl) or C₁-C₃alkyl wherein C₁-C₃alkyl is optionally substituted with 1-3 fluorines;

G¹⁰ is —CN, —COCH₃, —SO₂(C₁-C₃alkyl) or Cl;

G¹¹ is —O(C₁-C₂alkyl optionally substituted with 1-3 fluorines), —SO₂(C₁-C₃alkyl), —CN, —CH₂F, —CHF₃, —CF₃, or —CF₂CH₃;

G¹² is methyl optionally substituted with 1-3 fluorines;

G¹³ is F, —CH₃, —CHF₂, —CF₃, —OCH₃, —SO₂CH₃;

G¹⁴ is C₁-C₂alkyl substituted with 1-3 fluorines;

G¹⁵ is H, F, Cl, —CH₃, —CH₂F, —CHF₂, —CF₃, OCH₃, —SO₂CH₃;

G¹⁶ is F, Cl, or methyl optionally substituted with 1-3 fluorines;

Y is O, S, or N;

W is selected from:

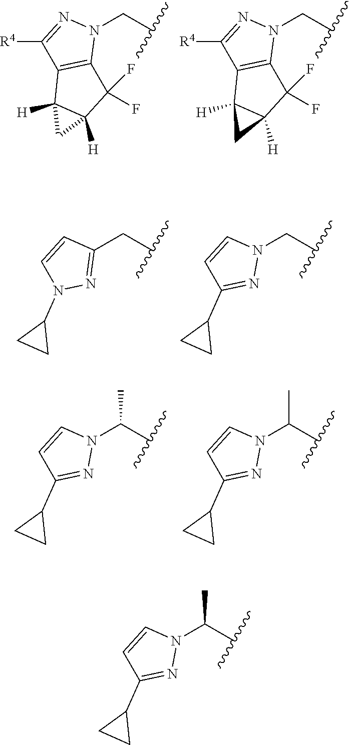

wherein R⁴ is methyl optionally substituted with 1-3 fluorines.

In another aspect, the present invention discloses a pharmaceutical composition comprising a compound of Formula I or a pharmaceutically acceptable salt thereof.

In another aspect, the present invention discloses a method of treating HIV infection in a human comprising administering a compound of Formula I or a pharmaceutically acceptable salt thereof.

In another aspect, the present invention discloses a compound of Formula I or pharmaceutically acceptable salt thereof for use in therapy.

In another aspect, the present invention discloses a compound of Formula I or pharmaceutically acceptable salt thereof for use in treating HIV infection in a human.

In another aspect, the present invention discloses the use of a compound of Formula I or pharmaceutically acceptable salt thereof in the manufacture of a medicament for the treatment of HIV infection in a human.

### DETAILED DESCRIPTION OF THE INVENTION

In one embodiment, the present invention discloses compounds of Formula I and pharmaceutically acceptable salts thereof wherein W is the following:

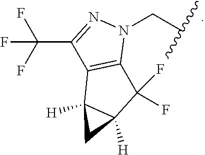 [SMILES: *Cn1nc(C(F)(F)F)c2c1C(F)(F)[C@H]1C[C@@H]21]

In one embodiment, the present invention discloses compounds of Formula I and pharmaceutically acceptable salts thereof wherein W is the following:

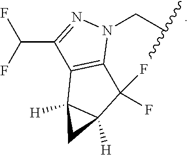 [SMILES: *Cn1nc(C(F)F)c2c1C(F)(F)[C@H]1C[C@@H]21]

In one embodiment, the present invention discloses compounds of Formula I and pharmaceutically acceptable salts thereof wherein W is the following:

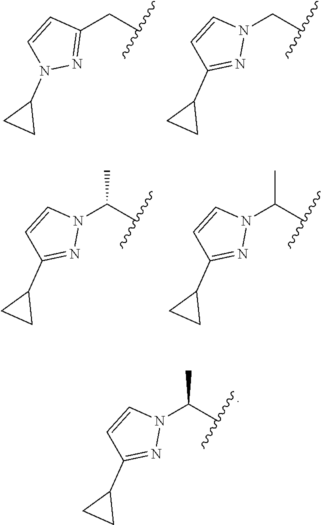 [SMILES: *Cc1ccn(C2CC2)n1 ; *Cn1ccc(C2CC2)n1 ; *[C@@H](C)n1ccc(C2CC2)n1 ; *C(C)n1ccc(C2CC2)n1 ; *[C@H](C)n1ccc(C2CC2)n1]

In one embodiment, the present invention discloses compounds of Formula I and pharmaceutically acceptable salts thereof wherein R¹ is Cl; R² is methyl, 2,2-difluoroethyl, or 2,2,2-trifluoroethyl; and R³ is methyl or cyclopropyl. In another embodiment, the present invention discloses compounds of Formula I and pharmaceutically acceptable salts thereof wherein R¹ is Cl; R² is methyl; and R³ is methyl. In another embodiment, the present invention discloses compound of Formula I and pharmaceutically acceptable salts thereof wherein R¹ is Cl; R² is 3-fluoropropyl; and R³ is methyl.

In one embodiment, the present invention discloses compounds of Formula I and pharmaceutically acceptable salts thereof wherein X³ is H. In another embodiment, the present invention discloses compounds of Formula I and pharmaceutically acceptable salts thereof wherein X¹ is F, X² is F, and X³ is H. In another embodiment, the present invention discloses compounds of Formula I and pharmaceutically acceptable salts thereof wherein if X³ is H then at least one of X¹ and X² is other than F. In another embodiment, the present invention discloses compounds of Formula I and pharmaceutically acceptable salts thereof wherein X¹ is H, X² is H and X³ is F.

In one embodiment, the present invention discloses compounds of Formula I and pharmaceutically acceptable salts thereof wherein G¹ is phenyl substituted once with —CO₂H or —CH₂O(C₁-C₃alkyl) wherein C₁-C₃alkyl is optionally substituted with 1-3 fluorines, or G¹ is fluorophenyl or difluorophenyl substituted once either at the ortho or meta position with C₁-C₂alkyl wherein C₁-C₂alkyl is substituted with 1-3 fluorines, or G¹ is phenyl, pyridine, pyrazine or pyrimidine substituted once with —SF₅, or G¹ is the following:

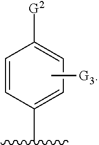

In one embodiment, the present invention discloses compounds of Formula I and pharmaceutically acceptable salts thereof wherein G¹ is one of the following:

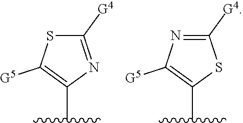

In one embodiment, the present invention discloses compounds of Formula I and pharmaceutically acceptable salts thereof wherein G¹ is one of the following:

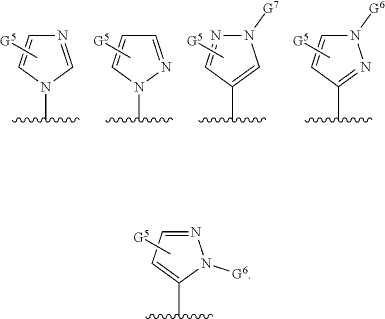

In one embodiment, the present invention discloses compounds of Formula I and pharmaceutically acceptable salts thereof wherein G¹ is one of the following

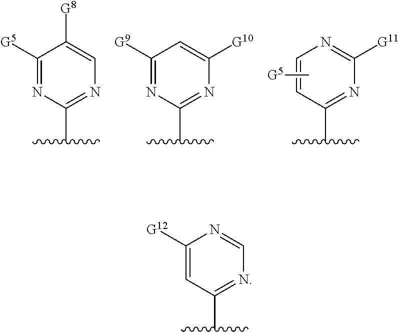

In one embodiment, the present invention discloses compounds of Formula I and pharmaceutically acceptable salts thereof wherein G¹ is one of the following:

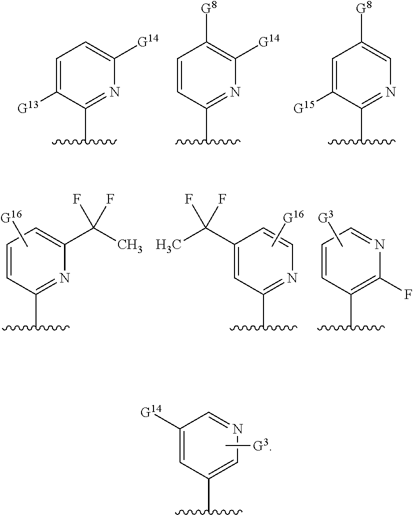

In one embodiment, the present invention discloses compounds of Formula I and pharmaceutically acceptable salts thereof wherein G¹ is one of the following:

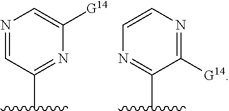

In one embodiment, the present invention discloses compounds of Formula I and pharmaceutically acceptable salts thereof wherein G¹ is one of the following:

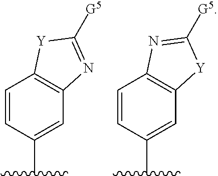

In one embodiment, the present invention discloses compounds of Formula I and pharmaceutically acceptable salts thereof wherein G¹ is one of the following:

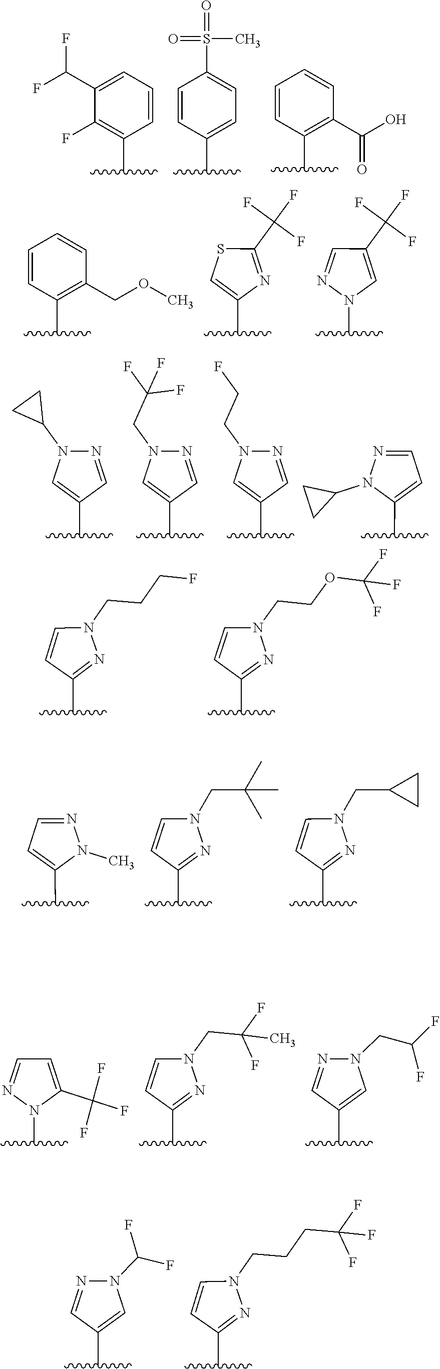

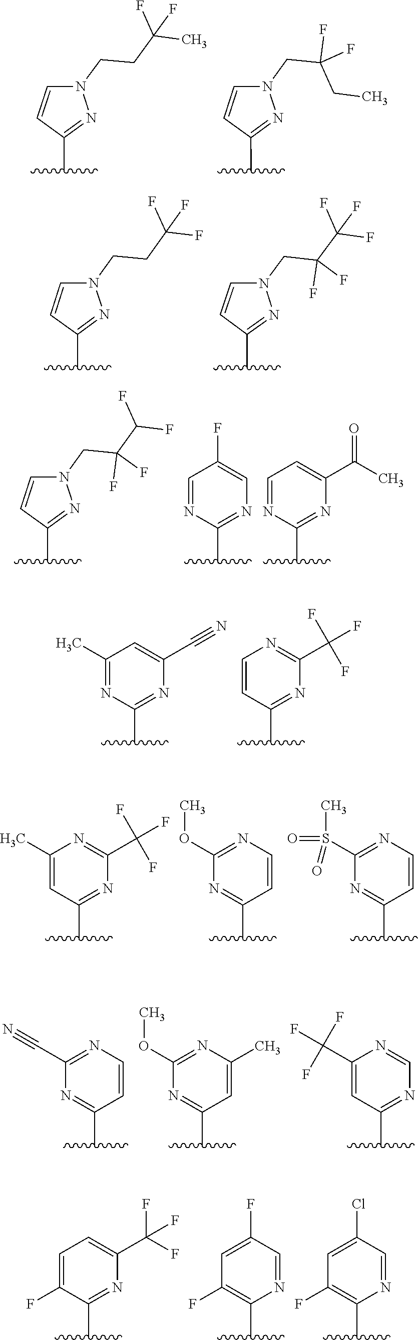

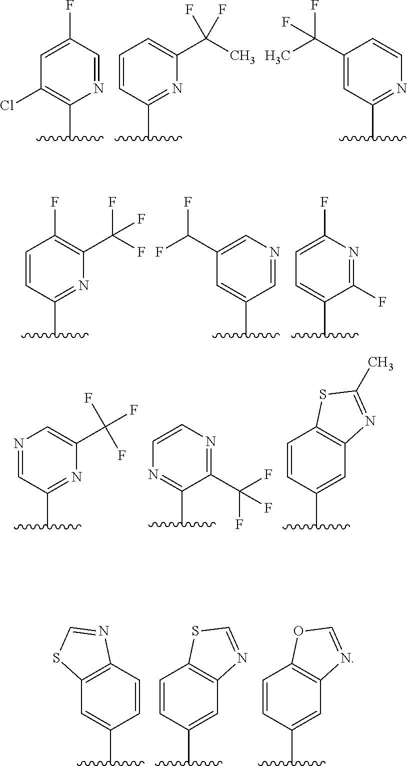

In one embodiment, the present invention discloses compounds of Formula I and pharmaceutically acceptable salts thereof wherein G¹ is one of the following:

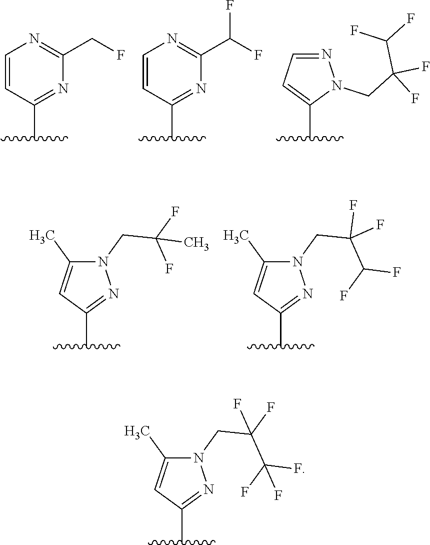

In one embodiment, the present invention discloses compounds of Formula I and pharmaceutically acceptable salts thereof wherein the stereochemistry is as depicted below:

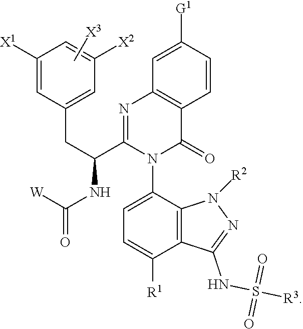

In one embodiment, the present invention discloses compounds of Formula I and pharmaceutically acceptable salts thereof wherein the stereochemistry is as depicted below:

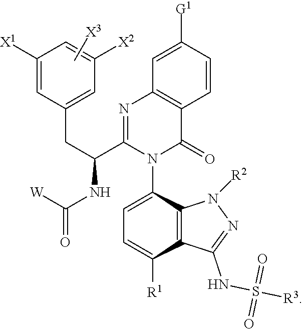

In one embodiment, the present invention discloses compounds or salts selected from the group consisting of:

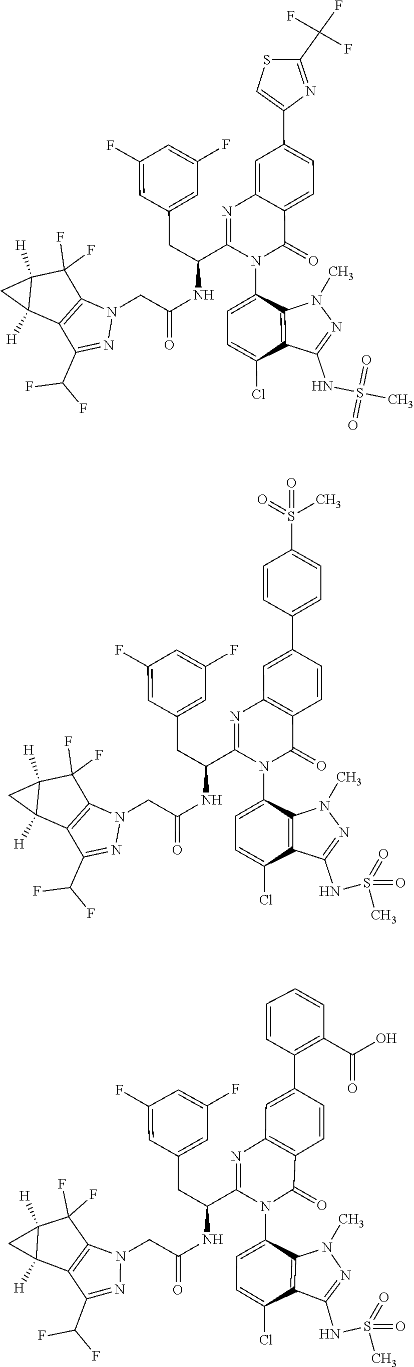

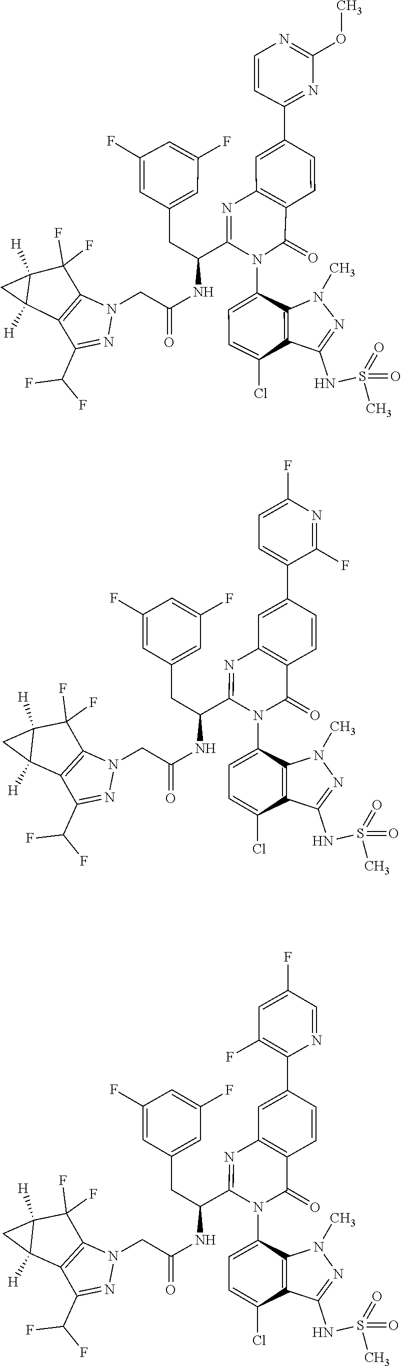

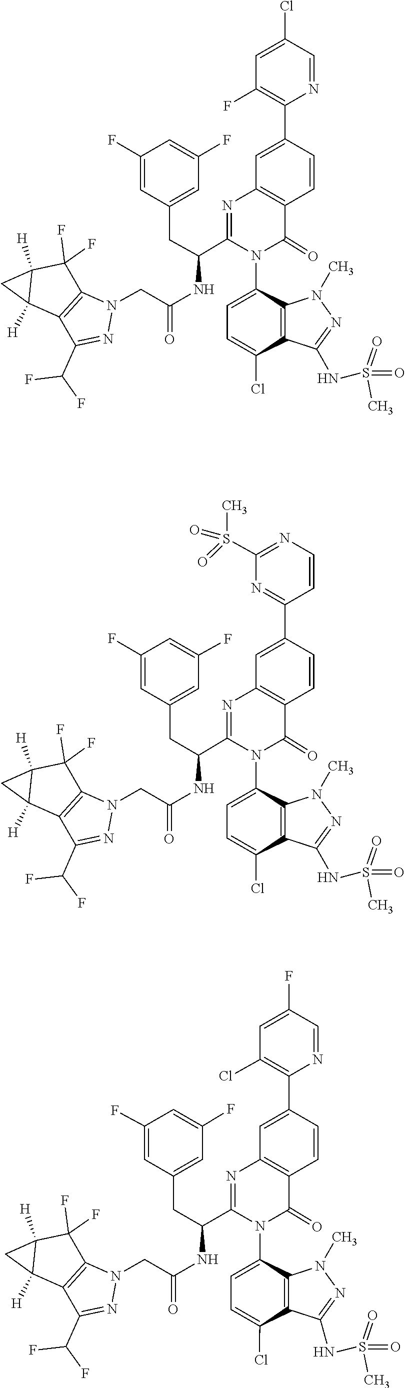

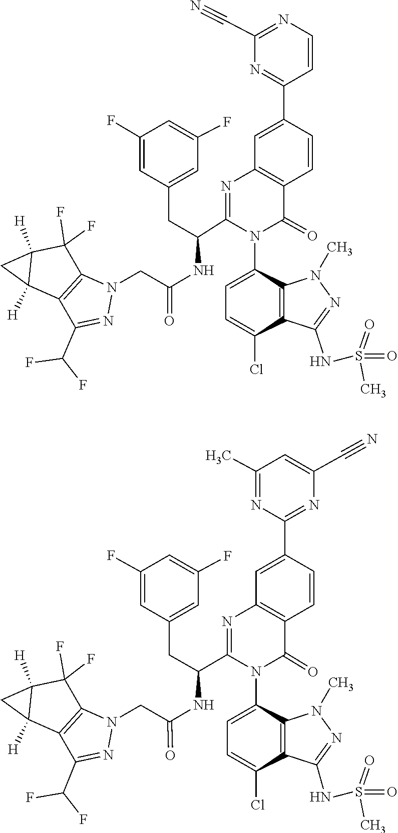

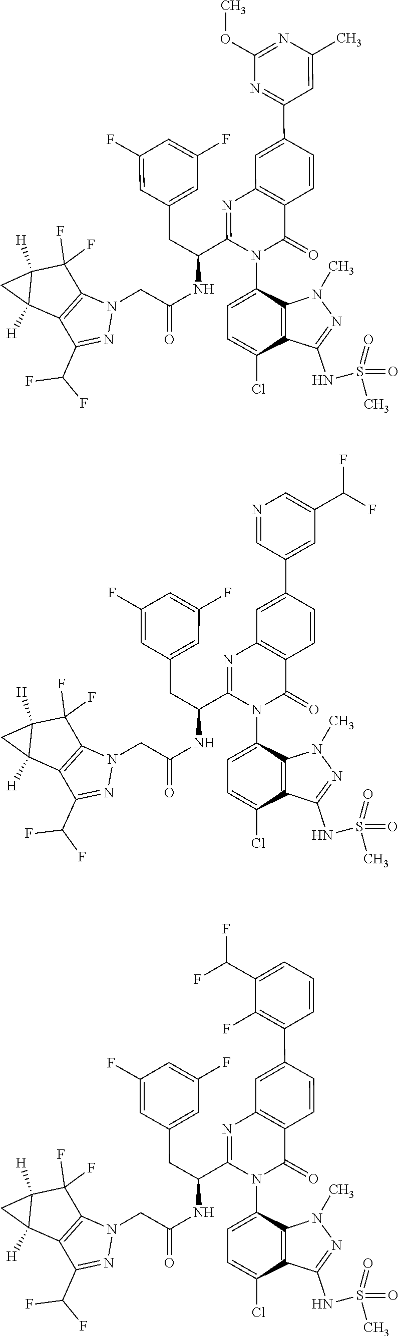

and pharmaceutically acceptable salts thereof.

In one embodiment, the present invention discloses compounds or salts selected from the group consisting of:

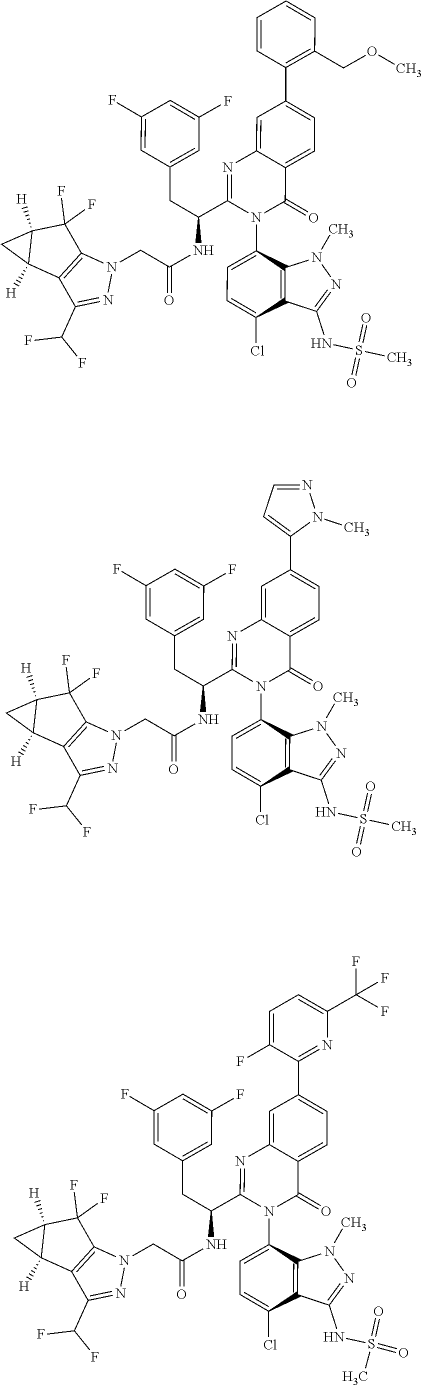

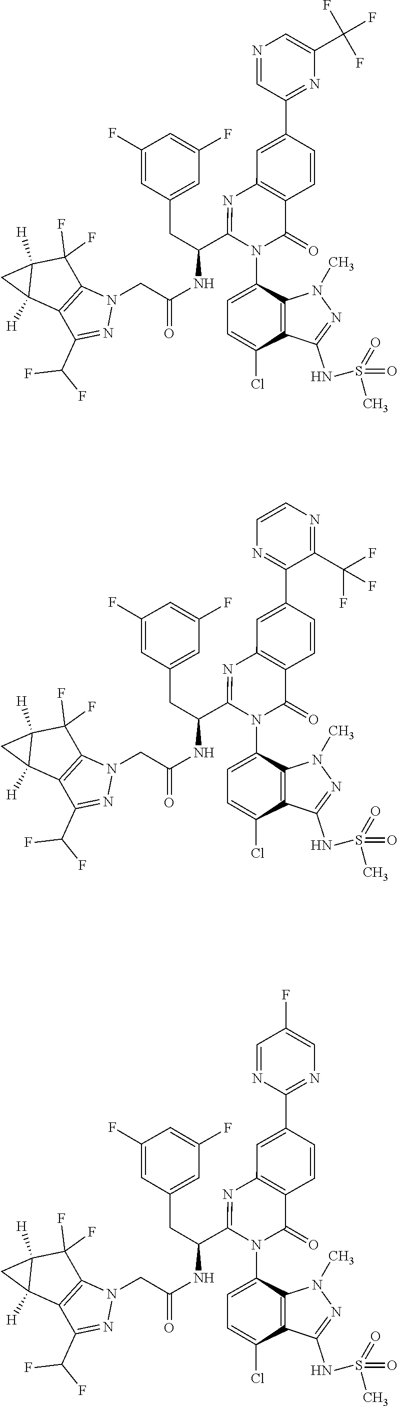

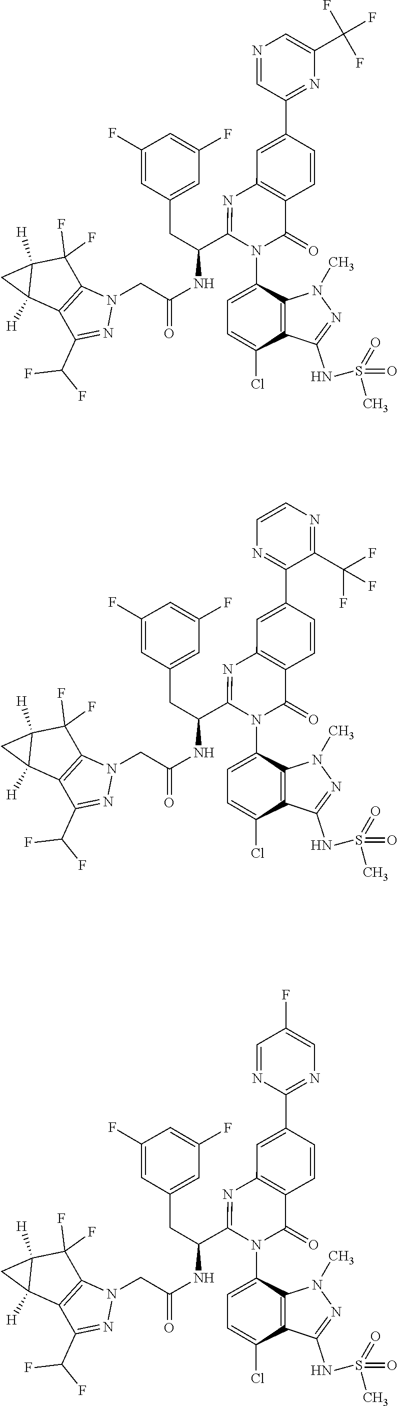

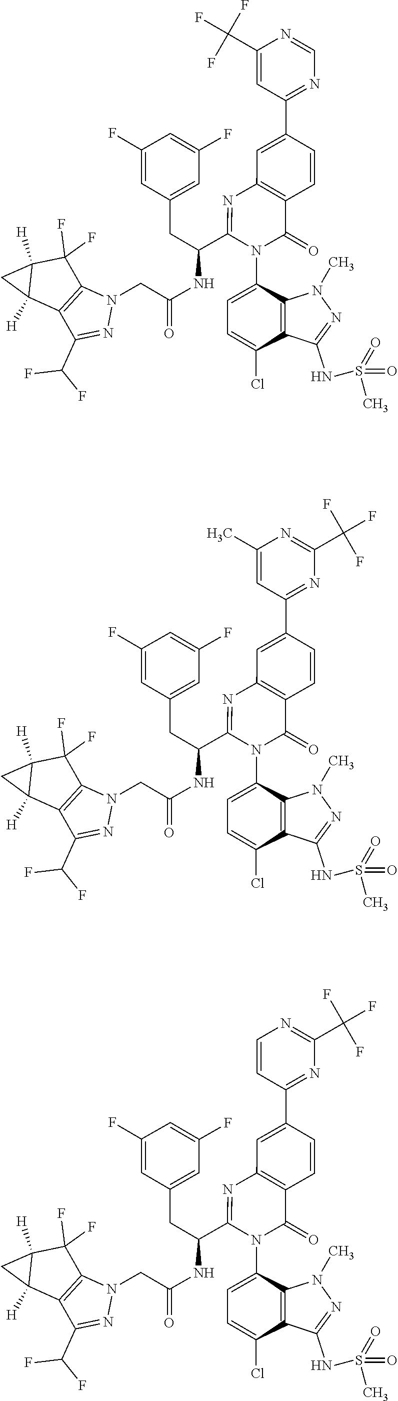

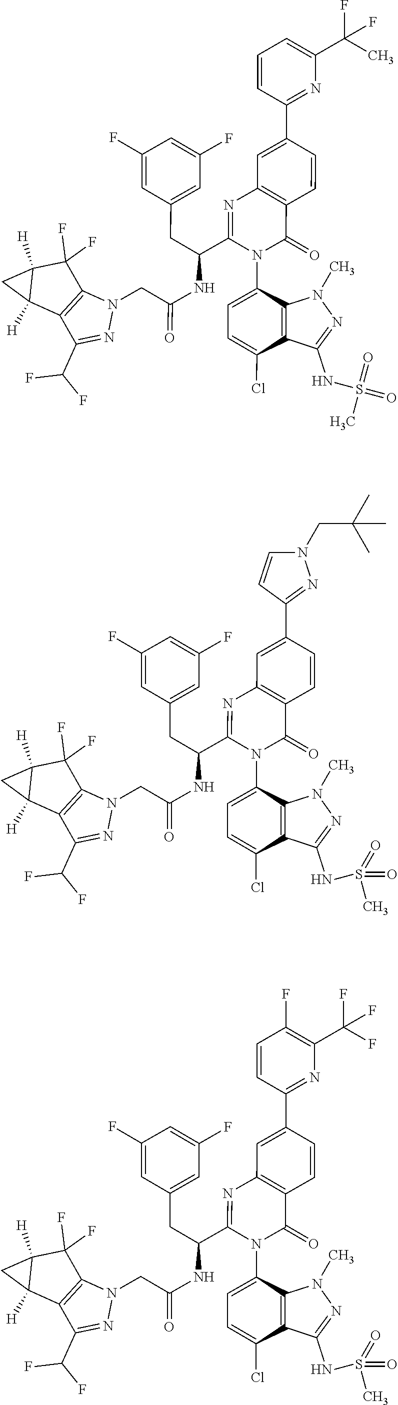

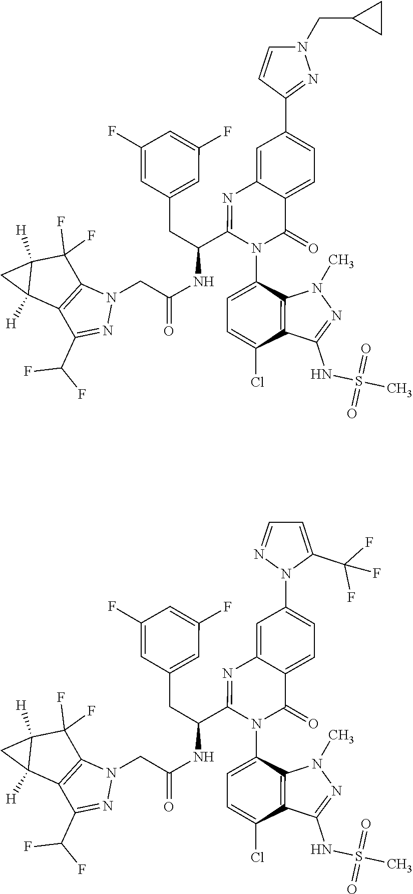

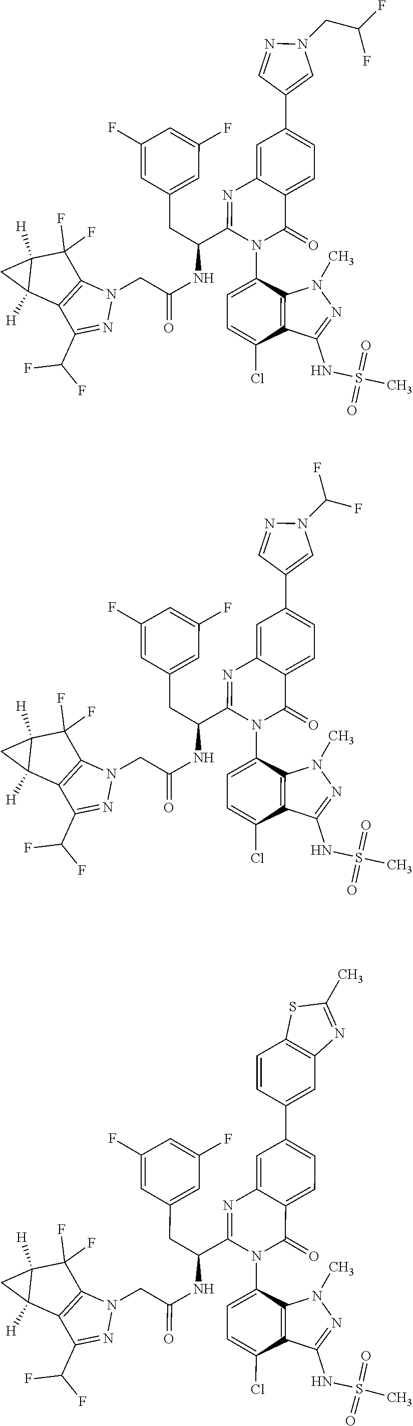

and pharmaceutically acceptable salts thereof.

In one embodiment, the present invention discloses compounds or salts selected from the group consisting of:

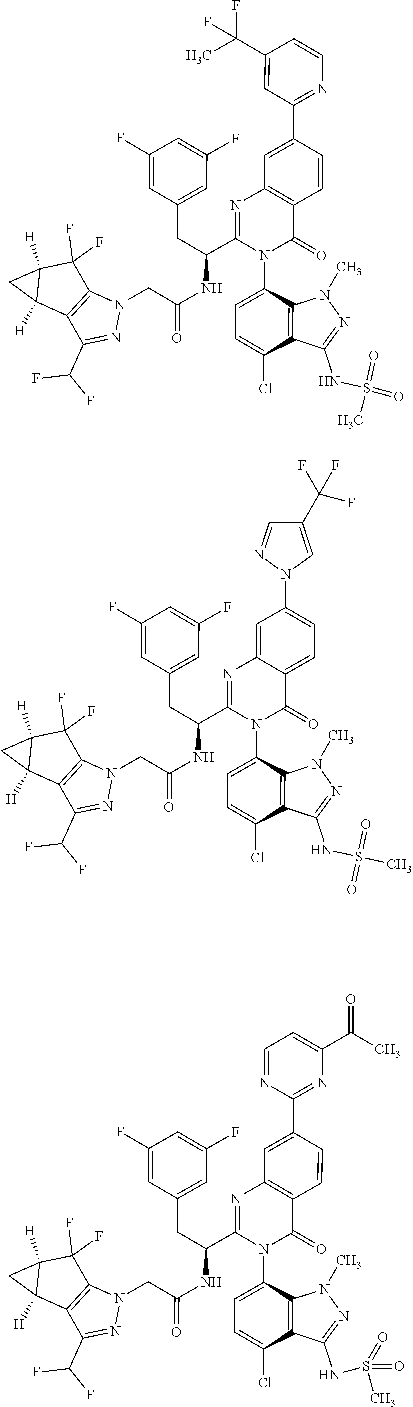

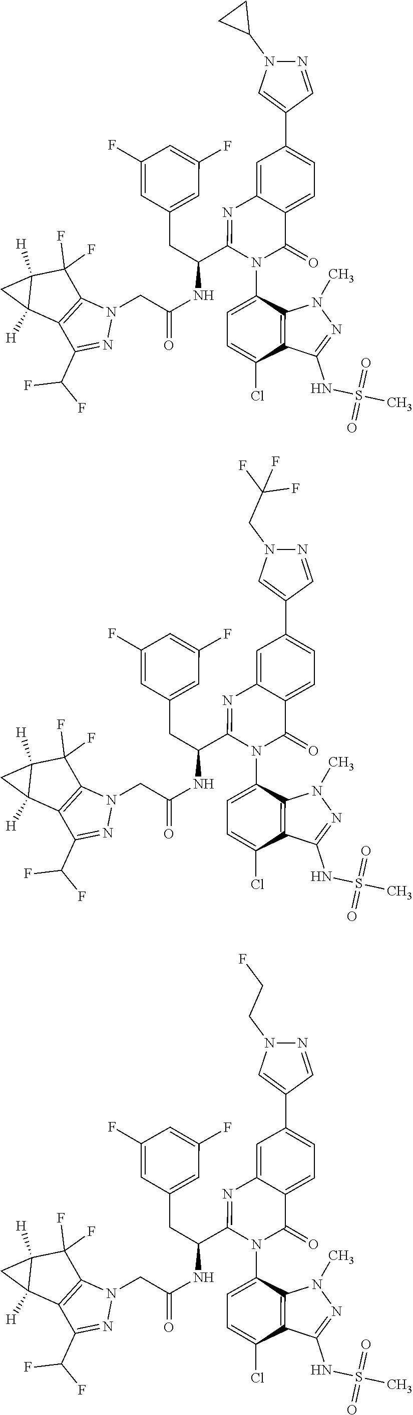

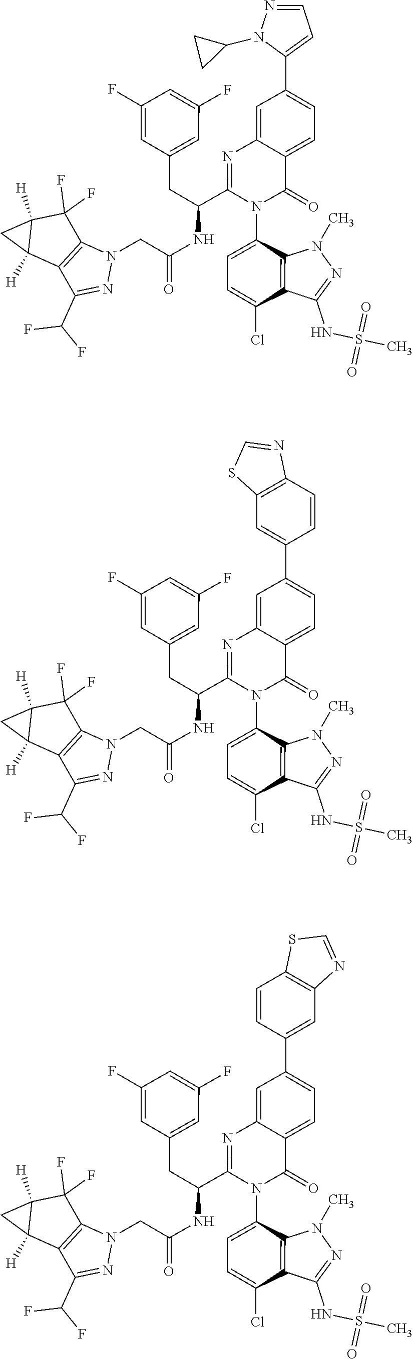

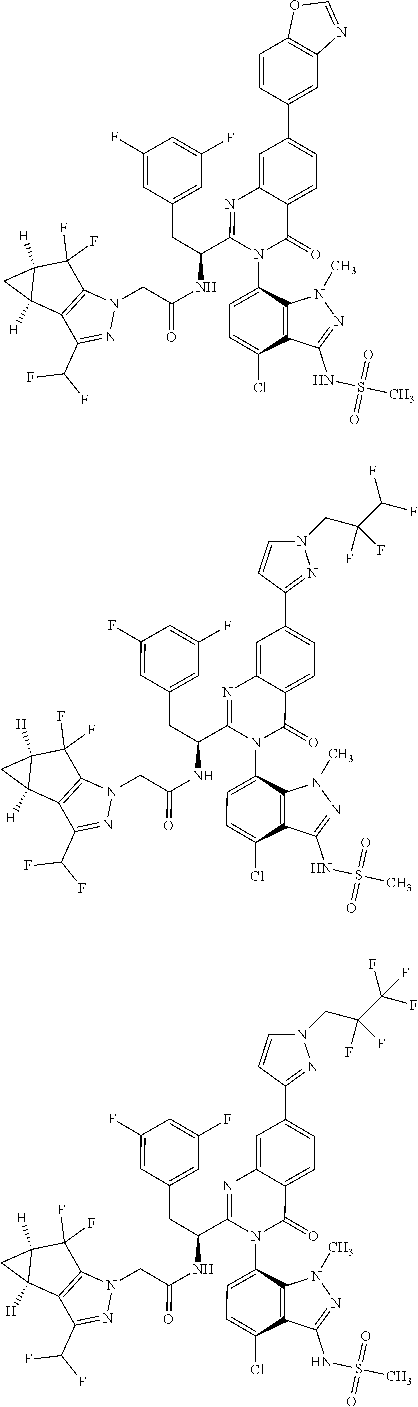

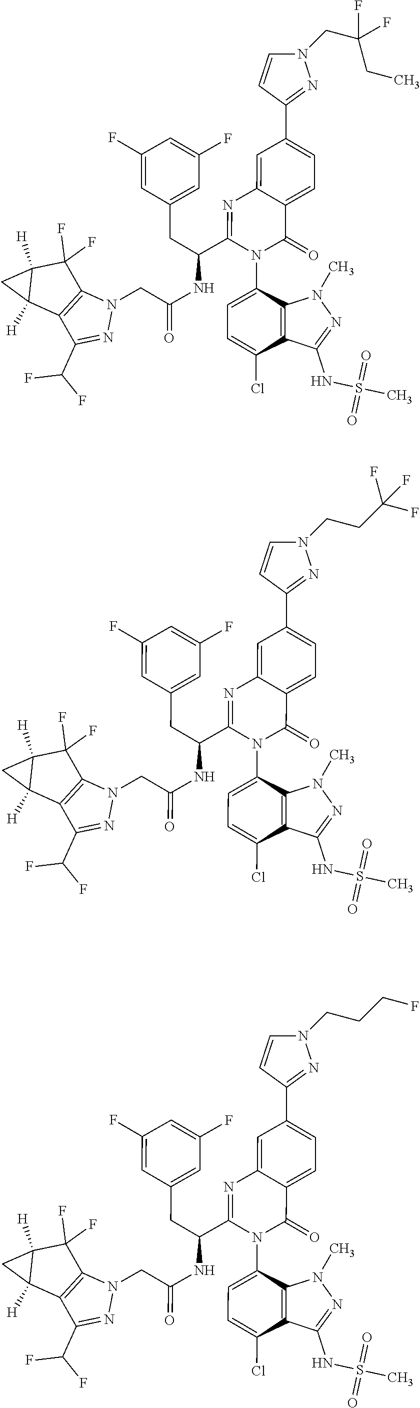

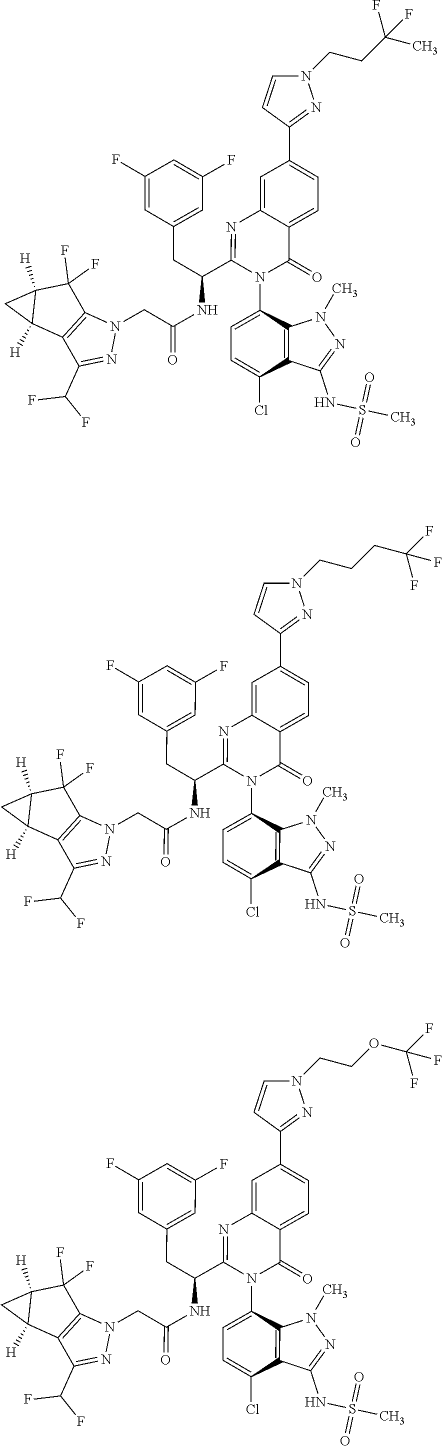

and pharmaceutically acceptable salts thereof.

In one embodiment, the present invention discloses compounds or salts selected from the group consisting of:

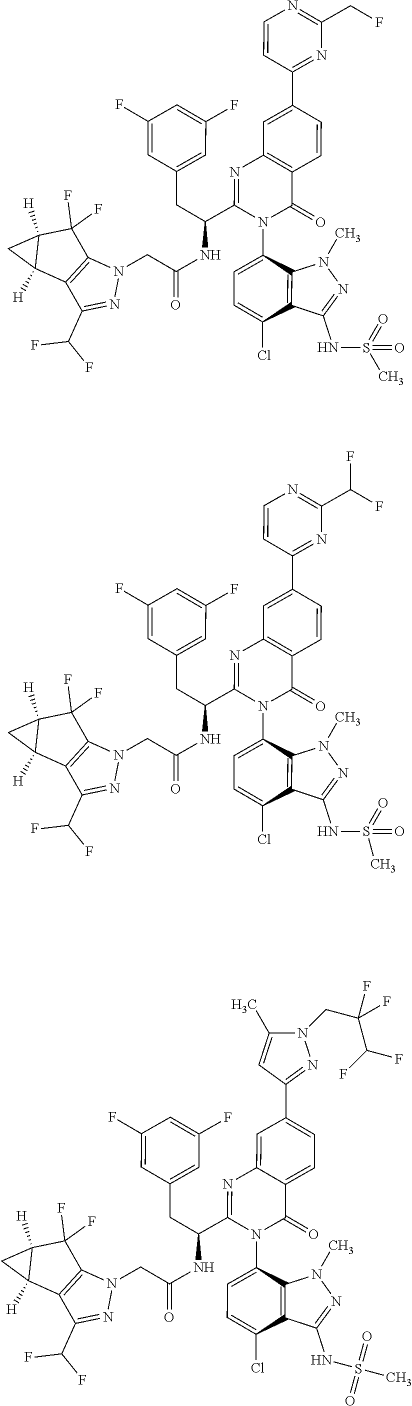

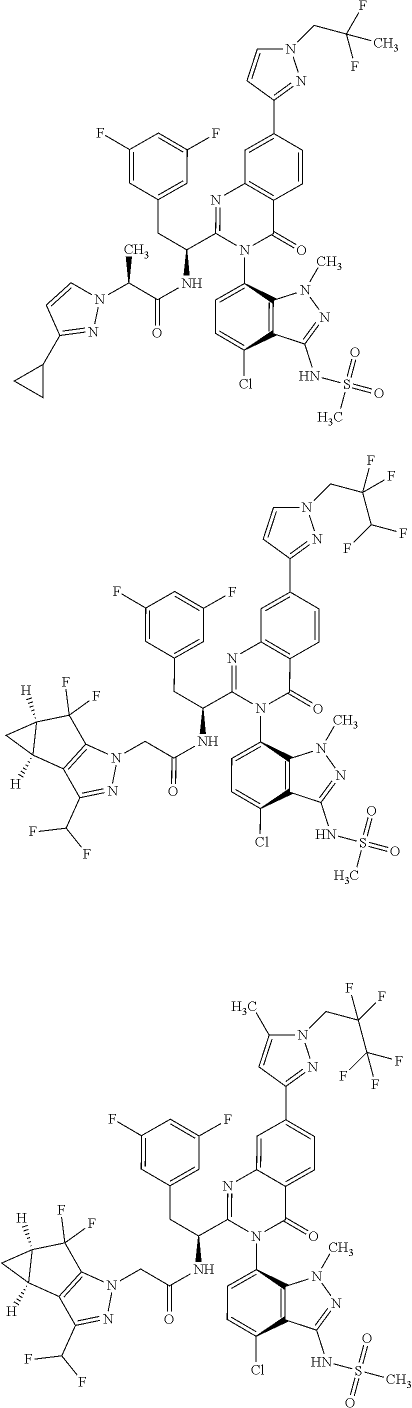

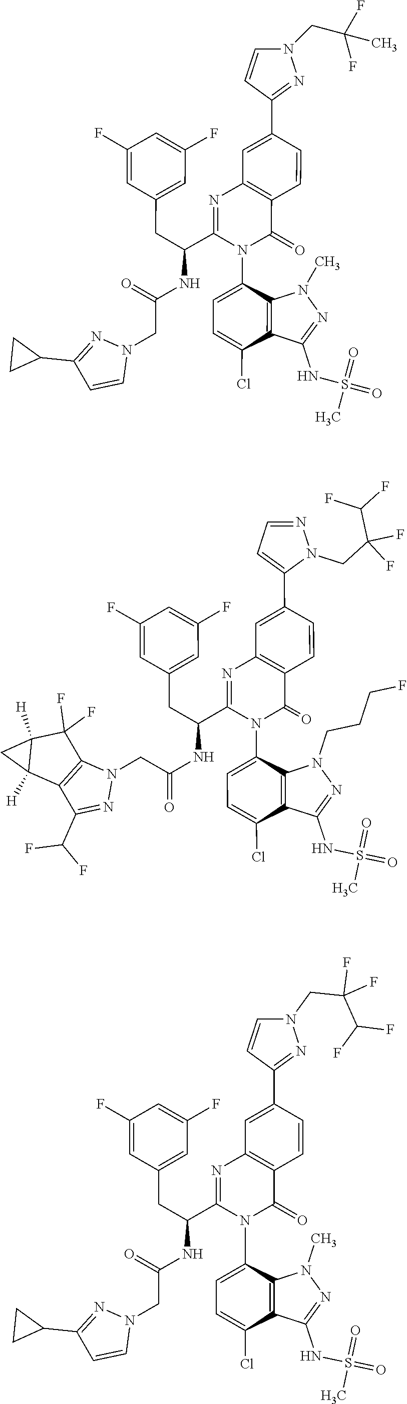

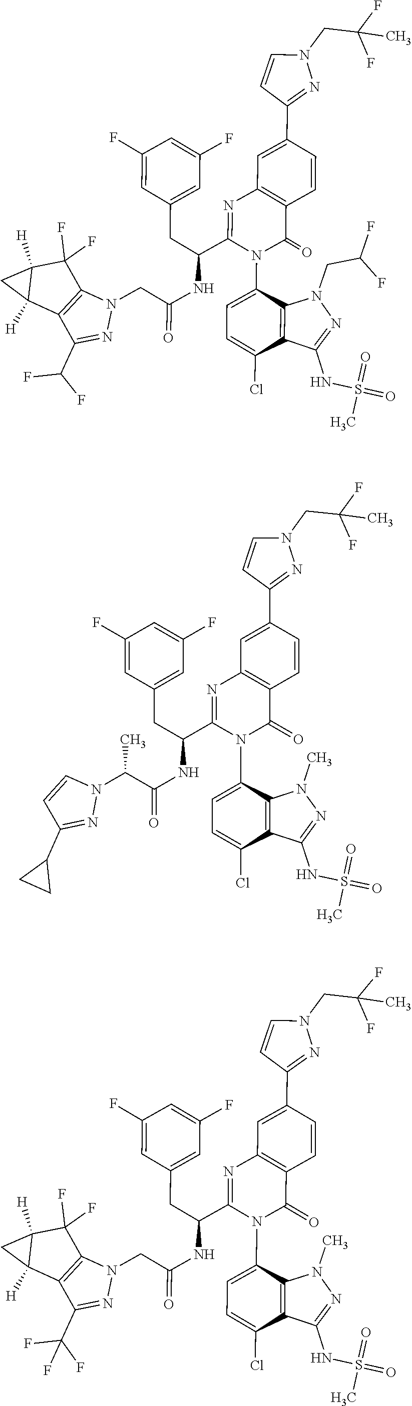

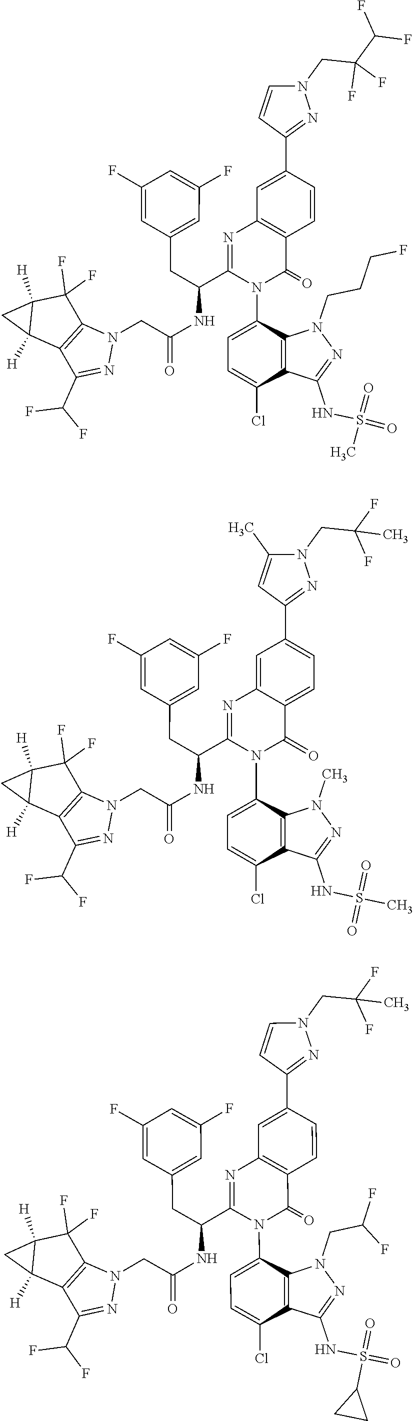

and pharmaceutically acceptable salts thereof.

In one embodiment, the present invention discloses the following compound:

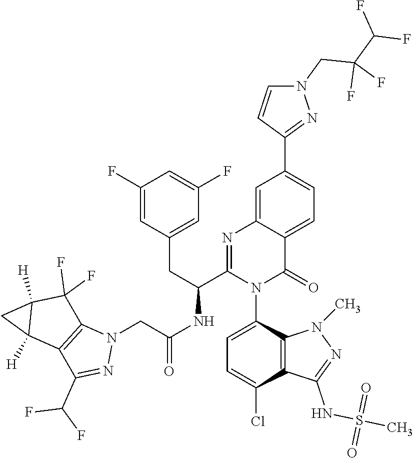

and pharmaceutically acceptable salts thereof.

In one embodiment, the present invention discloses the following compound:

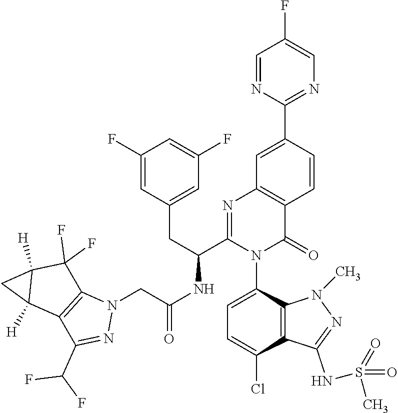

and pharmaceutically acceptable salts thereof.

The salts of the invention are pharmaceutically acceptable. Such salts may be acid addition salts or base addition salts. For a review of suitable pharmaceutically acceptable salts see, for example, Berge et al, J. Pharm, Sci., 66, 1-19, 1977.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (NV-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

In one embodiment, the compositions of this invention further comprise a pharmaceutically acceptable excipient. In the method of this invention, preferred routes of administration are oral and by injection to deliver subcutaneously or intramuscularly. Therefore, preferred pharmaceutical compositions include compositions suitable for oral administration (for example tablets) and compositions suitable for subcutaneous or intramuscular injection.

In another aspect the present invention discloses methods of preventing HIV infection in a human or reducing the risk of infection, comprising administering a compound or salt of this invention. Pre-exposure prophylaxis (or PrEP) is when people at risk for HIV infection take daily medicine to lower their chances of getting HIV infection. PrEP has been shown to be effective in reducing the risk of infection. As used herein, “HIV” or “Human Immunodeficiency Virus” refers to HIV-1 and/or to HIV-2.

The compounds and salts of this invention are believed to have as their biological target the HIV capsid and thus their mechanism of action is to modify in one or more ways the function of the HIV capsid.

The compounds and salts of the present invention may be employed alone or in combination with other therapeutic agents. Combination therapies according to the present invention thus comprise the administration of at least one compound or salt of the invention, and the administration of at least one other agent which may be useful in the treatment of HIV infection. A compound or salt of the present invention, and the other agent may be formulated and administered together in a single pharmaceutical composition or may be formulated and administered separately. When formulated and administered separately, administration may occur simultaneously or sequentially in any order. Suitable other agents include, for example, abacavir, atazanavir, bictegravir, cabotegravir, darunavir, delavirdine, didanosine, dideoxyinosine, dolutegravir, doravirine, efavirenz, elvitegravir, emtricitabine, etavirine, fosamprenavir, fostemsavir, GSK3640254, the antibody N6LS, GSK3739937/VH3739937 and GSK4000422/VH4000422, indinavir, lamivudine, lopinavir, maraviroc, nelfinavir, nevirapine, raltegravir, rilpiverine, ritonavir, saquinavir, slatravir, stavudine, tipranavir, tenofovir, tenofovir alafenamide, tenofovir disoproxil fumarate, zalcitabine, zidovudine, and S-648414. Preferred agents include, for example, bictegravir, cabotegravir, dolutegravir, fostemsavir, islatravir, and lamivudine. Particularly preferred agents include, for example, bictegravir, cabotegravir, dolutegravir, fostemsavir, and lamivudine.

### EXAMPLES

### LCMS Method B:

Column=Acquity BEH C18, 2.1×30 mm, 1.7 μm particles; Solvent A=0.1% Formic acid in 100% Water; Solvent B=0.1% Formic Acid in 100% Acetonitrile; Flow Rate=0.8 mL/min.; Start % B=5, Final % B=95; Gradient Time=1.7 min, then a 0.2 min hold at 95% B. Detection=215 and 254 nm.

### LCMS Method C:

Column=Acquity UPLC BEH C18, 2.1×50 mm, 1.7 μm particles; Solvent A: 95:5 Water:MeCN w/ 0.1% TFA; Solvent B=5:95 Water:MeCN w/ 0.1% TFA; Flow rate=0.80 mL/min.; Start % B=0, End % B=100; Gradient Time=3.0 min., then a hold at 100% B for 1 minute. Detection=220 nm and 254 nm.

### LCMS Method E:

Column=Zorbax Eclipse Plus C18, 2.1×50 mm, 1.7 μm particles; Solvent A=0.1% Formic acid in 100% Water; Solvent B=0.1% Formic Acid in 100% Acetonitrile; Flow Rate=1 mL/min; Start % B=5, Final % B=95; Gradient Time=2.1 min, then a 0.3 min hold at 95% B. Detection=215 and 254 nm.

### LCMS Method F:

Column=Waters XTerra C18, 4.6×50 mm, 5 μm particles; Solvent A=0.1% NH₄OH in 100% Water; Solvent B=Acetonitrile; Flow Rate=2.5 mL/min.; Start % B=5, Final % B=95; Gradient Time=4 min, then a 1 min hold at 95% B. Detection=215 nm and 254 nm.

### LCMS Method H:

Column=Acquity CSH C18, 2.1×30 mm, 1.7 μm particles; Solvent A=0.1% Formic acid in 100% Water; Solvent B=0.1% Formic Acid in 100% Acetonitrile; Flow Rate=0.8 mL/min.; Start % B=5, Final % B=95; Gradient Time=1.7 min, then a 0.2 min hold at 95% B. Detection=215 and 254 nm.

### General Procedure K:

In a dry 1 dram vial equipped with a stir bar was combined N—((S)-1-((3P)-7-bromo-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (or other coupling partner as indicated) (1 equiv, typically 0.028-0.037 mmol), tribasic potassium phosphate (3 equiv), dichloro[9,9-dimethyl-4,5-bis(diphenylphosphino)xanthene]palladium(II) (0.05-0.1 equiv) and the indicated boronic acid or boronic ester (2-3 equiv). The vial was purged with argon and then was sealed with a septum cap. To the vial was added THF:water (4:1, 0.05M relative to trifluoromethanesulfonate). The mixture was stirred at either ambient temperature or 60° C. for 1-18 h (typically 18 h). Upon cooling to ambient temperature, the reaction was concentrated and the residue was subjected to HPLC purification to afford the indicated product.

### General Procedure L:

To a vial equipped with a stir bar and placed under argon atmosphere was added Pd(OAc)₂ (0.1 equiv), dicyclohexyl(2′,6′-dimethoxy-[1,1′-biphenyl]-2-yl)phosphane (0.2 equiv), tribasic potassium phosphate (3 equiv), N—((S)-1-((3P)-3-(4-chloro-1-methyl-3-(methylsulfonamido)-1H-indazol-7-yl)-4-oxo-7-(4,4,5,5-tetramethyl-1,3,2-dioxaborolan-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (or other boronic ester as indicated) (1 equiv, typically 0.040-0.043 mmol), and the appropriate aryl/heteroaryl halide (3 equiv). The vial was sealed with a septum cap. To the vial was added THF:water (4:1) to afford a reaction volume 0.05M in boronic ester. The reaction mixture was degassed with argon (the vial is briefly evacuated under vacuum and refilled with Ar, repeated three times; minor effervescence is observed during vacuum), then the reaction mixture was stirred at either ambient temperature or 45° C. for 16 to 48 h (typically 18 h). Upon cooling to ambient temperature, the reaction mixture was concentrated in vacuo and the resulting residue was subjected to HPLC purification to afford the indicated product.

### General Procedure M:

To a solution of N—((S)-1-((3P)-3-(4-chloro-3-(N-(4-methoxybenzyl)methylsulfonamido)-1-methyl-1H-indazol-7-yl)-4-oxo-7-(1H-pyrazol-3-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (or other pyrazole as indicated) (25 mg, 0.025 mmol) and the indicated triflate (0.076 mmol) in acetonitrile (1 mL) was added cesium carbonate (12.31 mg, 0.038 mmol). The resulting mixture was heated at 50° C. for 1 h, then the mixture was then cooled to room temperature, filtered and concentrated under reduced pressure. The resulting residue was taken up in DCM (0.5 mL) and to the mixture was added TFA (1 mL) and then triflic acid (0.05 mL). The mixture was stirred at rt for 1 h and then was concentrated in vacuo. The resulting residue was then taken up in DMF (2 mL), filtered, and the filtrate was subjected to HPLC purification to afford the indicated product.

### General HPLC Conditions:

HPLC purification was performed using one of the conditions indicated below, optionally followed by a second HPLC purification using a different condition indicated below. Based on analytical HPLC data obtained on the crude reaction mixture, the purification condition was optimized for each target compound by modifying the initial Solvent A:Solvent B ratio, the gradient time, the final Solvent A:Solvent B ratio, and the hold time at the final Solvent A:Solvent B concentration.
HPLC Condition A: Column: Zorbax Eclipse Plus C18, 21.2×100 mm, 5 μm particles; Solvent A=0.1% Formic Acid in 100% Water. Solvent B=Acetonitrile. Flow Rate=40 mL/min. Wavelength=215 and 254 nm. ESI+ Range: 150 to 1500 dalton.
HPLC Condition B: Column: Sunfire Prep C18 OBD, 30×100 mm, 5 μm particles; Solvent A: water:MeCN 95:5 w/ 0.1% TFA, Solvent B: MeCN:water 95:5 w/ 0.1% TFA. Flow Rate=42 mL/min. Wavelength=220 and 254 nm.
HPLC Condition C: Column: Waters Xterra C18, 19×100 mm, 10 μm particles; Solvent A=0.1% NH4OH in 100% Water. Solvent B=Acetonitrile. Flow Rate=40 mL/min. Wavelength=215 and 254 nm. ESI+ Range: 150 to 1500 dalton.
HPLC Condition D: Column: Waters XSelect CSH C18, 19×100 mm, 5 μm particles; Solvent A=0.1% Formic Acid in 100% Water. Solvent B=Acetonitrile. Flow Rate=40 mL/min. Wavelength=215 and 254 nm. ESI+ Range: 150 to 1500 dalton.

### Preparation of bicyco[3.1.0]hexan-3-ol

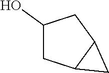

To a stirred solution of cyclopent-3-enol (130 g, 1545 mmol) in DCM (1200 mL) under N2 atmosphere at 0-5° C. was added dropwise a solution of diethyl zinc in hexane (1.0 M, 3091 mL, 3091 mmol) over a period of 3 h. To the solution at 0° C. was added dropwise a solution of diiodomethane (249 mL, 3091 mmol) in DCM (300 mL) over a period of 1 h. The reaction mixture was allowed to warm to 27° C. upon which formation of a white precipitation was observed. The mixture stirred for 16 h. Progress of the reaction was monitored by TLC (SiO₂, 20% EtOAc/pet, Rf=0.3, UV-inactive, PMA-active). The reaction mixture was quenched via the careful addition of aq. saturated NH₄Cl solution (1.5 L). The mixture was filtered through pad of Celite. The aqueous layer was extracted with DCM (2×1 L). The combined organic layers were dried over anhydrous Na₂SO₄, filtered and then concentrated under reduced pressure to afford crude bicyclo[3.1.0]hexan-3-ol as red liquid, 180 g. ¹H NMR (400 MHz, CDCl₃) δ=4.41-4.35 (m, 1H), 2.18-2.05 (m, 2H), 1.73 (d, J=13.9 Hz, 2H), 1.35-1.25 (m, 2H), 1.21-1.14 (m, 1H), 0.57-0.43 (m, 2H). GCMS: m/z=98.1).

### Preparation of bicyclo[3.1.0]hexan-3-one

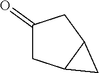

To a stirred solution of bicyclo[3.1.0]hexan-3-ol (210 g, 2054 mmol) in DCM (5000 mL) under N2 atmosphere at 0° C. was added portion-wise Dess-Martin periodinane (954 g, 225 mmol). The mixture was allowed to warm to 27° C. and was then stirred for 16 h. Progress of the reaction was monitored by TLC (SiO₂, 20% Acetone/Hex, Rf=0.3, UV in-active, PMA-active). The reaction mixture was filtered through pad of Celite and the filtrate was washed with aq. NaOH (1N, 8×1 L). The combined aqueous phases were extracted with DCM (5×1 L). The combined organic layers were dried over anhydrous Na₂SO₄, filtered, and then concentrated under reduced pressure (bath temperature: 20° C.) to afford crude bicyclo[3.1.0]hexan-3-one as brown liquid. The liquid was further purified by downward distillation at 70° C. to afford bicyclo[3.1.0]hexan-3-one as a pale-yellow viscous liquid, 125 g (62%). ¹H NMR (400 MHz, CDCl₃) δ=2.61-2.54 (m, 2H), 2.17-2.12 (m, 2H), 1.54-1.46 (m, 2H), 0.92-0.86 (m, 1H), −0.01-−0.08 (m, 1H); GCMS: M/Z=96.1.

### Preparation of 2-(2,2-difluoroacetyl)bicyclo[3.1.0]hexan-3-one

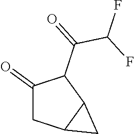 [SMILES: O=C1CC2CC2C1C(=O)C(F)F]

To a stirred solution of bicyclo[3.1.0]hexan-3-one (125 g, 1274 mmol) in THF (1500 mL) under N2 atmosphere at −78° C. was added LDA (2.0 M in THF, 0.701 L, 1402 mmol). The solution was stirred for 1 h at −78° C. To the solution was added slowly over 30 minutes a solution of ethyldifluoroacetate (174 g, 1402 mmol) in THF (300 mL) maintaining a temperature of −78° C. The reaction mixture was allowed to warm to 27° C. and was then stirred for 1 h. Progress of the reaction was monitored by TLC (SiO₂, 20% Acetone/Hexane, Rf=0.3, UV-active). The reaction mixture was quenched via the addition of aq. HCl (1N, 2000 mL). The mixture was stirred for 30 min. and then was extracted with EtOAc (3×1000 mL). The combined organic layers were washed with brine (1000 mL), dried over anhydrous Na₂SO₄ and filtered. The filtrate was concentrated under reduced pressure to afford 2-(2,2-difluoroacetyl)bicyclo[3.1.0]hexan-3-one as a pale-yellow viscous liquid, 180 g (71%). ¹H NMR (400 MHz, CDCl₃) δ=6.18 (t, J=54.8 Hz, 1H), 2.70-2.62 (m, 1H), 2.35 (d, J=19.4 Hz, 1H), 2.14 (br s, 1H), 1.26-1.21 (m, 1H), 1.04-1.03 (m, 1H), 0.22-0.21 (m, 1H), LCMS: M/Z=173.17).

### Preparation of ethyl 2-(3-(difluoromethyl)-3b,4,4a,5-tetrahydro-1H-cyclopropa[3,4]cyclopenta[1,2-c]pyrazol-1-yl)acetate

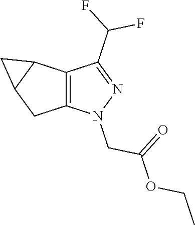 [SMILES: CCOC(=O)Cn1nc(C(F)F)c2c1CC1CC21]

To a stirred solution of 2-(2,2-difluoroacetyl)bicyclo[3.1.0]hexan-3-one (180 g, 910 mmol) in ethanol (2 L) under N2 atmosphere at 27° C. was added ethyl 2-hydrazinylacetate hydrochloride (422 g, 2729 mmol) followed by sulfuric acid (20 mL, 375 mmol). The mixture was stirred for 30 min. and then was heated to 100° C. and stirred for 16 h. Progress of the reaction was monitored by TLC (SiO₂, 20% Acetone/Hexane, Rf=0.3, UV-active). The reaction mixture was concentrated under reduced pressure. The residue was dissolved in EtOAc (2000 mL) and was washed with water (2×1 L), brine (1.0 L), dried over anhydrous Na₂SO₄, filtered, and then was concentrated under reduced pressure. The resulting residue was subjected to silica gel column chromatography (pet.:acetone 100:0-498:2) to afford ethyl 2-(3-(difluoromethyl)-3b,4,4a,5-tetrahydro-1H-cyclopropa[3,4]cyclopenta[1,2-c]pyrazol-1-yl)acetate as an off-white solid, 110 g (46%). ¹H NMR (400 MHz, DMSO-d₆) δ=6.86 (t, J=54.8 Hz, 1H), 4.93 (s, 2H), 4.14 (q, J=7.2 Hz, 2H), 2.88-2.79 (m, 1H), 2.76-2.68 (m, 1H), 2.14-2.04 (m, 2H), 1.19 (t, J=7.2 Hz, 3H), 1.10-1.03 (m, 1H), 0.14 (q, J=4.3 Hz, 1H).

### Preparation of ethyl 2-(3-(difluoromethyl)-5-oxo-3b,4,4a,5-tetrahydro-1H-cyclopropa[3,4]cyclopenta[1,2-c]pyrazol-1-yl)acetate

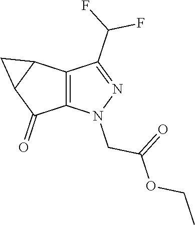 [SMILES: CCOC(=O)Cn1nc(C(F)F)c2c1C(=O)C1CC21]

To a stirred solution of ethyl 2-(3-(difluoromethyl)-3b,4,4a,5-tetrahydro-1H-cyclopropa[3,4]cyclopenta[1,2-c]pyrazol-1-yl)acetate (110 g, 422 mmol) and Celite (395 g) in cyclohexane (3.5 L) at 0° C. was added portion wise pyridinium dichromate (794 g, 2110 mmol). To the mixture under nitrogen atmosphere was added dropwise tert-butyl hydroperoxide (355 mL, 2130 mmol) over a period of 10 min. The reaction mixture was warmed to 27° C. and was then stirred at that temperature for 48 h. Progress of the reaction was monitored by TLC (SiO₂, 30% Acetone/pet, Rf=0.4, UV-active). The reaction mixture was filtered and the filter cake was extracted with EtOAc (1000 mL). The filtrate was washed with saturated aq. Na₂S₂O₃ (2×500 mL); saturated aq. FeSO₄ (300 mL); and then brine (500 mL). The organic layer was dried over anhydrous Na₂SO₄, filtered and concentrated under reduced pressure to obtain the crude title compound (150 g).

### Preparation of ethyl 2-(3-(difluoromethyl)-4,4a-dihydrospiro[cyclopropa[3,4]cyclopenta[1,2-c]pyrazole-5,2′-[1,3]dithiolane]-1(3bH)-yl)acetate

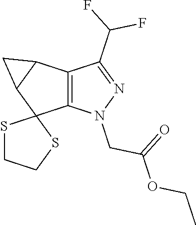 [SMILES: CCOC(=O)Cn1nc(C(F)F)c2c1C1(SCCS1)C1CC21]

To a stirred solution of ethyl 2-(3-(difluoromethyl)-5-oxo-3b,4,4a,5-tetrahydro-1H-cyclopropa[3,4]cyclopenta[1,2-c]pyrazol-1-yl)acetate (75 g, 269 mmol) in DCM (1500 mL) at 27° C. under nitrogen atmosphere was added ethane-1,2-dithiol (43.0 mL, 511 mmol) followed by the addition of boron trifluoride acetic acid (72.6 mL, 511 mmol). The solution was stirred for 16 h. Progress of the reaction was monitored by TLC (SiO₂, 20% Acetone/Pet, Rf=0.35, UV-Active). After completion, the reaction mixture was cooled to 0° C. and quenched via the addition of aq. saturated NaHCO₃ (500 mL). The mixture was extracted with DCM (2×1000 mL). The combined organics were washed with brine (1000 mL), dried over anhydrous Na₂SO₄, filtered and concentrated under reduced pressure to obtain a brown liquid. This material was subjected to silica gel column chromatography (Pet.:EtOAc 95:5→90:10) to afford ethyl 2-(3-(difluoromethyl)-4,4a-dihydrospiro[cyclopropa[3,4]cyclopenta[1,2-c]pyrazole-5,2′-[1,3]dithiolane]-1(3bH)-yl)acetate as an off-white solid, 80 g (74%). ¹H-NMR (400 MHz, CDCl₃) δ=6.61 (t, J=55.2 Hz, 1H), 5.00-4.85 (m, 2H), 4.29-4.19 (m, 2H), 3.55-3.46 (m, 4H), 2.63-2.53 (m, 1H), 2.49-2.38 (m, 1H), 1.30-1.24 (m, 4H), 0.65-0.60 (m, 1H). LCMS M+H=346.9.

### Preparation of ethyl 2-(3-(difluoromethyl)-5,5-difluoro-3b,4,4a,5-tetrahydro-1H-cyclopropa[3,4]cyclopenta[1,2-c]pyrazol-1-yl)acetate

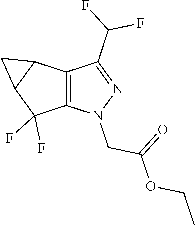 [SMILES: CCOC(=O)Cn1nc(C(F)F)c2c1C(F)(F)C1CC21]

To a stirred solution of 1,3-dibromo-5,5-dimethylimidazolidine-2,4-dione (26.3 g, 92 mmol) in DCM (20 mL) at −70° C. under N2 atmosphere was added HF-pyridine (2.460 g, 24.83 mmol). The solution was for 30 min. To the solution was added a solution of ethyl 2-(3-(difluoromethyl)-4,4a-dihydrospiro[cyclopropa[3,4]cyclopenta[1,2-c]pyrazole-5,2′-1,3]dithiolane]-1(3bH)-yl)acetate (10 g, 25 mmol) in DCM (20 mL). The reaction mixture was allowed to warm to −40° C. and then was stirred at that temperature for 1 h. Progress of the reaction was monitored by TLC (SiO₂, 30% EtOAc/Pet, Rf=0.3, UV in-active). The reaction mixture was quenched via the addition of aq. sat. NaHCO₃ (200 mL). The mixture was warmed to room temperature and was then extracted with EtOAc (2×100 mL). The combined organics were washed with brine (50 mL); dried over anhydrous Na₂SO₄; filtered; and were concentrated under reduced pressure to afford a brown solid. This material was subjected to silica gel column chromatography (Pet.:EtOAc 100:0→75-25) to afford ethyl 2-(3-(difluoromethyl)-5,5-difluoro-3b,4,4a,5-tetrahydro-1H-cyclopropa[3,4]cyclopenta[1,2-c]pyrazol-1-yl)acetate as a pale-yellow solid, 8.5 g (91%). ¹H NMR (400 MHz, CDCl₃) δ=6.62 (t, J=55.2 Hz, 1H), 4.82 (s, 2H), 4.30-4.18 (m, 2H), 2.51-2.37 (m, 2H), 1.42-1.35 (m, 1H), 1.31-1.23 (m, 3H), 1.14-1.08 (m, 1H). LCMS M+H=293.07.

### Preparation of 2-(3-(difluoromethyl)-5,5-difluoro-3b,4,4a,5-tetrahydro-1H-cyclopropa[3,4]cyclopenta[1,2-c]pyrazol-1-yl)acetic acid

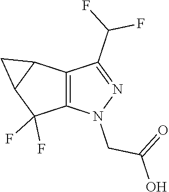 [SMILES: O=C(O)Cn1nc(C(F)F)c2c1C(F)(F)C1CC21]

To a stirred solution of ethyl 2-(3-(difluoromethyl)-5,5-difluoro-3b,4,4a,5-tetrahydro-1H-cyclopropa[3,4]cyclopenta[1,2-c]pyrazol-1-yl)acetate (15 g, 50 mmol) in THF (17 mL) and MeOH (66 mL) at 0° C. under N2 atmosphere was added a solution of LiOH (1.788 g, 74.7 mmol) in water (66 mL). The reaction mixture was allowed to warm to 27° C. and was then stirred for 3 h at that temperature. Progress of the reaction was monitored by TLC (SiO₂, 5% MeOH/DCM, Rf=0.2, UV Active). After completion, the reaction mixture was concentrated under reduced pressure; diluted with water (50 mL); and washed with EtOAc (2×250 mL) to remove impurities. The aqueous layer was adjusted to pH 2-3 using aq. HCl (1M), then was extracted with EtOAc (3×1000 mL). The combined organics were dried over anhydrous Na₂SO₄; filtered; and concentrated under reduced pressure to afford 2-(3-(difluoromethyl)-5,5-difluoro-3b,4,4a,5-tetrahydro-1H-cyclopropa[3,4]cyclopenta[1,2-c]pyrazol-1-yl)acetic acid as an off white solid, 14 g (98%). LCMS M+H=265.15.

### Separation Affording 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid and 2-((3bR,4aS)-3-(difluoromethyl)-5,5-difluoro-3b,4,4a,5-tetrahydro-1H-cyclopropa[3,4]cyclopenta[1,2-c]pyrazol-1-yl)acetic acid

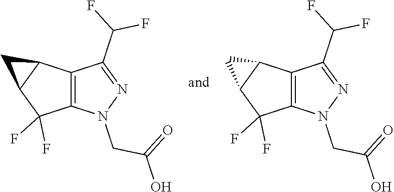 [SMILES: O=C(O)Cn1nc(C(F)F)c2c1C(F)(F)[C@H]1C[C@@H]21 ; O=C(O)Cn1nc(C(F)F)c2c1C(F)(F)[C@@H]1C[C@H]21]

2-(3-(difluoromethyl)-5,5-difluoro-3b,4,4a,5-tetrahydro-1H-cyclopropa[3,4]cyclopenta[1,2-c]pyrazol-1-yl)acetic acid (5.5 g) was dissolved in isopropanol (20 mL). The solution was subjected portion-wise to SFC chiral separation as follows: Instrument=Thar 80; column=Chiralpak IC 30×250 mm, 5 micron; solvent A=super critical C₀₂; solvent B=isopropanol with 0.5% isopropyl amine (v/v); eluent composition=70% A:30% B; flow-rate=65 g/min; back-pressure=100 bar; temperature=30° C.; injection volume=2.5 mL; detection=220 nm. 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid was collected as peak eluting from 7.5 min. to 14 min; 2-((3bR,4aS)-3-(difluoromethyl)-5,5-difluoro-3b,4,4a,5-tetrahydro-1H-cyclopropa[3,4]cyclopenta[1,2-c]pyrazol-1-yl)acetic acid was collected as a peak eluting from 2.7 min. to 5.8 min. For each enantiomer, the resulting solution was concentrated under reduced pressure and the resulting solids were dissolved in EtOAc, then twice washed with aq. citric acid (1M) followed by water followed by brine. The organic solution was dried over Na₂SO₄; filtered; then concentrated in vacuo to afford the separated enantiomer in 80-90% recovery.

### Preparation of N-(7-amino-4-chloro-1-methyl-1H-indazol-3-yl)-N-(4-methoxybenzyl)methanesulfonamide

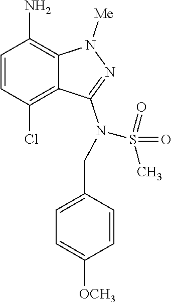 [SMILES: COc1ccc(CN(c2nn(C)c3c(N)ccc(Cl)c23)S(C)(=O)=O)cc1]

### Synthesis Scheme:

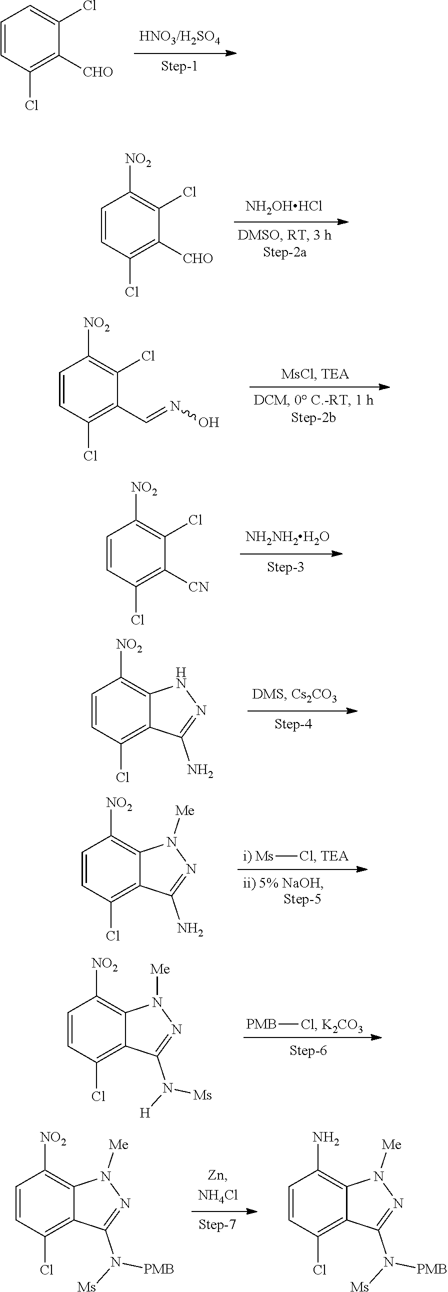

### Step 1: Preparation of 2,6-dichloro-3-nitrobenzaldehyde

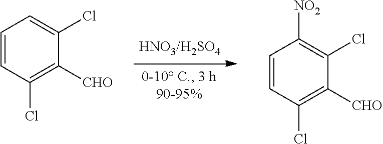 [SMILES: O=Cc1c(Cl)cccc1Cl ; O=Cc1c(Cl)ccc([N+](=O)[O-])c1Cl]

To a solution of sulfuric acid (H₂SO₄) (5.63 L, 4.5 V) in a round-bottom flask at 0-5° C. was added 2,6-dichlorobenzaldehyde (1.25 kg, 7.10 mol, 1.0 equiv.) in portions at below 15° C. The reaction mass was stirred at 0-5° C. for 30 min. A solution of freshly prepared nitration mixture [Prepared from Conc. H₂SO₄ (0.425 L, 0.34 V) and 70% HNO₃ (0.85 kg, 13.49 mol, 1.30 equiv.) at 0° C.] was added to the above reaction mixture at below 10° C. [Note: Reaction is slightly exothermic (3-6° C.); so that addition is preferred at lower temperature]. The reaction mixture was stirred at 5-10° C. for 2-3 h. After completion of the reaction (monitored by TLC), it was quenched with ice cold water (18.75 L, 15 V) at below 25° C. Then the reaction mass was allowed warm to room temperature and stirred for 2 h. The solids were isolated by filtration and then were washed with water (2.5 L, 2.0 V). Bulk residual water was removed from the solids by maintaining vacuum filtration for 60-90 min. The crude wet solid was initially dried under air atmosphere; then in a hot air oven at 50-55° C. for 10-12 h (until moisture content is not more than 5.0%) to get the dried title product, 2,6-dichloro-3-nitrobenzaldehyde (1.44 kg, 92% yield) as a yellow solid. ¹H NMR (400 MHz, CDCl₃): δ10.44 (s, 1H), 7.88 (d, J=8.4 Hz, 1H), 7.56 (d, J=8.8 Hz, 1H).

### Step 2: Preparation of 2,6-dichloro-3-nitrobenzonitrile

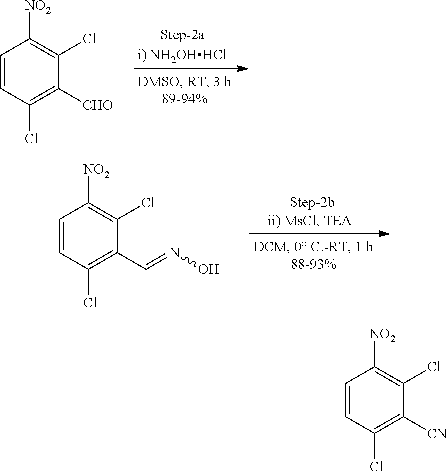

(Step-2a) To a solution of DMSO (5.9 L, 5.0 V)) in a round-bottom flask was added 2,6-dichloro-3-nitrobenzaldehyde (1.17 kg, 5.31 mol, 1.0 equiv.) at room temperature. After being stirred for 30 min at room temperature, hydroxylamine hydrochloride (0.63 kg, 9.04 mol, 1.70 equiv.) was added and the reaction mass was stirred at room temperature for 3 h. After completion of the reaction (monitored by TLC), the reaction mass was quenched by the addition of ice-cold water (18.0 L, 15.0 V) added at a rate sufficient to maintain the temperature below 30° C. (Observation: Solids formed upon water addition). The reaction mass was stirred at room temperature for 60-90 min. The solids were isolated by filtration; washed with water (2.5 L, 2.0 V); followed by washing with a mixture of acetone and hexanes (6.0 L, 1:1 ratio). Bulk residual water was removed from the solids by maintaining vacuum filtration for 60-90 min. The wet solid was initially air dried and then finally dried in a hot air oven at 50-55° C. for 10-12 h (until moisture content was not more than 1.0%) to get the dried target product, 2,6-dichloro-3-nitrobenzaldehyde oxime (1.22 kg, 92% yield) as an off-white solid. The crude product (which contains 10-20% of 2,6-dichloro-3-nitrobenzonitrile) was used directly in the next step without further purification.
(Step-2b) To a stirred solution of the crude oxime (preparation described above, 1.13 kg, 4.80 mol, 1.0 equiv.) in DCM (9.04 L, 8.0 V) at 0-5° C. was added triethylamine (“TEA”, 1.02 kg, 10.09 mol, 2.1 equiv.). After being stirred for 5 min, methanesulfonyl chloride (0.60 kg, 5.29 mol, 1.1 equiv.) was added (Observation: An exotherm is noted during the addition) slowly at 15° C. Then the reaction mass was stirred at room temperature for 30-45 min. After completion of the reaction (progress of reaction was monitored by TLC; mobile phase: 20% ethyl acetate in hexanes), the reaction mass was diluted with water (6.78 L, 6.0 V); the organic layer was separated; and the aqueous layer was extracted with DCM (3.4 L, 3.0 V). The combined organic layers were washed with brine (5.65 L, 5.0 V); dried over Na₂SO₄; and concentrated under vacuum. The resulting crude solids were triturated with hexanes (4.50 L, 4.0 V) at room temperature. The wet material was dried in a hot air oven at 50-55° C. for 5-6 h to get the dried product, 2,6-dichloro-3-nitrobenzonitrile (0.95 kg, 91% yield) as a yellow solid. ¹H NMR (400 MHz, CDCl₃): δ8.07 (d, J=8.8 Hz, 1H), 7.63 (d, J=8.8 Hz, 1H).

### Step 3: Preparation of 4-chloro-7-nitro-1H-indazol-3-amine

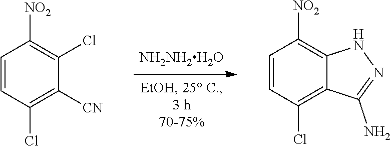

To a stirred solution of 2,6-dichloro-3-nitrobenzonitrile (750.0 g, 3.45 mol, 1.0 equiv.) in ethanol (7.5 L, 10.0 V) at 15-20° C. was slowly added hydrazine hydrate (519.0 g, 10.36 mol, 3.0 equiv.) while maintaining the reaction mass below 25° C. (Observation: Addition is slightly exothermic and solid formation will begin upon addition). The reaction mixture temperature was slowly raised to room temperature and then the mixture was stirred for 3 h (Observation: the quantity of solids will increase during this time). After completion of the reaction (monitored by TLC), the mixture was diluted with water (7.5 L, 10.0 V) and further stirred for 1 h at room temperature. The solids were isolated via filtration and then were washed with water (2.25 L, 3.0 V). The wet solid was washed with a 1:1 ratio mixture of acetone (1.875 L, 2.5 V) and hexanes (1.875 L, 2.5 V). Bulk residual water was removed from the solids by maintaining vacuum filtration for 60-90 min. The wet solid was finally dried in a hot air oven for 7-8 h at 50° C. (until moisture content reaches below 1.5%) to get the dried product, 4-chloro-7-nitro-1H-indazol-3-amine (549.0 g, 75% yield) as a brick red-colored solid. ¹H NMR (400 MHz, CDCl₃): δ10.36 (bs, 1H), 8.20 (d, J=8.4 Hz, 1H), 7.07 (d, J=8.40 Hz, 1H), 4.73 (bs, 2H).

### Step 4: Preparation of 4-chloro-1-methyl-7-nitro-1H-indazol-3-amine

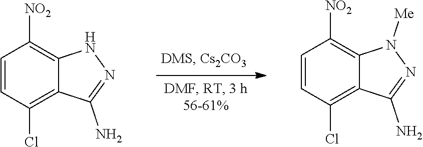

To a stirred solution of 4-chloro-7-nitro-1H-indazol-3-amine (500 g, 0.42 mol, 1.0 equiv.) in DMF (5.0 L, 10.0 V) at 5-10° C. was slowly added cesium carbonate (Cs₂CO₃) (1.91 kg, 5.88 mol, 2.5 equiv.) while maintaining the reaction mass below 10° C. After being stirred for 5-10 min, dimethyl sulphate (326.3 g, 2.59 mol, 1.1 equiv.) was added while maintaining the reaction mass below 10° C. (Note: Slow addition is preferred for obtaining more favorable regio-selectivity). Then, the reaction temperature was slowly raised to room temperature and stirring was continued an additional 2 h at the same temperature. After completion of the reaction (monitored by TLC), the reaction mass was quenched by the addition of ice-cold water (15.0 L, 30.0 V) and the resulting mixture was then stirred for 6-8 h at room temperature. The solids were isolated via filtration and were then washed with water (1.5 L, 3.0 V). The wet solid was washed with IPA (1.5 L, 3.0 V) followed by hexanes (1.0 L, 2.0 V). Bulk residual water was removed from the solids by maintaining vacuum filtration for 60-90 min. The wet solid was dried in a hot air oven for 7-8 h at 50° C. (until moisture content is below 1.0%). The isolated material, 4-chloro-1-methyl-7-nitro-1H-indazol-3-amine (319.0 g, 60% yield), was used in the next step without further purification. ¹H NMR (400 MHz, CDCl₃): δ7.97 (d, J=8.32 Hz, 1H), 6.97 (d, J=8.24 Hz, 1H), 4.63 (bs, 2H), 3.96 (s, 3H).

### Step 5: Preparation of N-(4-chloro-1-methyl-7-nitro-1H-indazol-3-yl)methanesulfonamide

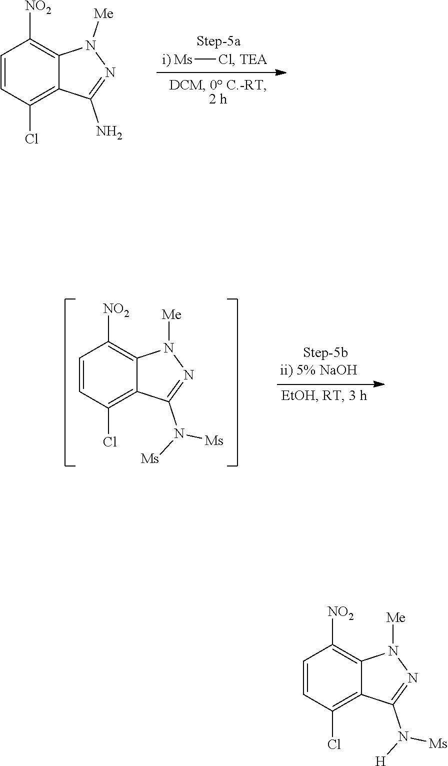

(Step 5a) To a solution of 4-chloro-1-methyl-7-nitro-1H-indazol-3-amine (625.0 g, 2.76 mol, 1.0 equiv.) in DCM (6.25 L, 10.0 V) at 0-5° C. was added triethylamine (TEA) (837.0 g, 8.27 mol, 3.0 equiv.); followed by the addition of 4-dimethylaminopyridine (DMAP) (20.60 g, 0.165 mol, 0.06 equiv.). The reaction mass was stirred for 5-10 min., then methanesulfonyl chloride (MsCl) (790.0 g, 6.89 mol, 2.5 equiv.) added slowly while maintaining the reaction mass below 10° C. The reaction mixture was allowed to warm to room temperature and was then stirred for 1.5-2.0 h. After completion of the reaction (monitored by TLC), the mixture was diluted with water (6.25 L, 10.0 V) and then stirred at room temperature for 15 min. The organic layer was separated, and the aqueous layer was extracted with DCM (6.25 L, 10.0 V). The combined organic layers were washed with brine (1.25 L, 2.0 V), dried over Na₂SO₄ and concentrated to get the crude solids. The solids were triturated with hexanes (1.25 L, 2.0 V) at room temperature to obtain the intermediate, N-(4-chloro-1-methyl-7-nitro-1H-indazol-3-yl)-N-(methylsulfonyl)methanesulfonamide, which was used directly in the next step.

(ii) To a stirred solution of N-(4-chloro-1-methyl-7-nitro-1H-indazol-3-yl)-N-(methylsulfonyl)methanesulfonamide (prepared above) in ethanol (10.5 L, 20.0 V) at room temperature was added slowly an aq. 5% NaOH solution (4.38 L, 7.0 V) [Note: Slow addition is preferred via dropping funnel]. The reaction mass was stirred at the same temperature for 3 h. After completion of the reaction (monitored by TLC) [Sample preparation for TLC analysis: ˜1.0 ml of sample acidified with aq. 2.0 N HCl to reach the pH: 2-3, extract it with ethyl acetate and analyze the organic layer by TLC], the reaction mass was cooled to 0-5° C. and the pH was adjusted to 2-3 by the addition of aq. 2.0 N HCl (3.13 L, 5.0 V) while maintain the reaction temperature below 10° C. [Note: Precipitation occurred upon addition of HCl and increased with stirring]. The reaction mixture was warmed to room temperature and then stirred for 1.5-2.0 h. Solids obtained were isolated via filtration and were then washed with water (1.25 L, 2.0 V); followed by washing with hexanes (1.25 L, 2.0 V). Bulk residual water was removed from the solids by maintaining vacuum filtration for 60-90 min. The wet material was dried in a hot air oven at 50° C. for 6-7 h (Until the moisture content is below 1.0%) to get the dried product, N-(4-chloro-1-methyl-7-nitro-1H-indazol-3-yl)methanesulfonamide (640.0 g, 76%) as a yellow solid. ¹H NMR (400 MHz, CDCl₃): δ8.05 (d, J=8.32 Hz, 1H), 7.32 (bs, 1H), 7.17 (d, J=8.28 Hz, 1H), 4.15 (s, 3H), 3.45 (s, 3H).

### Step 6: Preparation of N-(4-chloro-1-methyl-7-nitro-1H-indazol-3-yl)-N-(4-methoxybenzyl)methanesulfonamide

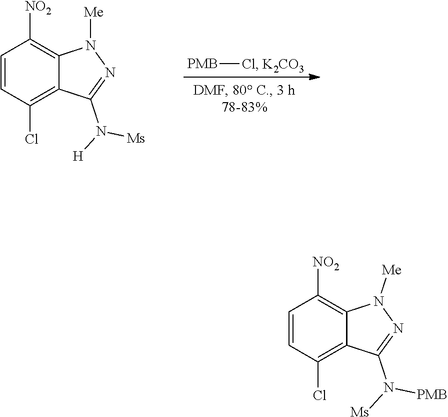

To a mixture of N-(4-chloro-1-methyl-7-nitro-1H-indazol-3-yl)methanesulfonamide (635.0 g, 2.08 mol, 1.0 equiv.) and 1-(chloromethyl)-4-methoxybenzene (359.0 g, 2.30 mol, 1.1 equiv.) in DMF (6.35 L, 10.0 V) at room temperature was added potassium carbonate (374.7 g, 2.70 mol, 1.3 equiv.). The reaction mixture was heated to 80-90° C. and maintained at that temperature for 3 h. After completion of the reaction (monitored by TLC), the mixture was poured into ice cold water (19.05 L, 30.0 V) [Note: Slow quenching with vigorous stirring is preferred to avoid clumping as the product precipitates]. The resulting solids were isolated via filtration and washed with water (1.90 L, 3.0 V); then the solids were washed with hexanes (1.27 L, 2.0 V). Bulk residual water was removed from the solids by maintaining vacuum filtration for 60-90 min. The isolated solid was dissolved in Ethyl acetate (12.7 L, 20.0 V) and charcoal was added (63.5 g). The mixture was heated to 60-70° C. and then stirred for 30-45 min. at that temperature. The mixture was filtered while still hot (40-50° C.) through a pad of Celite and the Celite pad was then extracted with ethyl acetate (3.17 L, 5.0 V). The combined filtrates were concentrated to dryness under reduced pressure at below 50° C. Ethyl acetate (0.635 L, 1.0 V) was added to the solids at room temperature. The resultant solid suspension was stirred for 30 min. The solids were isolated via filtration and then were washed with hexanes (1.27 L, 2.0 V). Residual water was removed from the solids by maintaining vacuum filtration for 45-60 min. to afford the product N-(4-chloro-1-methyl-7-nitro-1H-indazol-3-yl)-N-(4-methoxybenzyl) methane sulfonamide (705.0 g, 80% yield) as a yellow solid. ¹H NMR (400 MHz, CDCl₃): δ7.99 (d, J=8.24 Hz, 1H), 7.27 (d, J=8.68 Hz, 2H), 7.19 (d, J=8.24 Hz, 1H), 6.80 (d, J=8.44 Hz, 2H), 4.95-4.76 (m, 2H), 4.17 (s, 3H), 3.76 (s, 3H), 3.01 (s, 3H).

### Step 7: Preparation of N-(7-Amino-4-chloro-1-methyl-1H-indazol-3-yl)-N-(4-methoxybenzyl)methanesulfonamide

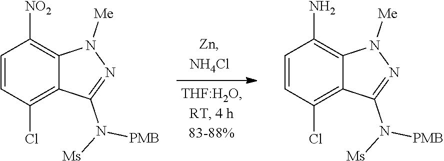

To a stirred suspension of zinc powder (540.0 g, 8.23 mol, 10.0 equiv.) in a mixture of THF (3.50 L, 10.0 V) and water (7.0 L, 20.0 V) at room temperature was added ammonium chloride (NH₄Cl) (449.0 g, 8.23 mol, 10.0 equiv.). To the mixture was added N-(4-chloro-1-methyl-7-nitro-1H-indazol-3-yl)-1(4-methoxybenzyl)methanesulfonamide (350 g, 0.823 mol, 1.0 equiv.) in THF (7.0 L, 20.0 V). The reaction mixture was stirred at room temperature for 3-4 h. After completion of the reaction (monitored by in-process TLC/HPLC), the mixture was diluted with ethyl acetate (3.5 L, 10.0 V) and water (1.12 L, 2.5 V). The mixture was stirred for 15 min. The reaction mass was filtered through a pad of Celite bed washing with ethyl acetate (1.75 L, 5.0 V). The bi-phasic filtrate was collected, and the phases were separated. The aqueous layer was extracted with ethyl acetate (3.50 L, 10.0 V). The combined organic layers were washed with brine (3.50 L, 10 V), dried over Na₂SO₄, and then concentrated in vacuo to afford a crude solid. To the crude product was added MTBE (3.25 L, 10 V) and the suspension was stirred for 30 min at room temperature. The solids were isolated by filtration. Bulk residual water was removed from the solids by maintaining vacuum filtration for 30-45 min. The wet product was dried in a hot air oven (50° C.) for 2 h to afford the title product, N-(7-amino-4-chloro-1-methyl-1H-indazol-3-yl)-1(4-methoxybenzyl)methanesulfonamide (276.0 g, 85% yield) as off-white solid. ¹H NMR (400 MHz, CDCl₃): δ7.29-7.26 (m, 2H), 6.86-6.79 (m, 2H), 6.42 (d, J=7.80 Hz, 1H), 4.99-4.70 (m, 2H), 4.25 (s, 3H), 3.77 (s, 5H), 2.98 (s, 3H).

### Preparation of tert-butyl(S)-(1-(7-bromo-3-(4-chloro-3-(N-(4-methoxybenzyl)methylsulfonamido)-1-methyl-1H-indazol-7-yl)-4-oxo-3,4-dihydroquinazolin-2-yl)-2-(3,5-difluorophenyl)ethyl)carbamate

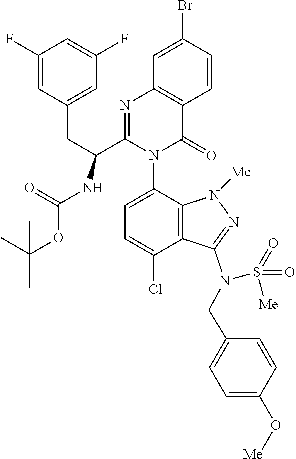 [SMILES: COc1ccc(CN(c2nn(C)c3c(-n4c([C@H](Cc5cc(F)cc(F)c5)NC(=O)OC(C)(C)C)nc5cc(Br)ccc5c4=O)ccc(Cl)c23)S(C)(=O)=O)cc1]

To a solution of (S)-2-((tert-butoxycarbonyl)amino)-3-(3,5-difluorophenyl)propanoic acid (3.82 g, 12.66 mmol), 2-amino-4-bromobenzoic acid (3.01 g, 13.93 mmol) and N-(7-amino-4-chloro-1-methyl-1H-indazol-3-yl)-N-(4-methoxybenzyl)methanesulfonamide (5 g, 12.66 mmol) in pyridine (50 mL) was added diphenyl phosphite (9.80 mL, 50.6 mmol). The resulting mixture was placed on a preheated oil bath (70° C.) and heated at 70° C. for 16 h. The mixture was cooled to room temperature and then concentrated under reduced pressure. The mixture was then diluted with EtOAc (approximately 500 mL) and washed with aqueous citric acid (0.5M, 2×50 mL), then aqueous NaOH (1M, 3×50 mL), dried over Na₂SO₄, filtered, and concentrated. The residue was then purified via silica gel chromatography (330 g silica gel column, gradient of hexanes:EtOAc 0:100-450:50) to afford tert-butyl (S)-(1-(7-bromo-3-(4-chloro-3-(N-(4-methoxybenzyl)methylsulfonamido)-1-methyl-H-indazol-7-yl)-4-oxo-3,4-dihydroquinazolin-2-yl)-2-(3,5-difluorophenyl)ethyl)carbamate (6.2 g, 7.22 mmol, 57.1% yield) as pale yellow solid foam (inseparable mixture of atropisomers). LC/MS: m/z=801.10 [M-tBu].

### Preparation of (S)—N-((6P)-7-(2-(1-amino-2-(3,5-difluorophenyl)ethyl)-7-bromo-4-oxoquinazolin-3(4H)-yl)-4-chloro-1-methyl-1H-indazol-3-yl)methanesulfonamide

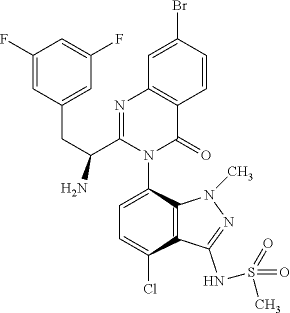

To a stirred solution of tert-butyl (S)-(1-(7-bromo-3-(4-chloro-3-(N-(4-methoxybenzyl)methylsulfonamido)-1-methyl-1H-indazol-7-yl)-4-oxo-3,4-dihydroquinazolin-2-yl)-2-(3,5-difluorophenyl)ethyl)carbamate (6.2 g, 7.22 mmol) in dichloromethane (DCM) (50 mL) was added trifluoroacetic acid (20 mL, 260 mmol) followed by trifluoromethanesulfonic acid (0.770 mL, 8.67 mmol). The resulting dark red solution was stirred at room temperature for 1 h. LCMS at this point indicates two peaks containing the desired product mass, consistent with the presence of two diastereomeric atropisomers (ratio of approximately 30:70). The mixture was concentrated in vacuo and the resulting residue was partitioned between EtOAc (300 mL) and aq. NaOH (1M, 30 mL). The aq. phase was tested and determined to be pH>=8.0. The organic phase was isolated and dried over Na₂SO₄, filtered, and then concentrated in vacuo. The residue was purified in three approximately equal portions via C18 chromatography (275 g RediSep Gold Column, Mobile Phase A: 5:95 acetonitrile:water with 0.1% TFA; Mobile Phase B: 95:5 acetonitrile:water with 0.1% TFA; gradient of 10-60% B over 30 min). Fractions containing the major atropisomer (second eluting) were combined, adjusted to pH 8 via addition of aq. 1M NaOH; extracted with ethyl acetate; washed with brine (sat. aq. NaCl); dried over Na₂SO₄; filtered; and then concentrated to afford the desired major atropisomer (S)—N-((6P)-7-(2-(1-amino-2-(3,5-difluorophenyl)ethyl)-7-bromo-4-oxoquinazolin-3(4H)-yl)-4-chloro-1-methyl-1H-indazol-3-yl)methanesulfonamide (2.4 g, 3.76 mmol, 52% yield). ¹H NMR (500 MHz, DMSO-d₆) δ ppm 8.11 (d, J=8.55 Hz, 1H), 8.06 (d, J=1.53 Hz, 1H), 7.81 (dd, J=8.55, 1.83 Hz, 1H), 7.33 (s, 2H), 6.96-7.05 (m, 1H), 6.75 (br d, J=7.02 Hz, 2H), 3.67 (s, 3H), 3.56 (dd, J=7.63, 5.19 Hz, 1H), 3.25-3.29 (m, 1H), 3.21 (s, 3H), 2.81 (dd, J=13.43, 8.24 Hz, 1H). LCMS: m/z=637.05 [M+H]⁺.

### Preparation of N—((S)-1-((3P)-7-bromo-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

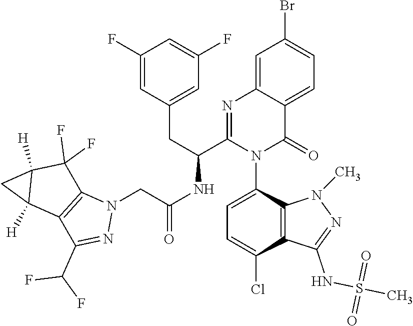

To a solution of (S)—N-((6P)-7-(2-(1-amino-2-(3,5-difluorophenyl)ethyl)-7-bromo-4-oxoquinazolin-3(4H)-yl)-4-chloro-1-methyl-1H-indazol-3-yl)methanesulfonamide (2.08 g, 3.26 mmol), 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.861 g, 3.26 mmol) and diisopropylethylamine (“DIPEA”) (1.709 mL, 9.78 mmol) in tetrahydrofuran (THF) (30 mL) was added HATU (1.364 g, 3.59 mmol). The resulting mixture was stirred at room temp for 3 h. To the mixture was added ammonia in methanol (2M, 3 mL). The mixture was stirred at room temp for 30 min. Water was then added and the mixture was extracted with ethyl acetate; washed with brine; dried over Na₂SO₄, filtered; and concentrated in vacuo. The resulting residue was subjected to silica gel chromatography (hexanes:EtOAc 100:0-430:70) to afford N—((S)-1-((3P)-7-bromo-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (2.5 g, 2.83 mmol, 87% yield). ¹H NMR (500 MHz, CDCl₃) δ ppm 8.18 (d, J=8.24 Hz, 1H), 7.88 (d, J=1.53 Hz, 1H), 7.72 (dd, J=8.55, 1.83 Hz, 1H), 7.33 (s, 1H), 7.16 (d, J=7.63 Hz, 1H), 6.57-6.83 (m, 4H), 6.38 (br d, J=5.80 Hz, 2H), 4.71-4.80 (m, 1H), 4.63 (d, J=6.71 Hz, 2H), 3.56 (s, 3H), 3.40 (s, 3H), 3.18 (dd, J=13.73, 6.10 Hz, 1H), 2.86 (dd, J=13.58, 7.48 Hz, 1H), 2.52-2.61 (m, 1H), 2.41-2.50 (m, 1H), 1.42-1.50 (m, 1H), 1.09-1.16 (m, 1H). LCMS: m/z=883.05 [M+H]⁺.

### Preparation of N—((S)-1-((3P)-3-(4-chloro-1-methyl-3-(methylsulfonamido)-1H-indazol-7-yl)-4-oxo-7-(4,4,5,5-tetramethyl-1,3,2-dioxaborolan-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

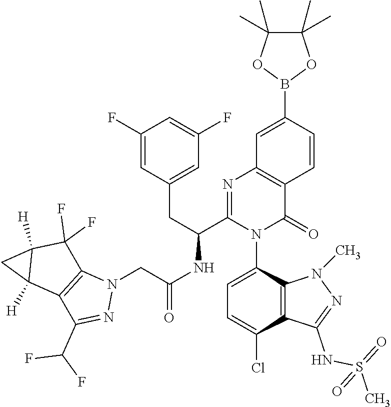

To a round bottom flask equipped with a stir bar was added N—((S)-1-((3P)-7-bromo-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1.00 g, 1.13 mmol), 4,4,4′,4′,5,5,5′,5′-octamethyl-2,2′-bi(1,3,2-dioxaborolane) (431 mg, 1.70 mmol), potassium acetate (333 mg, 3.39 mmol), and [1,1′-bis(diphenylphosphino)ferrocene]dichloropalladium(II) (“Pd(dppf)Cl₂”) (83 mg, 0.113 mmol). The flask was sealed with a rubber septum, and then was placed under an argon atmosphere. To the flask was added dioxane (23 mL). The reaction mixture was degassed with argon, then the reaction mixture was stirred at 60° C. for 16 h. The reaction mixture was concentrated in vacuo and adsorbed onto Celite. The resulting powder was subjected to silica gel chromatography (hexanes:EtOAc 100:0-40:100 over 10 column volumes) to afford N—((S)-1-((3P)-3-(4-chloro-1-methyl-3-(methylsulfonamido)-1H-indazol-7-yl)-4-oxo-7-(4,4,5,5-tetramethyl-1,3,2-dioxaborolan-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1.2 g, quantitative yield). LCMS: During LCMS analysis both the boronic acid and boronate were observed. Conditions: Wavelength1: 220 nm, Wavelength2: 254 nm, Injection Vol.: 5.00 μl, Stop Time: 4.00, Grad. Time: 3.0, Start % B: 0, End % B: 100, Total Flow: 0.80 ml/min, Solvent A: 95:5 Water:MeCN 0.1% TFA, Solvent B: 5:95 Water:MeCN 0.1% TFA, Column: Acquity UPLC BEH C18 1.7 um; Result: retention time (boronic acid): 2.112 min., mass found: 849.15 (M+H); retention time (boronic ester): 2.733 min., mass found: 931.25 (M+H). ¹H NMR (CDCl₃, 500 MHz) δ 8.26 (d, 1H, J=7.6 Hz), 8.11 (s, 1H), 7.95 (d, 1H, J=7.6 Hz), 7.3-7.3 (m, 1H), 7.14 (d, 1H, J=7.9 Hz), 6.7-6.7 (m, 3H), 6.35 (d, 2H, J=6.8 Hz), 4.7-4.8 (m, 1H), 4.1-4.2 (m, 1H), 3.70 (s, 1H), 3.47 (s, 3H), 3.37 (s, 3H), 3.1-3.2 (m, 1H), 2.8-2.9 (m, 1H), 2.6-2.7 (m, 1H), 2.3-2.5 (m, 1H), 1.8-1.9 (m, 2H), 1.24 (s, 12H), 1.1-1.2 (m, 1H)

### Preparation of N—((S)-1-((3P)-7-bromo-3-(4-chloro-3-(N-(4-methoxybenzyl)methylsulfonamido)-1-methyl-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

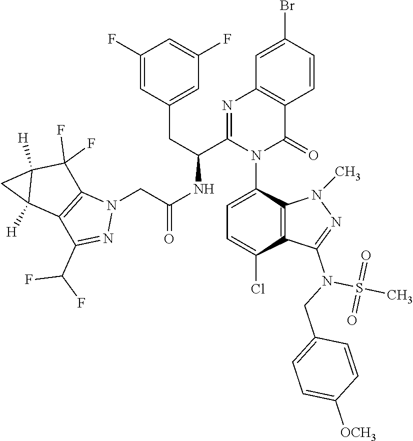

1-(chloromethyl)-4-methoxybenzene (0.276 mL, 2.036 mmol) was added to a stirred solution of N—((S)-1-((3P)-7-bromo-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1.5 g, 1.697 mmol) and cesium carbonate (0.553 g, 1.697 mmol) in N,N-Dimethylformamide (DMF) (10 mL), and the resulting mixture was stirred at room temp for 16 h. Water was then added and the mixture was extracted with ethyl acetate, washed with brine, dried (Na₂SO₄), filtered and concentrated. The residue was then subjected to silica gel column chromatography (hexanes:EtOAc 95:5-470:30) to afford N—((S)-1-((3P)-7-bromo-3-(4-chloro-3-(N-(4-methoxybenzyl)methylsulfonamido)-1-methyl-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide, 1.4 g (82%). LCMS analysis conditions: Wavelength1: 220 nm; Wavelength2: 254 nm; Injection Vol.: 5.00 μl; Stop Time: 4.50 min; Grad. Time: 3.50 min; Start % B: 0; End % B: 100; Total Flow: 0.80 ml/min; Solvent A: 95:5 Water:MeCN with 0.1% TFA; Solvent B: 5:95 Water:MeCN with 0.1% TFA; Column=Acquity UPLC BEH C18, 2.1×100 mm, 1.7 μm. LCMS analysis result: retention time: 3.536 min, M+H: 1003.05.

### Preparation of N—((S)-1-((3P)-3-(4-chloro-3-(N-(4-methoxybenzyl)methylsulfonamido)-1-methyl-1H-indazol-7-yl)-4-oxo-7-(1H-pyrazol-3-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

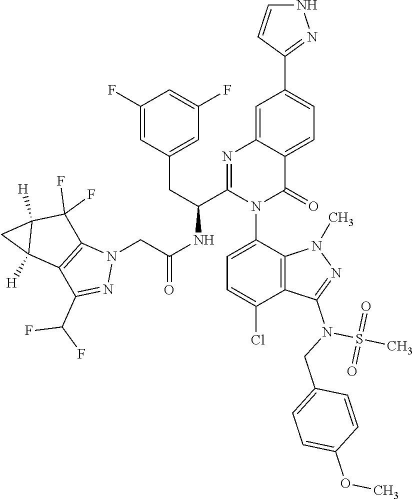 [SMILES: COc1ccc(CN(c2nn(C)c3c(-n4c([C@H](Cc5cc(F)cc(F)c5)NC(=O)Cn5nc(C(F)F)c6c5C(F)(F)[C@@H]5C[C@H]65)nc5cc(-c6cc[nH]n6)ccc5c4=O)ccc(Cl)c23)S(C)(=O)=O)cc1]

To a solution of N—((S)-1-((3P)-7-bromo-3-(4-chloro-3-(N-(4-methoxybenzyl)methylsulfonamido)-1-methyl-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (340 mg, 0.339 mmol), (1H-pyrazol-3-yl)boronic acid (114 mg, 1.016 mmol) and K₃PO₄ (216 mg, 1.016 mmol) in Tetrahydrofuran (THF) (5 mL)/Water (1.250 mL) was added Dichloro[9,9-dimethyl-4,5-bis(diphenylphosphino)xanthene]palladium(II) (25.6 mg, 0.034 mmol) and the resulting mixture was stirred at 60° C. for 3 h. LCMS at this point showed a peak with the expected M+H. To the mixture was added water the mixture was extracted with ethyl acetate. The organic phase was dried (Na₂SO₄), filtered, and the filtrate concentrated in vacuo. The residue was subjected to silica gel chromatography (5-100% EtOAC in hexanes) to afford N—((S)-1-((3P)-3-(4-chloro-3-(N-(4-methoxybenzyl)methylsulfonamido)-1-methyl-1H-indazol-7-yl)-4-oxo-7-(1H-pyrazol-3-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (260 mg, 0.262 mmol, 77% yield). LCMS (M+H)+=991.05

### Preparation of Example 1: N—((S)-1-((3P)-3-(4-chloro-1-methyl-3-(methylsulfonamido)-1H-indazol-7-yl)-4-oxo-7-(2-(trifluoromethyl)thiazol-4-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

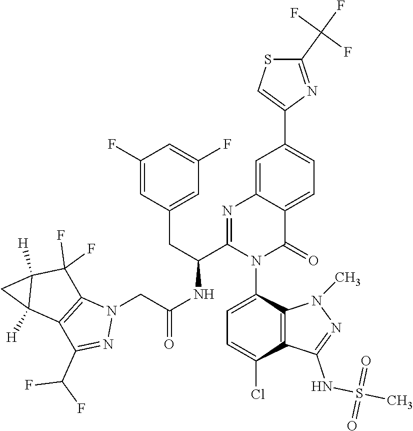

To a 1 dram vial charged with N—((S)-1-((3P)-7-bromo-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (35 mg, 0.040 mmol), 4-(4,4,5,5-tetramethyl-1,3,2-dioxaborolan-2-yl)-2-(trifluoromethyl)thiazole (22.10 mg, 0.079 mmol), Dichloro[9,9-dimethyl-4,5-bis(diphenylphosphino)xanthene]palladium(II) (2.99 mg, 3.96 μmol) and K₃PO₄ (25.2 mg, 0.119 mmol) was added degassed (Nitrogen bubbling for 1 min) Tetrahydrofuran (THF) (1 mL)/Water (0.25 mL) and the resulting mixture was stirred at room temp for 16 h under an atmosphere of nitrogen. The LCMS indicated the reaction was complete. The reaction mass was transferred to a 20 mL scintillation vial. To the reaction was added EtOAc (5 mL) and aqueous 1 M HCl (5 mL). The vial was sealed and shaken. The organic layer was pipetted away and concentrated. The residue was subjected to HPLC purification to afford the title compound, N—((S)-1-((3P)-3-(4-chloro-1-methyl-3-(methylsulfonamido)-1H-indazol-7-yl)-4-oxo-7-(2-(trifluoromethyl)thiazol-4-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.6 min.; observed ion=954.1 (M−H). 1H NMR (500 MHz, METHANOL-d4) δ ppm 8.59-8.63 (m, 1H), 8.54-8.56 (m, 1H), 8.39 (d, J=8.35 Hz, 1H), 8.28 (dd, J=8.35, 1.79 Hz, 1H), 7.32 (d, J=8.05 Hz, 1H), 7.23 (d, J=7.75 Hz, 1H), 6.58-6.82 (m, 4H), 4.54 (d, J=4.47 Hz, 2H), 3.64 (s, 3H), 3.50 (dd, J=14.01, 5.07 Hz, 1H), 3.26 (s, 3H), 3.13 (dd, J=14.01, 9.24 Hz, 1H), 2.39-2.49 (m, 2H), 1.34-1.39 (m, 1H), 0.99-1.04 (m, 1H)

### Preparation of Example 2: N—((S)-1-((3P)-3-(4-chloro-1-methyl-3-(methylsulfonamido)-1H-indazol-7-yl)-7-(4-(methylsulfonyl)phenyl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

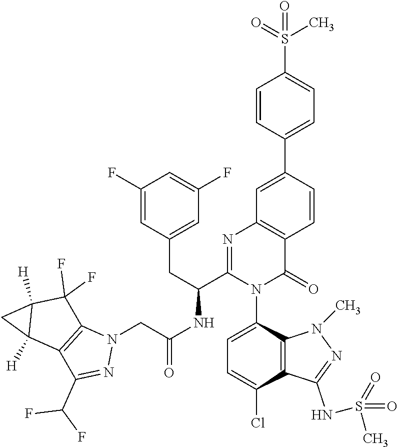

The title compound was prepared according to General Procedure K using 1-bromo-4-(methylsulfonyl)benzene as the coupling partner. The experiment afforded the title compound, N—((S)-1-((3P)-3-(4-chloro-1-methyl-3-(methylsulfonamido)-1H-indazol-7-yl)-7-(4-(methylsulfonyl)phenyl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method C: retention time=2.34 min.; observed ion=959.3 (M+H). 1H NMR (METHANOL-d4, 500 MHz) δ 8.67 (br d, 1H, J=8.5 Hz), 8.31 (br d, 1H, J=8.5 Hz), 8.0-8.1 (m, 3H), 8.0-8.0 (m, 2H), 7.90 (br d, 1H, J=8.5 Hz), 7.21 (br d, 1H, J=7.0 Hz), 7.10 (br d, 1H, J=7.9 Hz), 6.7-6.7 (m, 1H), 6.58 (s, 1H), 6.52 (br d, 2H, J=7.3 Hz), 6.47 (s, 1H), 4.42 (s, 2H), 3.52 (s, 3H), 3.3-3.4 (m, 1H), 3.13 (br d, 6H, J=17.7 Hz), 3.0-3.0 (m, 1H), 2.3-2.3 (m, 2H), 1.2-1.3 (m, 2H), 0.9-0.9 (m, 1H)

### Preparation of Example 3: 2-((3P)-3-(4-chloro-1-methyl-3-(methylsulfonamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydroquinazolin-7-yl)benzoic acid

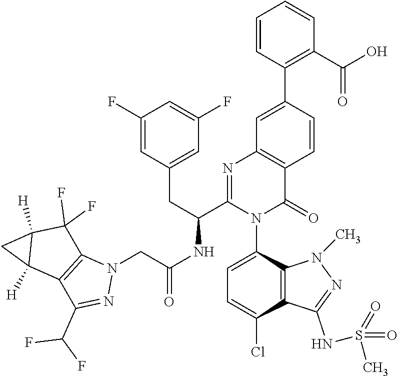

The title compound was prepared according to General Procedure K using 2-bromobenzoic acid as the coupling partner. The experiment afforded the title compound, 2-((3P)-3-(4-chloro-1-methyl-3-(methylsulfonamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydroquinazolin-7-yl)benzoic acid. The sample was analyzed using LCMS Method F: retention time=1.68 min.; observed ion=925.2 (M+H). 1H NMR (METHANOL-d4, 500 MHz) δ 8.3-8.3 (m, 1H), 8.00 (s, 1H), 7.7-7.8 (m, 2H), 7.5-7.6 (m, 2H), 7.31 (d, 1H, J=7.9 Hz), 7.1-7.2 (m, 1H), 6.7-6.8 (m, 1H), 6.62 (br d, 2H, J=6.7 Hz), 4.9-4.9 (m, 1H), 4.5-4.6 (m, 2H), 3.6-3.7 (m, 3H), 3.4-3.5 (m, 1H), 3.2-3.3 (m, 3H), 3.19 (br s, 1H), 3.10 (br dd, 1H, J=8.9, 13.7 Hz), 2.43 (br dd, 2H, J=3.5, 5.0 Hz), 1.3-1.4 (m, 2H), 1.01 (br s, 1H)

### Preparation of Example 5: N—((S)-1-((3P)-3-(4-chloro-1-methyl-3-(methylsulfonamido)-1H-indazol-7-yl)-7-(2-methoxypyrimidin-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

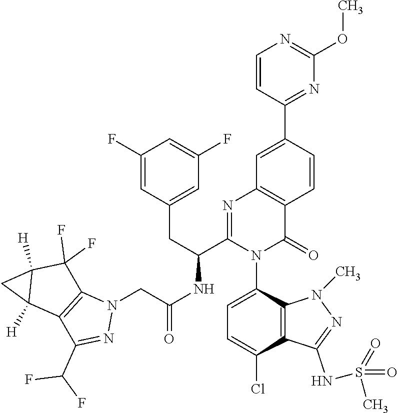

The title compound was prepared according to General Procedure L using 4-chloro-2-methoxypyrimidine as the coupling partner. The experiment afforded the title compound, N—((S)-1-((3P)-3-(4-chloro-1-methyl-3-(methylsulfonamido)-1H-indazol-7-yl)-7-(2-methoxypyrimidin-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.41 min.; observed ion=913.2 (M+H). 1H NMR (METHANOL-d4, 500 MHz) Shift 8.76 (d, 1H, J=5.1 Hz), 8.71 (d, 1H, J=1.5 Hz), 8.4-8.5 (m, 1H), 8.43 (d, 1H, J=1.5 Hz), 7.83 (d, 1H, J=5.1 Hz), 7.32 (d, 1H, J=7.7 Hz), 7.24 (d, 1H, J=7.7 Hz), 6.6-6.8 (m, 4H), 4.8-4.9 (m, 1H), 4.54 (d, 2H, J=3.3 Hz), 4.19 (s, 3H), 3.6-3.7 (m, 3H), 3.51 (dd, 1H, J=5.1, 14.0 Hz), 3.26 (s, 3H), 3.14 (dd, 1H, J=9.4, 13.9 Hz), 2.43 (br dd, 2H, J=4.6, 7.0 Hz), 1.36 (br d, 1H, J=5.7 Hz), 1.0-1.0 (m, 1H)

### Preparation of Example 6: N—((S)-1-((3P)-3-(4-chloro-1-methyl-3-(methylsulfonamido)-1H-indazol-7-yl)-7-(2,6-difluoropyridin-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

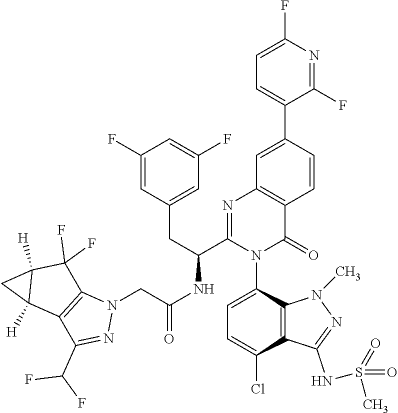

The title compound was prepared according to General Procedure L using 3-bromo-2,6-difluoropyridine as the coupling partner. The experiment afforded the title compound, N—((S)-1-((3P)-3-(4-chloro-1-methyl-3-(methylsulfonamido)-1H-indazol-7-yl)-7-(2,6-difluoropyridin-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.51 min.; observed ion=918.3 (M+H). 1H NMR (METHANOL-d4, 500 MHz) Shift 8.64 (d, 1H, J=2.4 Hz), 8.45 (t, 1H, J=1.3 Hz), 8.41 (d, 1H, J=8.9 Hz), 8.23 (td, 1H, J=1.5, 8.3 Hz), 7.84 (ddd, 1H, J=2.4, 8.4, 11.0 Hz), 7.32 (d, 1H, J=7.7 Hz), 7.24 (d, 1H, J=8.0 Hz), 6.6-6.8 (m, 4H), 4.8-4.9 (m, 1H), 4.54 (d, 2H, J=3.9 Hz), 3.64 (s, 3H), 3.50 (dd, 1H, J=4.8, 14.0 Hz), 3.25 (s, 3H), 3.13 (dd, 1H, J=9.2, 14.0 Hz), 2.43 (br dd, 2H, J=4.0, 6.1 Hz), 1.36 (br d, 1H, J=5.7 Hz), 1.01 (br dd, 1H, J=1.8, 3.6 Hz)

### Preparation of Example 7: N—((S)-1-((3P)-3-(4-chloro-1-methyl-3-(methylsulfonamido)-1H-indazol-7-yl)-7-(3,5-difluoropyridin-2-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

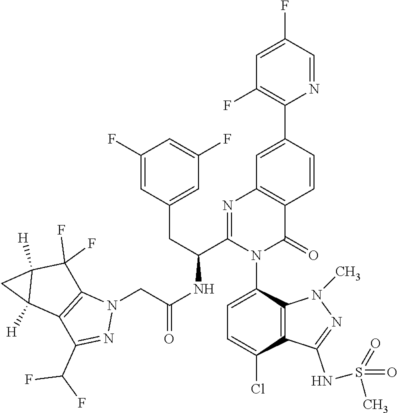

The title compound was prepared according to General Procedure L using 2-bromo-3,5-difluoropyridine as the coupling partner. The experiment afforded the title compound, N—((S)-1-((3P)-3-(4-chloro-1-methyl-3-(methylsulfonamido)-1H-indazol-7-yl)-7-(3,5-difluoropyridin-2-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.49 min.; observed ion=918.3 (M+H). 1H NMR (METHANOL-d4, 500 MHz) Shift 8.42 (s, 1H), 8.40 (s, 1H), 8.13 (s, 1H), 7.88 (td, 1H, J=1.4, 8.3 Hz), 7.32 (d, 1H, J=8.0 Hz), 7.24 (s, 1H), 7.2-7.2 (m, 1H), 6.8-6.8 (m, 1H), 6.63 (dd, 2H, J=2.2, 8.2 Hz), 6.69 (t, 1H, J=54.7 Hz), 4.62 (br s, 2H), 4.53 (d, 2H, J=2.7 Hz), 3.63 (s, 3H), 3.50 (dd, 1H, J=5.2, 14.2 Hz), 3.2-3.3 (m, 3H), 3.12 (dd, 1H, J=9.2, 14.0 Hz), 2.43 (dt, 2H, J=4.0, 7.5 Hz), 1.3-1.4 (m, 1H), 1.0-1.0 (m, 1H)

### Preparation of Example 8: N—((S)-1-((3P)-3-(4-chloro-1-methyl-3-(methylsulfonamido)-1H-indazol-7-yl)-7-(5-chloro-3-fluoropyridin-2-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

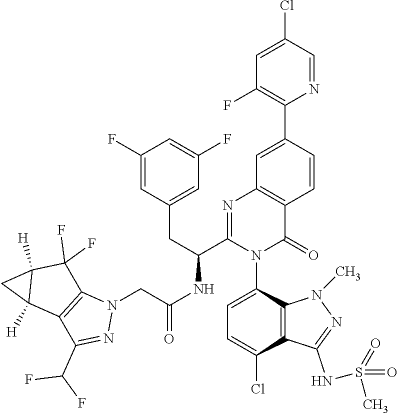

The title compound was prepared according to General Procedure L using 2-bromo-5-chloro-3-fluoropyridine as the coupling partner. The experiment afforded the title compound, N—((S)-1-((3P)-3-(4-chloro-1-methyl-3-(methylsulfonamido)-1H-indazol-7-yl)-7-(5-chloro-3-fluoropyridin-2-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.58 min.; observed ion=932.1 (M−H). 1H NMR (METHANOL-d4, 500 MHz) Shift 8.68 (dd, 1H, J=1.0, 1.9 Hz), 8.48 (t, 1H, J=1.3 Hz), 8.41 (d, 1H, J=8.2 Hz), 8.26 (td, 1H, J=1.5, 8.3 Hz), 8.03 (dd, 1H, J=1.9, 10.9 Hz), 7.32 (d, 1H, J=8.0 Hz), 7.24 (d, 1H, J=7.7 Hz), 6.6-6.8 (m, 4H), 4.8-4.8 (m, 1H), 4.54 (d, 2H, J=4.2 Hz), 3.64 (s, 3H), 3.50 (dd, 1H, J=5.1, 14.0 Hz), 3.2-3.3 (m, 3H), 3.12 (dd, 1H, J=9.2, 14.0 Hz), 2.4-2.5 (m, 2H), 1.3-1.4 (m, 1H), 1.0-1.0 (m, 1H)

### Preparation of Example 9: N—((S)-1-((3P)-3-(4-chloro-1-methyl-3-(methylsulfonamido)-1H-indazol-7-yl)-7-(2-(methylsulfonyl)pyrimidin-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

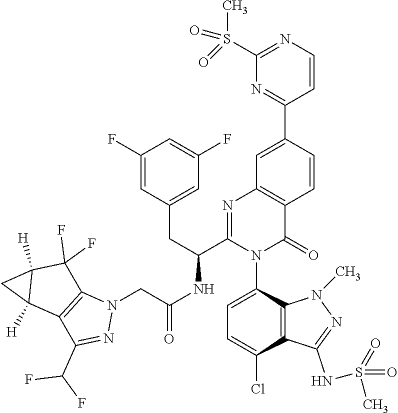

The title compound was prepared according to General Procedure L using 4-chloro-2-(methylsulfonyl)pyrimidine as the coupling partner. The experiment afforded the title compound, N—((S)-1-((3P)-3-(4-chloro-1-methyl-3-(methylsulfonamido)-1H-indazol-7-yl)-7-(2-(methylsulfonyl)pyrimidin-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.33 min.; observed ion=961.2 (M+H). 1H NMR (METHANOL-d4, 500 MHz) Shift 9.18 (d, 1H, J=5.4 Hz), 8.83 (d, 1H, J=0.9 Hz), 8.57 (s, 1H), 8.5-8.5 (m, 3H), 7.32 (d, 1H, J=7.7 Hz), 7.25 (d, 1H, J=8.0 Hz), 6.6-6.8 (m, 4H), 4.54 (d, 2H, J=3.3 Hz), 3.64 (s, 3H), 3.5-3.5 (m, 4H), 3.26 (s, 3H), 3.14 (dd, 1H, J=9.2, 14.0 Hz), 2.4-2.5 (m, 2H), 1.37 (br d, 1H, J=6.0 Hz), 1.00 (br dd, 1H, J=1.8, 3.6 Hz)

### Preparation of Example 10: N—((S)-1-((3P)-3-(4-chloro-1-methyl-3-(methylsulfonamido)-1H-indazol-7-yl)-7-(3-chloro-5-fluoropyridin-2-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

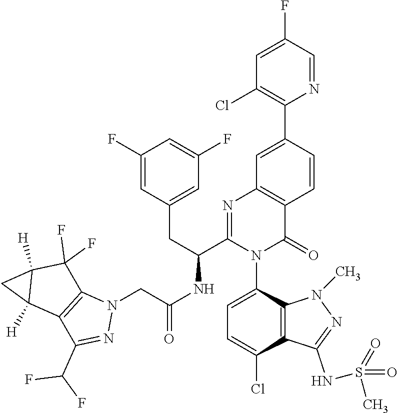

The title compound was prepared according to General Procedure L using 2-bromo-3-chloro-5-fluoropyridine as the coupling partner. The experiment afforded the title compound, N—((S)-1-((3P)-3-(4-chloro-1-methyl-3-(methylsulfonamido)-1H-indazol-7-yl)-7-(3-chloro-5-fluoropyridin-2-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.52 min.; observed ion=934.3 (M−H). 1H NMR (METHANOL-d4, 500 MHz) Shift 8.67 (d, 1H, J=2.7 Hz), 8.40 (d, 1H, J=8.0 Hz), 8.20 (s, 1H), 8.0-8.1 (m, 1H), 7.95 (dd, 1H, J=1.8, 8.3 Hz), 7.32 (d, 1H, J=8.0 Hz), 7.24 (d, 1H, J=7.7 Hz), 6.79 (br t, 1H, J=2.4 Hz), 6.63 (dd, 2H, J=2.2, 8.2 Hz), 6.69 (br t, 1H, J=54.7 Hz), 4.8-4.9 (m, 1H), 4.53 (d, 2H, J=3.6 Hz), 3.65 (s, 3H), 3.4-3.5 (m, 1H), 3.2-3.3 (m, 3H), 3.11 (dd, 1H, J=9.2, 14.0 Hz), 2.43 (br dd, 2H, J=3.3, 6.9 Hz), 1.36 (s, 1H), 1.0-1.0 (m, 1H)

### Preparation of Example 11: N—((S)-1-((3P)-3-(4-chloro-1-methyl-3-(methylsulfonamido)-1H-indazol-7-yl)-7-(2-cyanopyrimidin-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

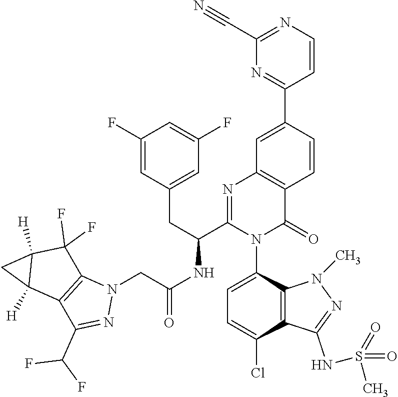

The title compound was prepared according to General Procedure L using 4-chloropyrimidine-2-carbonitrile as the coupling partner. The experiment afforded the title compound, N—((S)-1-((3P)-3-(4-chloro-1-methyl-3-(methylsulfonamido)-1H-indazol-7-yl)-7-(2-cyanopyrimidin-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.44 min.; observed ion=906.3 (M−H). 1H NMR (METHANOL-d4, 500 MHz) Shift 9.10 (d, 1H, J=5.4 Hz), 8.76 (s, 1H), 8.4-8.5 (m, 3H), 7.32 (d, 1H, J=7.7 Hz), 7.25 (d, 1H, J=7.7 Hz), 6.8-6.8 (m, 1H), 6.64 (dd, 2H, J=2.2, 8.2 Hz), 6.70 (br t, 1H, J=54.7 Hz), 4.8-4.9 (m, 1H), 4.54 (d, 2H, J=3.9 Hz), 3.64 (s, 3H), 3.5-3.5 (m, 1H), 3.25 (s, 3H), 3.14 (dd, 1H, J=9.4, 14.2 Hz), 2.4-2.5 (m, 2H), 1.37 (s, 1H), 1.0-1.0 (m, 1H)

### Preparation of Example 12: N—((S)-1-((3P)-3-(4-chloro-1-methyl-3-(methylsulfonamido)-1H-indazol-7-yl)-7-(4-cyano-6-methylpyrimidin-2-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

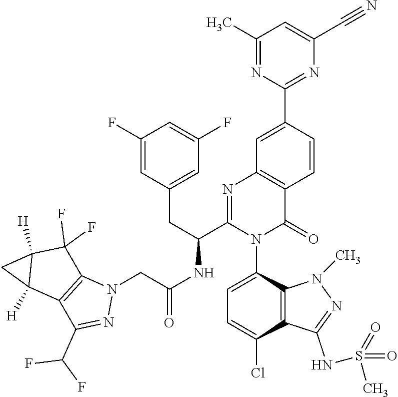

The title compound was prepared according to General Procedure L using 2-chloro-6-methylpyrimidine-4-carbonitrile as the coupling partner. The experiment afforded the title compound, N—((S)-1-((3P)-3-(4-chloro-1-methyl-3-(methylsulfonamido)-1H-indazol-7-yl)-7-(4-cyano-6-methylpyrimidin-2-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.51 min.; observed ion=920.4 (M−H). 1H NMR (METHANOL-d4, 500 MHz) Shift 9.0-9.0 (m, 1H), 8.7-8.7 (m, 1H), 8.43 (d, 1H, J=8.8 Hz), 7.86 (s, 1H), 7.33 (d, 1H, J=7.7 Hz), 7.26 (d, 1H, J=8.0 Hz), 6.6-6.8 (m, 4H), 4.8-4.9 (m, 1H), 4.5-4.6 (m, 2H), 3.64 (s, 3H), 3.51 (dd, 1H, J=4.8, 14.0 Hz), 3.26 (s, 3H), 3.13 (dd, 1H, J=9.2, 14.0 Hz), 2.79 (s, 3H), 2.44 (br d, 2H, J=12.5 Hz), 1.37 (br dd, 1H, J=1.5, 6.9 Hz), 1.02 (td, 1H, J=2.0, 3.7 Hz)

### Preparation of Example 16: N—((S)-1-((3P)-3-(4-chloro-1-methyl-3-(methylsulfonamido)-1H-indazol-7-yl)-7-(2-methoxy-6-methylpyrimidin-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

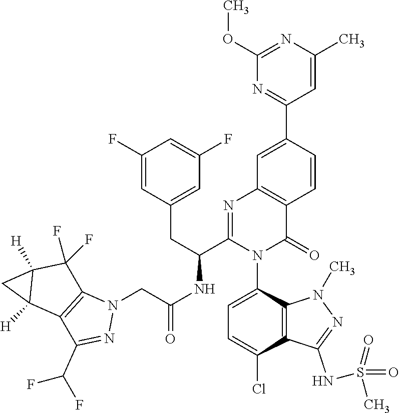

The title compound was prepared according to General Procedure L using 4-chloro-2-methoxy-6-methylpyrimidine as the coupling partner. The experiment afforded the title compound, N—((S)-1-((3P)-3-(4-chloro-1-methyl-3-(methylsulfonamido)-1H-indazol-7-yl)-7-(2-methoxy-6-methylpyrimidin-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.46 min.; observed ion=927.4 (M+H). 1H NMR (METHANOL-d4, 500 MHz) Shift 8.70 (s, 1H), 8.4-8.4 (m, 2H), 7.74 (s, 1H), 7.32 (d, 1H, J=7.7 Hz), 7.24 (d, 1H, J=7.7 Hz), 6.80 (br t, 1H, J=2.4 Hz), 6.70 (br t, 1H, J=54.8 Hz), 6.64 (dd, 2H, J=2.4, 8.0 Hz), 4.8-4.9 (m, 1H), 4.54 (d, 2H, J=3.3 Hz), 4.18 (s, 3H), 3.63 (s, 3H), 3.51 (dd, 1H, J=4.8, 14.0 Hz), 3.2-3.3 (m, 3H), 3.13 (dd, 1H, J=9.4, 14.2 Hz), 2.62 (s, 3H), 2.43 (br dd, 2H, J=4.0, 6.4 Hz), 1.36 (s, 1H), 1.0-1.0 (m, 1H)

### Preparation of Example 17: N—((S)-1-((3P)-3-(4-chloro-1-methyl-3-(methylsulfonamido)-1H-indazol-7-yl)-7-(5-(difluoromethyl)pyridin-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

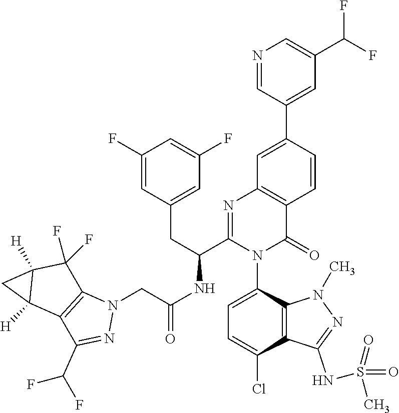

The title compound was prepared according to General Procedure L using 3-bromo-5-(difluoromethyl)pyridine as the coupling partner. The experiment afforded the title compound, N—((S)-1-((3P)-3-(4-chloro-1-methyl-3-(methylsulfonamido)-1H-indazol-7-yl)-7-(5-(difluoromethyl)pyridin-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.52 min.; observed ion=932.1 (M+H). 1H NMR (METHANOL-d4, 500 MHz) Shift 8.6-8.7 (m, 1H), 8.4-8.4 (m, 2H), 8.28 (d, 1H, J=7.8 Hz), 8.18 (t, 1H, J=7.9 Hz), 7.79 (d, 1H, J=7.7 Hz), 7.32 (d, 1H, J=8.0 Hz), 7.23 (d, 1H, J=7.7 Hz), 6.6-7.0 (m, 5H), 4.8-4.9 (m, 1H), 4.54 (d, 2H, J=3.9 Hz), 3.64 (s, 3H), 3.51 (dd, 1H, J=5.1, 14.0 Hz), 3.26 (s, 3H), 3.14 (dd, 1H, J=9.2, 14.0 Hz), 2.44 (br dd, 2H, J=4.0, 7.6 Hz), 1.36 (s, 1H), 1.01 (br dd, 1H, J=1.9, 3.4 Hz)

### Preparation of Example 18: N—((S)-1-((3P)-3-(4-chloro-1-methyl-3-(methylsulfonamido)-1H-indazol-7-yl)-7-(3-(difluoromethyl)-2-fluorophenyl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

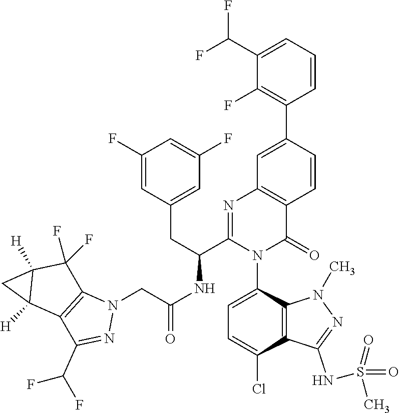

The title compound was prepared according to General Procedure L using 1-bromo-3-(difluoromethyl)-2-fluorobenzene as the coupling partner. The experiment afforded the title compound, N—((S)-1-((3P)-3-(4-chloro-1-methyl-3-(methylsulfonamido)-1H-indazol-7-yl)-7-(3-(difluoromethyl)-2-fluorophenyl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.55 min.; observed ion=947.2 (M−H). 1H NMR (METHANOL-d4, 500 MHz) Shift 8.40 (d, 1H, J=8.1 Hz), 8.10 (t, 1H, J=1.5 Hz), 7.8-7.9 (m, 2H), 7.77 (t, 1H, 3=6.9 Hz), 7.53 (t, 1H, 3=7.7 Hz), 7.32 (d, 1H, 3=7.7 Hz), 7.24 (d, 1H, 3=7.7 Hz), 7.15 (br t, 1H, J=54.7 Hz), 6.6-6.8 (m, 4H), 4.8-4.8 (m, 1H), 4.54 (d, 2H, J=4.5 Hz), 3.64 (s, 3H), 3.5-3.5 (m, 1H), 3.2-3.3 (m, 3H), 3.12 (dd, 1H, J=9.2, 14.0 Hz), 2.43 (dt, 2H, J=4.0, 7.7 Hz), 1.36 (br d, 1H, J=7.7 Hz), 1.01 (br dd, 1H, J=1.8, 3.6 Hz)

### Preparation of Example 19: N—((S)-1-((3P)-3-(4-chloro-1-methyl-3-(methylsulfonamido)-1H-indazol-7-yl)-7-(2-(methoxymethyl)phenyl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

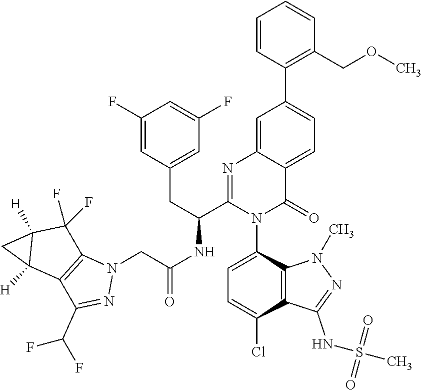

The title compound was prepared according to General Procedure K using (2-(methoxymethyl)phenyl)boronic acid as the coupling partner. The experiment afforded the title compound, N—((S)-1-((3P)-3-(4-chloro-1-methyl-3-(methylsulfonamido)-1H-indazol-7-yl)-7-(2-(methoxymethyl)phenyl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.47 min.; observed ion=925.8 (M+H). 1H NMR (500 MHz, METHANOL-d4) δ ppm 8.35 (d, J=8.64 Hz, 1H), 7.92 (d, J=1.19 Hz, 1H), 7.66-7.73 (m, 1H), 7.58-7.64 (m, 1H), 7.49-7.53 (m, 2H), 7.42-7.46 (m, 1H), 7.32 (d, J=7.75 Hz, 1H), 7.22 (d, J=8.05 Hz, 1H), 6.54-6.84 (m, 4H), 4.87-4.90 (m, 1H), 4.54 (d, J=2.38 Hz, 2H), 4.44 (s, 2H), 3.65 (s, 3H), 3.47-3.52 (m, 1H), 3.36 (s, 3H), 3.26 (s, 3H), 3.11 (dd, J=14.01, 8.94 Hz, 1H), 2.40-2.47 (m, 2H), 1.35-1.39 (m, 1H), 0.97-1.06 (m, 1H).

### Preparation of Example 20: N—((S)-1-((3P)-3-(4-chloro-1-methyl-3-(methylsulfonamido)-1H-indazol-7-yl)-7-(1-methyl-1H-pyrazol-5-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

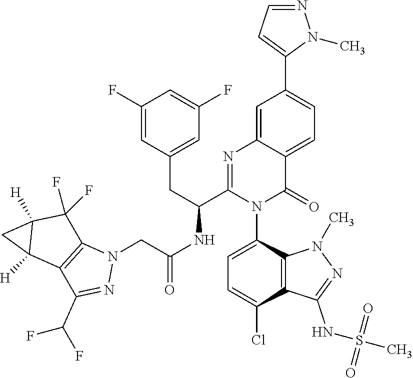

The title compound was prepared according to General Procedure K using (1-methyl-1H-pyrazol-5-yl)boronic acid as the coupling partner. The experiment afforded the title compound, N—((S)-1-((3P)-3-(4-chloro-1-methyl-3-(methylsulfonamido)-1H-indazol-7-yl)-7-(1-methyl-1H-pyrazol-5-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.29 min.; observed ion=885.2 (M+H). 1H NMR (500 MHz, METHANOL-d4) δ ppm 8.41 (d, J=7.75 Hz, 1H), 8.02 (d, J=1.19 Hz, 1H), 7.82 (dd, J=8.20, 1.64 Hz, 1H), 7.63 (d, J=2.09 Hz, 1H), 7.32 (d, J=8.05 Hz, 1H), 7.23 (d, J=7.75 Hz, 1H), 6.57-6.82 (m, 5H), 4.87-4.88 (m, 1H), 4.54 (d, J=4.17 Hz, 2H), 4.05 (s, 3H), 3.64 (s, 3H), 3.47-3.51 (m, 1H), 3.26 (s, 3H), 3.09-3.15 (m, 1H), 2.39-2.47 (m, 2H), 1.34-1.39 (m, 1H), 0.98-1.03 (m, 1H).

### Preparation of Example 21: N—((S)-1-((3P)-3-(4-chloro-1-methyl-3-(methylsulfonamido)-1H-indazol-7-yl)-7-(3-fluoro-6-(trifluoromethyl)pyridin-2-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

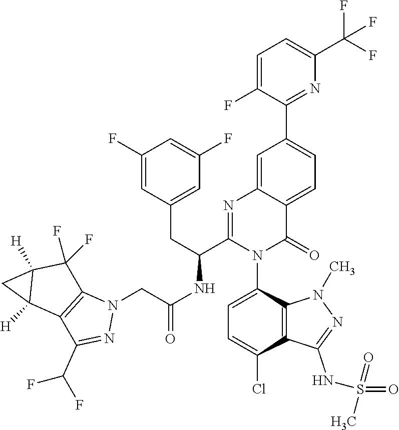

The title compound was prepared according to General Procedure L using 2-chloro-3-fluoro-6-(trifluoromethyl)pyridine as the coupling partner. The experiment afforded the title compound, N—((S)-1-((3P)-3-(4-chloro-1-methyl-3-(methylsulfonamido)-1H-indazol-7-yl)-7-(3-fluoro-6-(trifluoromethyl)pyridin-2-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.6 min.; observed ion=968.2 (M+H). 1H NMR (500 MHz, METHANOL-d4) δ ppm 8.57 (s, 1H), 8.45 (d, J=8.35 Hz, 1H), 8.29-8.35 (m, 1H), 8.04-8.09 (m, 1H), 7.99-8.02 (m, 1H), 7.31-7.36 (m, 1H), 7.26 (d, J=7.75 Hz, 1H), 6.55-6.84 (m, 4H), 4.90-4.92 (m, 1H), 4.49-4.62 (m, 2H), 3.65 (s, 3H), 3.45-3.53 (m, 1H), 3.26 (s, 3H), 3.14 (dd, J=14.16, 9.39 Hz, 1H), 2.39-2.49 (m, 2H), 1.35-1.38 (m, 1H), 0.97-1.04 (m, 1H).

### Preparation of Example 22: N—((S)-1-((3P)-3-(4-chloro-1-methyl-3-(methylsulfonamido)-1H-indazol-7-yl)-4-oxo-7-(6-(trifluoromethyl)pyrazin-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

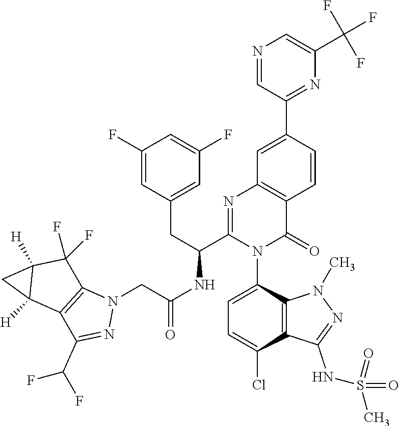

The title compound was prepared according to General Procedure L using 2-chloro-6-(trifluoromethyl)pyrazine as the coupling partner. The experiment afforded the title compound, N—((S)-1-((3P)-3-(4-chloro-1-methyl-3-(methylsulfonamido)-1H-indazol-7-yl)-4-oxo-7-(6-(trifluoromethyl)pyrazin-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.53 min.; observed ion=951.2 (M+H). 1H NMR (500 MHz, METHANOL-d4) δ ppm 9.67 (s, 1H), 9.15 (s, 1H), 8.66-8.77 (m, 1H), 8.43-8.53 (m, 2H), 7.33 (d, J=8.05 Hz, 1H), 7.25 (d, J=7.75 Hz, 1H), 6.53-6.87 (m, 4H), 4.89-4.93 (m, 1H), 4.49-4.60 (m, 2H), 3.64 (s, 3H), 3.51 (dd, J=14.01, 5.07 Hz, 1H), 3.26 (s, 3H), 3.14 (dd, J=14.16, 9.39 Hz, 1H), 2.39-2.52 (m, 2H), 1.33-1.40 (m, 1H), 0.97-1.06 (m, 1H).

### Preparation of Example 23: N—((S)-1-((3P)-3-(4-chloro-1-methyl-3-(methylsulfonamido)-1H-indazol-7-yl)-4-oxo-7-(3-(trifluoromethyl)pyrazin-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

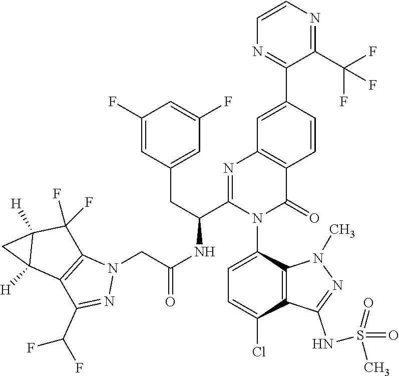

The title compound was prepared according to General Procedure L using 2-chloro-3-(trifluoromethyl)pyrazine as the coupling partner. The experiment afforded the title compound, N—((S)-1-((3P)-3-(4-chloro-1-methyl-3-(methylsulfonamido)-1H-indazol-7-yl)-4-oxo-7-(3-(trifluoromethyl)pyrazin-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.46 min.; observed ion=951.2 (M+H). 1H NMR (500 MHz, METHANOL-d4) δ ppm 9.04 (d, J=2.38 Hz, 1H), 8.89 (d, J=2.38 Hz, 1H), 8.44 (d, J=8.34 Hz, 1H), 8.07 (d, J=1.49 Hz, 1H), 7.85 (dd, J=8.20, 1.64 Hz, 1H), 7.30-7.36 (m, 1H), 7.24-7.30 (m, 1H), 6.54-6.83 (m, 4H), 4.89-4.92 (m, 1H), 4.47-4.58 (m, 2H), 3.67 (s, 3H), 3.46-3.53 (m, 1H), 3.26 (s, 3H), 3.12 (dd, J=14.16, 9.39 Hz, 1H), 2.39-2.48 (m, 2H), 1.34-1.40 (m, 1H), 0.98-1.04 (m, 1H).

### Preparation of Example 24: N—((S)-1-((3P)-3-(4-chloro-1-methyl-3-(methylsulfonamido)-1H-indazol-7-yl)-7-(5-fluoropyrimidin-2-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

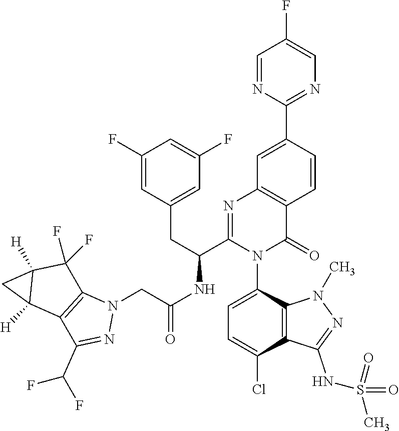

The title compound was prepared according to General Procedure L using 2-chloro-5-fluoropyrimidine as the coupling partner. The experiment afforded the title compound, N—((S)-1-((3P)-3-(4-chloro-1-methyl-3-(methylsulfonamido)-1H-indazol-7-yl)-7-(5-fluoropyrimidin-2-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.48 min.; observed ion=901.3 (M+H). 1H NMR (500 MHz, METHANOL-d4) δ ppm 8.95 (s, 2H), 8.92 (d, J=1.49 Hz, 1H), 8.68 (dd, J=8.49, 1.64 Hz, 1H), 8.41 (d, J=8.34 Hz, 1H), 7.32 (d, J=7.75 Hz, 1H), 7.23 (d, J=7.75 Hz, 1H), 6.53-6.84 (m, 4H), 4.89-4.92 (m, 1H), 4.51-4.59 (m, 2H), 3.64 (s, 3H), 3.48-3.54 (m, 1H), 3.26 (s, 3H), 3.13 (dd, J=14.16, 9.09 Hz, 1H), 2.40-2.50 (m, 2H), 1.34-1.39 (m, 1H), 0.96-1.05 (m, 1H)

### Preparation of Example 25: N—((S)-1-((3P)-3-(4-chloro-1-methyl-3-(methylsulfonamido)-1H-indazol-7-yl)-4-oxo-7-(6-(trifluoromethyl)pyrimidin-4-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

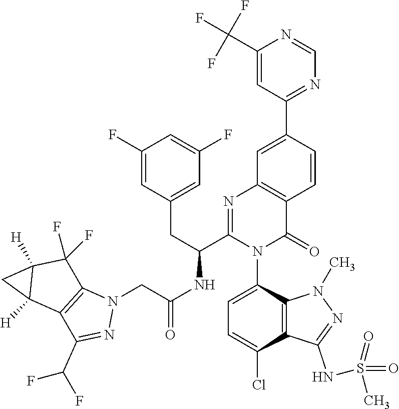

The title compound was prepared according to General Procedure L using 4-chloro-6-(trifluoromethyl)pyrimidine as the coupling partner. The experiment afforded the title compound, N—((S)-1-((3P)-3-(4-chloro-1-methyl-3-(methylsulfonamido)-1H-indazol-7-yl)-4-oxo-7-(6-(trifluoromethyl)pyrimidin-4-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.53 min.; observed ion=951.3 (M+H). 1H NMR (500 MHz, METHANOL-d4) δ ppm 8.35-9.00 (m, 3H), 7.52-7.81 (m, 1H), 7.29-7.35 (m, 1H), 7.21-7.26 (m, 1H), 6.50-6.85 (m, 4H), 4.90-4.92 (m, 1H), 4.50-4.55 (m, 2H), 3.62-3.68 (m, 3H), 3.46-3.56 (m, 1H), 3.26 (s, 3H), 3.09-3.16 (m, 1H), 2.38-2.49 (m, 2H), 1.33-1.42 (m, 1H), 0.99-1.06 (m, 1H).

### Preparation of Example 26: N—((S)-1-((3P)-3-(4-chloro-1-methyl-3-(methylsulfonamido)-1H-indazol-7-yl)-7-(6-methyl-2-(trifluoromethyl)pyrimidin-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

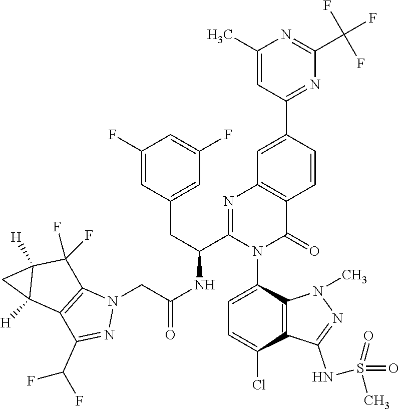

The title compound was prepared according to General Procedure L using 4-chloro-6-methyl-2-(trifluoromethyl)pyrimidine as the coupling partner. The experiment afforded the title compound, N—((S)-1-((3P)-3-(4-chloro-1-methyl-3-(methylsulfonamido)-1H-indazol-7-yl)-7-(6-methyl-2-(trifluoromethyl)pyrimidin-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.56 min.; observed ion=965.2 (M+H). 1H NMR (500 MHz, METHANOL-d4) δ ppm 8.79 (t, J=1.04 Hz, 1H), 8.47 (d, J=1.19 Hz, 2H), 8.34 (s, 1H), 7.30-7.36 (m, 1H), 7.24 (d, J=7.75 Hz, 1H), 6.53-6.85 (m, 4H), 4.89-4.92 (m, 1H), 4.49-4.59 (m, 2H), 3.64 (s, 3H), 3.51 (dd, J=14.01, 5.07 Hz, 1H), 3.26 (s, 3H), 3.14 (dd, J=14.16, 9.39 Hz, 1H), 2.79 (s, 3H), 2.37-2.49 (m, 2H), 1.34-1.39 (m, 1H), 0.98-1.03 (m, 1H).

### Preparation of Example 27: N—((S)-1-((3P)-3-(4-chloro-1-methyl-3-(methylsulfonamido)-1H-indazol-7-yl)-4-oxo-7-(2-(trifluoromethyl)pyrimidin-4-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

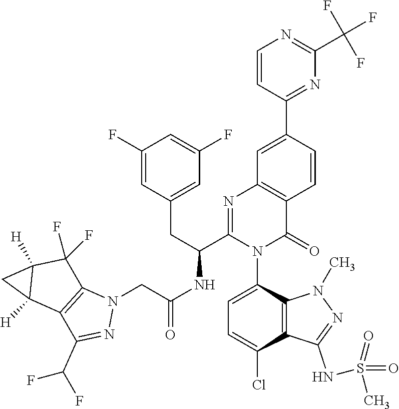

The title compound was prepared according to General Procedure L using 4-chloro-2-(trifluoromethyl)pyrimidine as the coupling partner. The experiment afforded the title compound, N—((S)-1-((3P)-3-(4-chloro-1-methyl-3-(methylsulfonamido)-1H-indazol-7-yl)-4-oxo-7-(2-(trifluoromethyl)pyrimidin-4-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.52 min.; observed ion=951.2 (M+H). 1H NMR (500 MHz, METHANOL-d4) δ ppm 9.16 (d, J=5.07 Hz, 1H), 8.81 (t, J=1.19 Hz, 1H), 8.49 (d, J=0.89 Hz, 2H), 8.44 (d, J=5.36 Hz, 1H), 7.33 (d, J=7.75 Hz, 1H), 7.25 (d, J=7.75 Hz, 1H), 6.56-6.84 (m, 4H), 4.89-4.92 (m, 1H), 4.49-4.59 (m, 2H), 3.64 (s, 3H), 3.51 (dd, J=14.16, 4.92 Hz, 1H), 3.26 (s, 3H), 3.14 (dd, J=14.16, 9.39 Hz, 1H), 2.39-2.48 (m, 2H), 1.34-1.41 (m, 1H), 0.98-1.04 (m, 1H).

### Preparation of Example 28: N—((S)-1-((3P)-3-(4-chloro-1-methyl-3-(methylsulfonamido)-1H-indazol-7-yl)-7-(6-(1,1-difluoroethyl)pyridin-2-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

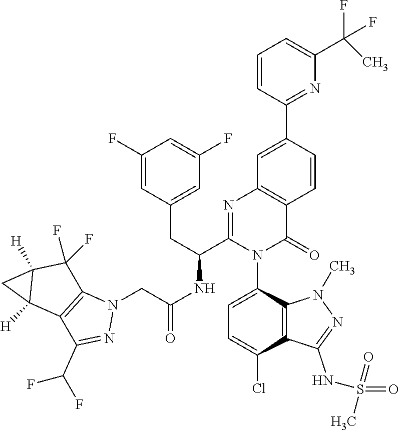

The title compound was prepared according to General Procedure L using 2-chloro-6-(1,1-difluoroethyl)pyridine as the coupling partner. The experiment afforded the title compound, N—((S)-1-((3P)-3-(4-chloro-1-methyl-3-(methylsulfonamido)-1H-indazol-7-yl)-7-(6-(1,1-difluoroethyl)pyridin-2-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.6 min.; observed ion=946.2 (M+H). 1H NMR (500 MHz, METHANOL-d4) δ ppm 8.63-8.70 (m, 1H), 8.36-8.45 (m, 2H), 8.24 (d, J=7.75 Hz, 1H), 8.14 (t, J=7.90 Hz, 1H), 7.80 (dd, J=7.90, 0.75 Hz, 1H), 7.32 (d, J=7.75 Hz, 1H), 7.24 (d, J=7.75 Hz, 1H), 6.55-6.85 (m, 4H), 4.89-4.92 (m, 1H), 4.49-4.63 (m, 2H), 3.64 (s, 3H), 3.51 (dd, J=14.01, 5.07 Hz, 1H), 3.26 (s, 3H), 3.14 (dd, J=14.31, 9.24 Hz, 1H), 2.38-2.48 (m, 2H), 2.16 (t, J=18.78 Hz, 3H), 1.34-1.39 (m, 1H), 1.01 (dtd, J=5.70, 3.78, 3.78, 2.53 Hz, 1H).

### Preparation of Example 29: N—((S)-1-((3P)-3-(4-chloro-1-methyl-3-(methylsulfonamido)-1H-indazol-7-yl)-7-(1-neopentyl-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

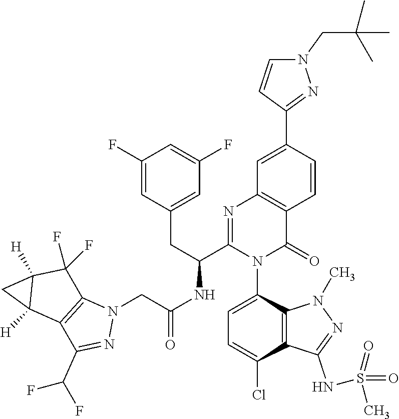

The title compound was prepared according to General Procedure L using 3-bromo-1-neopentyl-1H-pyrazole as the coupling partner. The experiment afforded the title compound, N—((S)-1-((3P)-3-(4-chloro-1-methyl-3-(methylsulfonamido)-1H-indazol-7-yl)-7-(1-neopentyl-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.6 min.; observed ion=941.4 (M+H). 1H NMR (500 MHz, METHANOL-d4) δ ppm 10.79-10.80 (m, 1H), 8.26-8.34 (m, 2H), 8.11 (dd, J=8.34, 1.49 Hz, 1H), 7.74 (d, J=2.38 Hz, 1H), 7.30 (d, J=8.05 Hz, 1H), 7.19 (d, J=8.05 Hz, 1H), 6.89 (d, J=2.38 Hz, 1H), 6.56-6.83 (m, 4H), 4.89-4.92 (m, 1H), 4.55 (d, J=2.98 Hz, 2H), 4.09 (s, 2H), 3.63 (s, 3H), 3.49 (dd, J=14.31, 5.36 Hz, 1H), 3.25 (s, 3H), 3.11 (dd, J=14.01, 9.24 Hz, 1H), 2.39-2.49 (m, 2H), 1.33-1.40 (m, 1H), 1.06 (s, 9H), 0.99-1.03 (m, 1H).

### Preparation of Example 30: N—((S)-1-((3P)-3-(4-chloro-1-methyl-3-(methylsulfonamido)-1H-indazol-7-yl)-7-(5-fluoro-6-(trifluoromethyl)pyridin-2-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

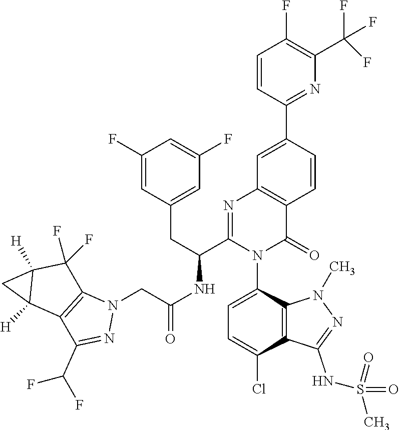

The title compound was prepared according to General Procedure L using 6-chloro-3-fluoro-2-(trifluoromethyl)pyridine as the coupling partner. The experiment afforded the title compound, N—((S)-1-((3P)-3-(4-chloro-1-methyl-3-(methylsulfonamido)-1H-indazol-7-yl)-7-(5-fluoro-6-(trifluoromethyl)pyridin-2-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.61 min.; observed ion=968.1 (M+H). 1H NMR (500 MHz, METHANOL-d4) δ ppm 8.64 (d, J=1.19 Hz, 1H), 8.50 (dd, J=8.64, 3.28 Hz, 1H), 8.41-8.45 (m, 1H), 8.34-8.39 (m, 1H), 8.07 (t, J=9.39 Hz, 1H), 7.31 (d, J=8.05 Hz, 1H), 7.22 (d, J=7.75 Hz, 1H), 6.61-6.84 (m, 4H), 4.89-4.91 (m, 1H), 4.55 (d, J=5.36 Hz, 2H), 3.63 (s, 3H), 3.50 (dd, J=14.16, 4.92 Hz, 1H), 3.25 (s, 3H), 3.14 (dd, J=14.16, 9.39 Hz, 1H), 2.38-2.49 (m, 2H), 1.33-1.39 (m, 1H), 0.97-1.04 (m, 1H).

### Preparation of Example 31: N—((S)-1-((3P)-3-(4-chloro-1-methyl-3-(methylsulfonamido)-1H-indazol-7-yl)-7-(1-(cyclopropylmethyl)-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

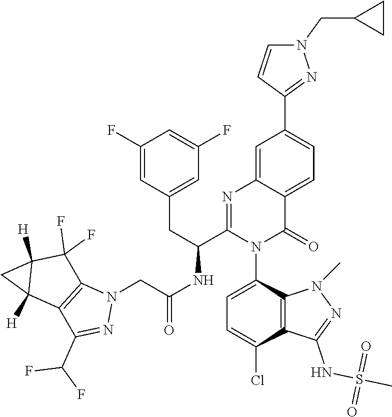

A solution of N—((S)-1-((3P)-3-(4-chloro-3-(N-(4-methoxybenzyl)methylsulfonamido)-1-methyl-1H-indazol-7-yl)-4-oxo-7-(tributylstannyl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (65 mg, 0.054 mmol) and 3-bromo-1-(cyclopropylmethyl)-1H-pyrazole (21.52 mg, 0.107 mmol) in Toluene (1 mL) was purged with argon and then to the solution was added bis(triphenylphosphine)palladium(II) chloride (3.76 mg, 5.35 μmol). The mixture was stirred at 100° C. for 16 h. The mixture was then cooled to room temperature and then was concentrated in vacuo. The residue was dissolved in DCM (0.5 mL) and TFA (1 mL) and to the solution was added triflic acid (0.05 mL). The solution was stirred at rt for 15 min. and then was concentrated in vacuo. The residue dissolved in DMF, filtered, and subjected to HPLC purification to afford the title compound, N—((S)-1-((3P)-3-(4-chloro-1-methyl-3-(methylsulfonamido)-1H-indazol-7-yl)-7-(1-(cyclopropylmethyl)-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.5 min.; observed ion=925.3 (M+H). 1H NMR (500 MHz, METHANOL-d4) δ ppm 8.28-8.33 (m, 2H), 8.11 (dd, J=8.34, 1.79 Hz, 1H), 7.85 (d, J=2.38 Hz, 1H), 7.31 (d, J=7.75 Hz, 1H), 7.20 (d, J=7.75 Hz, 1H), 6.90 (d, J=2.38 Hz, 1H), 6.58-6.83 (m, 4H), 4.54 (d, J=1.49 Hz, 2H), 4.15 (d, J=6.85 Hz, 2H), 3.64 (s, 3H), 3.46-3.53 (m, 1H), 3.34-3.38 (m, 1H), 3.26 (s, 3H), 3.11 (dd, J=14.01, 9.24 Hz, 1H), 2.40-2.52 (m, 2H), 1.34-1.46 (m, 2H), 0.98-1.04 (m, 1H), 0.66-0.75 (m, 2H), 0.48-0.54 (m, 2H).

### Preparation of Example 32: N—((S)-1-((3P)-3-(4-chloro-1-methyl-3-(methylsulfonamido)-1H-indazol-7-yl)-4-oxo-7-(5-(trifluoromethyl)-1H-pyrazol-1-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

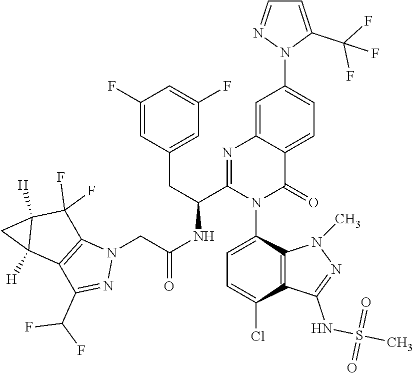

To a 5 mL microwave charged with N—((S)-1-((3P)-3-(4-chloro-1-methyl-3-(methylsulfonamido)-1H-indazol-7-yl)-4-oxo-7-(4,4,5,5-tetramethyl-1,3,2-dioxaborolan-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (50 mg, 0.054 mmol), 5-(trifluoromethyl)-1H-pyrazole (8.77 mg, 0.064 mmol), copper (II) acetate (2.93 mg, 0.016 mmol), boric acid (6.64 mg, 0.107 mmol) and powdered activated 4 A molecular sieves (25 mg) was added acetonitrile (2 mL). The mixture was stirred under air at 80° C. for 16 h. The mixture was cooled to room temperature, filtered and the filtrated was concentrated in vacuo. The residue was subjected to HPLC purification to afford the title compound, N—((S)-1-((3P)-3-(4-chloro-1-methyl-3-(methylsulfonamido)-1H-indazol-7-yl)-4-oxo-7-(5-(trifluoromethyl)-1H-pyrazol-1-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.55 min.; observed ion=939.1 (M+H). 1H NMR (500 MHz, METHANOL-d4) δ ppm 8.70 (dd, J=2.53, 0.75 Hz, 1H), 8.45 (d, J=8.64 Hz, 1H), 8.37 (d, J=2.09 Hz, 1H), 8.18 (dd, J=8.79, 2.24 Hz, 1H), 7.32 (d, J=7.75 Hz, 1H), 7.24 (d, J=7.75 Hz, 1H), 6.99 (d, J=2.68 Hz, 1H), 6.54-6.85 (m, 4H), 4.48-4.59 (m, 2H), 3.64 (s, 3H), 3.50 (dd, J=14.16, 4.92 Hz, 1H), 3.26 (s, 3H), 3.13 (dd, J=14.01, 9.24 Hz, 1H), 2.38-2.50 (m, 2H), 1.34-1.40 (m, 1H), 0.99-1.04 (m, 1H).

### Preparation of Example 34: N—((S)-1-((3P)-3-(4-chloro-1-methyl-3-(methylsulfonamido)-1H-indazol-7-yl)-7-(1-(2,2-difluoroethyl)-1H-pyrazol-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

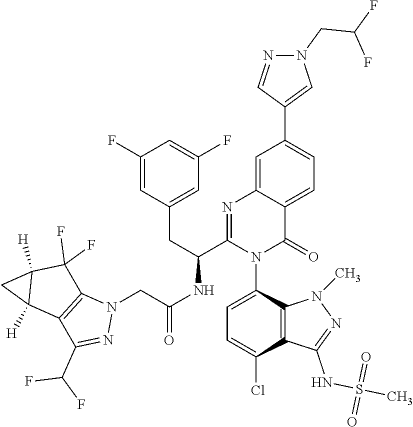

The title compound was prepared according to General Procedure K using 1-(2,2-difluoroethyl)-4-(4,4,5,5-tetramethyl-1,3,2-dioxaborolan-2-yl)-1H-pyrazole as the coupling partner. The experiment afforded the title compound, N—((S)-1-((3P)-3-(4-chloro-1-methyl-3-(methylsulfonamido)-1H-indazol-7-yl)-7-(1-(2,2-difluoroethyl)-1H-pyrazol-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.44 min.; observed ion=935.1 (M+H). 1H NMR (500 MHz, METHANOL-d4) δ ppm 8.37 (s, 1H), 8.28 (d, J=8.34 Hz, 1H), 8.17 (d, J=0.60 Hz, 1H), 8.08 (d, J=1.79 Hz, 1H), 7.90 (dd, J=8.34, 1.49 Hz, 1H), 7.30 (d, J=8.05 Hz, 1H), 7.18 (d, J=7.75 Hz, 1H), 6.58-6.83 (m, 4H), 6.16-6.42 (m, 1H), 4.84-4.86 (m, 1H), 4.69 (td, J=14.31, 3.87 Hz, 2H), 4.53 (s, 2H), 3.63 (s, 3H), 3.45-3.53 (m, 1H), 3.26 (s, 3H), 3.11 (dd, J=14.01, 9.24 Hz, 1H), 2.43 (ddd, J=11.18, 7.60, 4.17 Hz, 2H), 1.34-1.40 (m, 1H), 0.99-1.04 (m, 1H).

### Preparation of Example 35: N—((S)-1-((3P)-3-(4-chloro-1-methyl-3-(methylsulfonamido)-1H-indazol-7-yl)-7-(1-(difluoromethyl)-1H-pyrazol-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

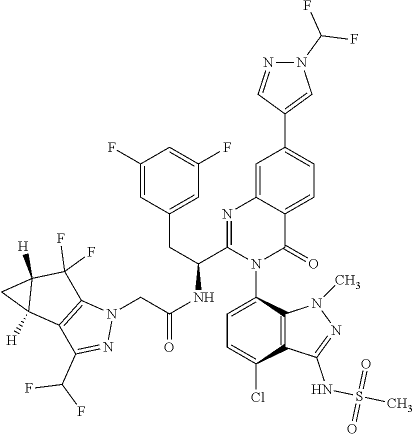

The title compound was prepared according to General Procedure K using 1-(difluoromethyl)-4-(4,4,5,5-tetramethyl-1,3,2-dioxaborolan-2-yl)-1H-pyrazole as the coupling partner. The experiment afforded the title compound, N—((S)-1-((3P)-3-(4-chloro-1-methyl-3-(methylsulfonamido)-1H-indazol-7-yl)-7-(1-(difluoromethyl)-1H-pyrazol-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.45 min.; observed ion=921.2 (M+H). 1H NMR (500 MHz, METHANOL-d4) δ ppm 8.75 (s, 1H), 8.34 (s, 1H), 8.32 (d, J=8.64 Hz, 1H), 8.16 (d, J=1.49 Hz, 1H), 7.97 (dd, J=8.34, 1.79 Hz, 1H), 7.47-7.76 (m, 1H), 7.31 (d, J=8.05 Hz, 1H), 7.18 (d, J=8.05 Hz, 1H), 6.56-6.85 (m, 4H), 4.52 (s, 2H), 3.63 (s, 3H), 3.45-3.53 (m, 1H), 3.26 (s, 3H), 3.12 (dd, J=14.01, 9.24 Hz, 1H), 2.43 (ddd, J=11.33, 7.60, 4.02 Hz, 2H), 1.34-1.41 (m, 1H), 0.97-1.05 (m, 1H).

### Preparation of Example 36: N—((S)-1-((3P)-3-(4-chloro-1-methyl-3-(methylsulfonamido)-1H-indazol-7-yl)-7-(2-methylbenzo[d]thiazol-5-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

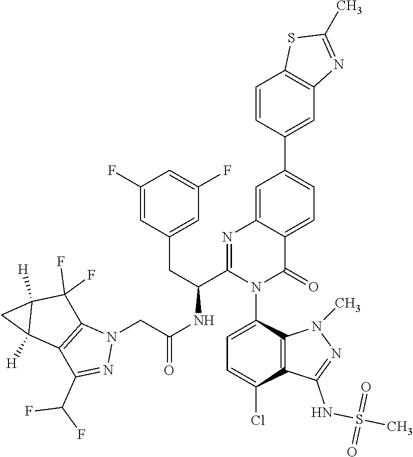

The title compound was prepared according to General Procedure K using (2-methylbenzo[d]thiazol-5-yl)boronic acid as the coupling partner. The experiment afforded the title compound, N—((S)-1-((3P)-3-(4-chloro-1-methyl-3-(methylsulfonamido)-1H-indazol-7-yl)-7-(2-methylbenzo[d]thiazol-5-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.59 min.; observed ion=952.1 (M+H). 1H NMR (500 MHz, METHANOL-d4) δ ppm 8.40 (d, J=8.34 Hz, 1H), 8.34 (d, J=1.79 Hz, 1H), 8.22 (d, J=1.79 Hz, 1H), 8.14 (d, J=8.34 Hz, 1H), 8.04 (dd, J=8.34, 1.79 Hz, 1H), 7.89 (dd, J=8.34, 1.79 Hz, 1H), 7.32 (d, J=7.75 Hz, 1H), 7.22 (d, J=7.75 Hz, 1H), 6.55-6.84 (m, 4H), 4.89-4.92 (m, 1H), 4.51-4.61 (m, 2H), 3.65 (s, 3H), 3.51 (dd, J=14.01, 5.07 Hz, 1H), 3.26 (s, 3H), 3.14 (dd, J=14.01, 9.24 Hz, 1H), 2.92 (s, 3H), 2.43 (ddd, J=11.62, 7.75, 4.17 Hz, 2H), 1.34-1.39 (m, 1H), 0.97-1.04 (m, 1H).

### Preparation of Example 37: N—((S)-1-((3P)-3-(4-chloro-1-methyl-3-(methylsulfonamido)-1H-indazol-7-yl)-7-(4-(1,1-difluoroethyl)pyridin-2-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

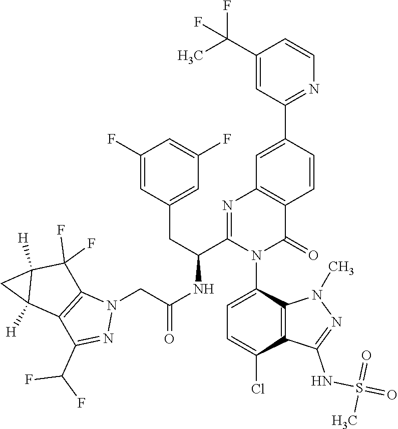

The title compound was prepared according to General Procedure L using 2-chloro-4-(1,1-difluoroethyl)pyridine as the coupling partner. The experiment afforded the title compound, N—((S)-1-((3P)-3-(4-chloro-1-methyl-3-(methylsulfonamido)-1H-indazol-7-yl)-7-(4-(1,1-difluoroethyl)pyridin-2-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.56 min.; observed ion=946.3 (M+H). 1H NMR (500 MHz, METHANOL-d4) δ ppm 8.88 (d, J=5.07 Hz, 1H), 8.55 (d, J=1.49 Hz, 1H), 8.39-8.44 (m, 1H), 8.34 (dd, J=8.34, 1.79 Hz, 1H), 8.21 (s, 1H), 7.64 (dd, J=5.07, 1.49 Hz, 1H), 7.15-7.35 (m, 2H), 6.58-6.81 (m, 4H), 4.53 (d, J=2.98 Hz, 2H), 3.61 (s, 3H), 3.49 (dd, J=14.16, 5.22 Hz, 1H), 3.22 (s, 3H), 3.11 (dd, J=14.01, 9.24 Hz, 1H), 2.37-2.46 (m, 2H), 2.05 (t, J=18.63 Hz, 3H), 1.32-1.37 (m, 1H), 0.97-1.02 (m, 1H).

### Preparation of Example 38: N—((S)-1-((3P)-3-(4-chloro-1-methyl-3-(methylsulfonamido)-1H-indazol-7-yl)-4-oxo-7-(4-(trifluoromethyl)-1H-pyrazol-1-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

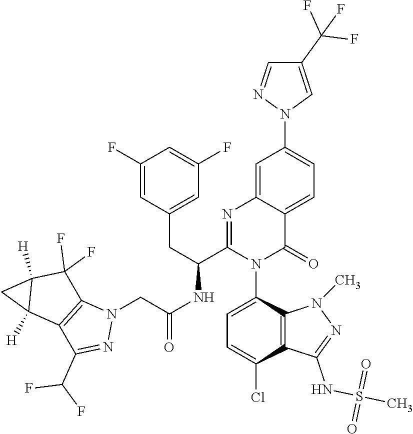

To a 5 mL microwave vial charged with N—((S)-1-(3-(4-chloro-1-methyl-3-(methylsulfonamido)-1H-indazol-7-yl)-4-oxo-7-(4,4,5,5-tetramethyl-1,3,2-dioxaborolan-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (40 mg, 0.043 mmol), 4-(trifluoromethyl)-1H-pyrazole (7.01 mg, 0.052 mmol), copper (II) acetate (2.341 mg, 0.013 mmol), boric acid (5.31 mg, 0.086 mmol) and powdered activated 4 A molecular sieves (25 mg) was added acetonitrile (2 mL). The mixture was stirred under air at 80° C. for 16 h. The mixture was cooled to room temperature, filtered and the filtrated was concentrated in vacuo. The residue was dissolved in DMF (2 mL), filtered, and the filtrate was subjected to HPLC purification to afford the title compound, N—((S)-1-((3P)-3-(4-chloro-1-methyl-3-(methylsulfonamido)-1H-indazol-7-yl)-4-oxo-7-(4-(trifluoromethyl)-1H-pyrazol-1-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.54 min.; observed ion=939.2 (M+H). 1H NMR (500 MHz, METHANOL-d4) δ ppm 9.09 (s, 1H), 8.41 (d, J=8.64 Hz, 1H), 8.37 (d, J=2.09 Hz, 1H), 8.13-8.18 (m, 2H), 7.29 (d, J=8.05 Hz, 1H), 7.20 (d, J=7.75 Hz, 1H), 6.53-6.81 (m, 4H), 4.50 (d, J=2.09 Hz, 2H), 3.61 (s, 3H), 3.48 (dd, J=14.01, 5.07 Hz, 1H), 3.23 (s, 3H), 3.10 (dd, J=13.86, 9.39 Hz, 1H), 2.37-2.45 (m, 2H), 1.32-1.37 (m, 1H), 0.96-1.01 (m, 1H).

### Preparation of Example 39: N—((S)-1-(7-(4-acetylpyrimidin-2-yl)-(3P)-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

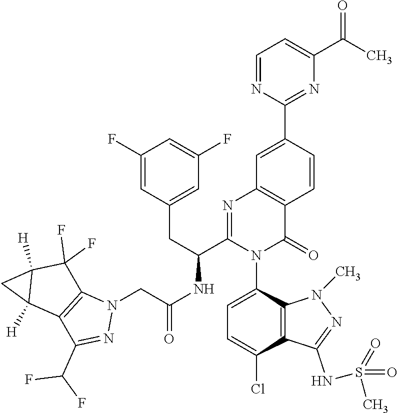

The title compound was prepared according to General Procedure L using 1-(2-chloropyrimidin-4-yl)ethan-1-one as the coupling partner. The experiment afforded the title compound, N—((S)-1-(7-(4-acetylpyrimidin-2-yl)-(3P)-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.49 min.; observed ion=925.4 (M+H). 1H NMR (500 MHz, METHANOL-d4) δ ppm 9.24 (d, J=5.07 Hz, 1H), 9.03-9.08 (m, 1H), 8.81 (dd, J=8.35, 1.49 Hz, 1H), 8.46 (dd, J=8.34, 0.60 Hz, 1H), 7.96 (d, J=5.07 Hz, 1H), 7.22-7.34 (m, 2H), 6.57-6.84 (m, 4H), 4.57 (d, J=5.66 Hz, 2H), 3.64 (s, 3H), 3.51 (dd, J=14.16, 4.92 Hz, 1H), 3.25 (s, 3H), 3.15 (dd, J=13.86, 9.39 Hz, 1H), 2.89 (s, 3H), 2.39-2.47 (m, 2H), 1.33-1.40 (m, 1H), 0.98-1.04 (m, 1H).

### Preparation of Example 40: N—((S)-1-((3P)-3-(4-chloro-1-methyl-3-(methylsulfonamido)-1H-indazol-7-yl)-7-(1-cyclopropyl-1H-pyrazol-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

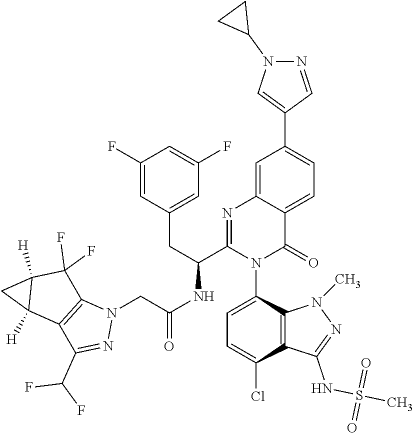

The title compound was prepared according to General Procedure K using 1-cyclopropyl-4-(4,4,5,5-tetramethyl-1,3,2-dioxaborolan-2-yl)-1H-pyrazole as the coupling partner. The experiment afforded the title compound, N—((S)-1-((3P)-3-(4-chloro-1-methyl-3-(methylsulfonamido)-1H-indazol-7-yl)-7-(1-cyclopropyl-1H-pyrazol-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.48 min.; observed ion=911.4 (M+H). 1H NMR (500 MHz, METHANOL-d4) δ ppm 8.34 (s, 1H), 8.19-8.27 (m, 1H), 7.99-8.08 (m, 2H), 7.86 (dd, J=8.20, 1.64 Hz, 1H), 7.27 (d, J=8.05 Hz, 1H), 7.14 (d, J=7.75 Hz, 1H), 6.54-6.83 (m, 4H), 4.50 (s, 2H), 3.73-3.79 (m, 1H), 3.60 (s, 3H), 3.43-3.50 (m, 1H), 3.22 (s, 3H), 3.08 (dd, J=13.86, 9.39 Hz, 1H), 2.35-2.46 (m, 2H), 1.33-1.37 (m, 1H), 1.18-1.22 (m, 2H), 1.08-1.14 (m, 2H), 0.95-1.02 (m, 1H).

### Preparation of Example 41: N—((S)-1-((3P)-3-(4-chloro-1-methyl-3-(methylsulfonamido)-1H-indazol-7-yl)-4-oxo-7-(1-(2,2,2-trifluoroethyl)-1H-pyrazol-4-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

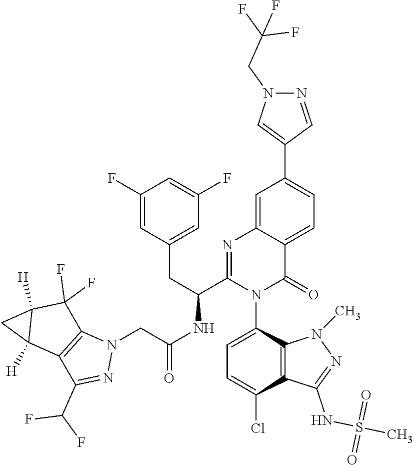

The title compound was prepared according to General Procedure K using 4-(4,4,5,5-tetramethyl-1,3,2-dioxaborolan-2-yl)-1-(2,2,2-trifluoroethyl)-1H-pyrazole as the coupling partner. The experiment afforded the title compound, N—((S)-1-((3P)-3-(4-chloro-1-methyl-3-(methylsulfonamido)-1H-indazol-7-yl)-4-oxo-7-(1-(2,2,2-trifluoroethyl)-1H-pyrazol-4-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.47 min.; observed ion=953 (M+H). 1H NMR (500 MHz, METHANOL-d4) δ ppm 8.41 (s, 1H), 8.25-8.30 (m, 1H), 8.18 (d, J=0.60 Hz, 1H), 8.07 (d, J=1.19 Hz, 1H), 7.89 (dd, J=8.34, 1.79 Hz, 1H), 7.14-7.31 (m, 2H), 6.51-6.82 (m, 4H), 5.02-5.09 (m, 2H), 4.50 (s, 2H), 3.61 (s, 3H), 3.44-3.51 (m, 1H), 3.23 (s, 3H), 3.09 (dd, J=13.86, 9.09 Hz, 1H), 2.36-2.46 (m, 2H), 1.32-1.37 (m, 1H), 0.96-1.03 (m, 1H).

### Preparation of Example 42: N—((S)-1-((3P)-3-(4-chloro-1-methyl-3-(methylsulfonamido)-1H-indazol-7-yl)-7-(1-(2-fluoroethyl)-1H-pyrazol-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

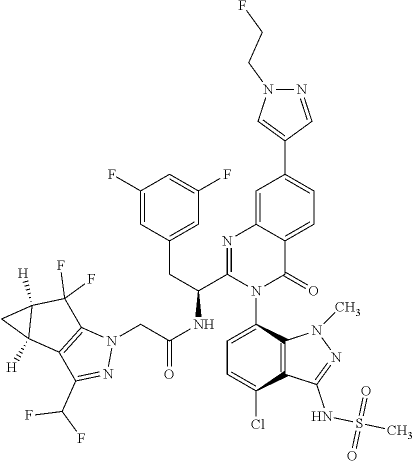

The title compound was prepared according to General Procedure K using 1-(2-fluoroethyl)-4-(4,4,5,5-tetramethyl-1,3,2-dioxaborolan-2-yl)-1H-pyrazole as the coupling partner. The experiment afforded the title compound, N—((S)-1-((3P)-3-(4-chloro-1-methyl-3-(methylsulfonamido)-1H-indazol-7-yl)-7-(1-(2-fluoroethyl)-1H-pyrazol-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.39 min.; observed ion=917.4 (M+H). Column: Waters XSelect CSH C18, 19×100 mm, 5 μm particles; Solvent A=0.1% Formic Acid in 100% Water. Solvent B=Acetonitrile. Flow Rate=40 ml/min. start % B=49. Final % B=69. Gradient Time=6 min, then a 2 min hold at 98% B. Wavelength=215 and 254 nm. ESI+ Range: 150 to 2000 dalton. Sample was loaded at Start B % for 1 min.

### Preparation of Example 43: N—((S)-1-((3P)-3-(4-chloro-1-methyl-3-(methylsulfonamido)-1H-indazol-7-yl)-7-(1-cyclopropyl-1H-pyrazol-5-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

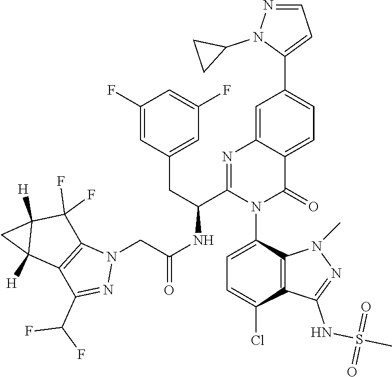

The title compound was prepared according to General Procedure K using 1-cyclopropyl-5-(4,4,5,5-tetramethyl-1,3,2-dioxaborolan-2-yl)-1H-pyrazole as the coupling partner. The experiment afforded the title compound, N—((S)-1-((3P)-3-(4-chloro-1-methyl-3-(methylsulfonamido)-1H-indazol-7-yl)-7-(1-cyclopropyl-1H-pyrazol-5-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.45 min.; observed ion=911.4 (M+H). 1H NMR (500 MHz, METHANOL-d4) δ ppm 8.28-8.42 (m, 1H), 8.14 (d, J=1.19 Hz, 1H), 7.93 (dd, J=8.34, 1.79 Hz, 1H), 7.56 (d, J=1.79 Hz, 1H), 7.26-7.33 (m, 1H), 7.22 (d, J=8.05 Hz, 1H), 6.71 (s, 5H), 4.51 (d, J=1.79 Hz, 2H), 3.80-3.90 (m, 1H), 3.62 (s, 3H), 3.42-3.50 (m, 1H), 3.23 (s, 3H), 3.09 (dd, J=14.01, 9.24 Hz, 1H), 2.35-2.47 (m, 2H), 1.31-1.38 (m, 1H), 1.01-1.10 (m, 4H), 0.96-1.00 (m, 1H).

### Preparation of Example 44: N—((S)-1-(7-(benzo[d]thiazol-6-yl)-(3P)-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

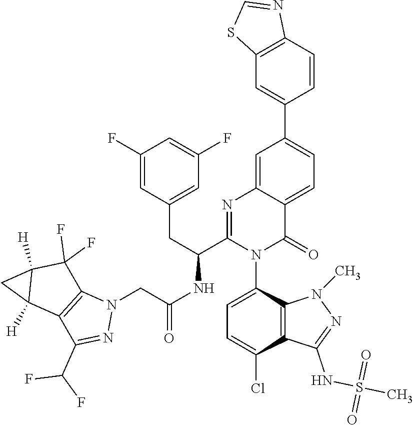

The title compound was prepared according to General Procedure K using benzo[d]thiazol-6-ylboronic acid as the coupling partner. The experiment afforded the title compound, N—((S)-1-(7-(benzo[d]thiazol-6-yl)-(3P)-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.5 min.; observed ion=938.3 (M+H). 1H NMR (500 MHz, METHANOL-d4) δ ppm 9.35 (s, 1H), 8.57 (d, J=1.49 Hz, 1H), 8.39 (d, J=8.34 Hz, 1H), 8.21-8.28 (m, 2H), 8.04 (ddd, J=8.49, 4.62, 1.79 Hz, 2H), 7.16-7.31 (m, 2H), 6.55-6.83 (m, 4H), 4.52 (d, J=2.98 Hz, 2H), 3.62 (s, 3H), 3.46-3.53 (m, 1H), 3.23 (s, 3H), 3.11 (dd, J=14.01, 9.24 Hz, 1H), 2.35-2.48 (m, 2H), 1.31-1.36 (m, 1H), 0.96-1.03 (m, 1H).

### Preparation of Example 45: N—((S)-1-(7-(benzo[d]thiazol-5-yl)-(3P)-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

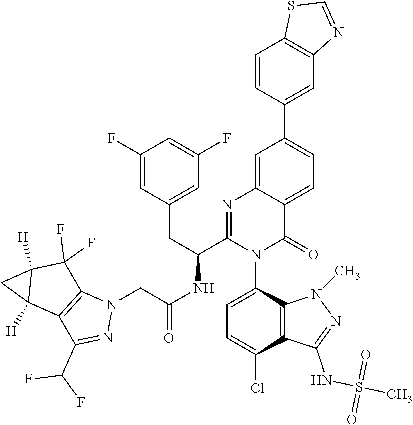

The title compound was prepared according to General Procedure K using benzo[d]thiazol-5-ylboronic acid as the coupling partner. The experiment afforded the title compound, N—((S)-1-(7-(benzo[d]thiazol-5-yl)-(3P)-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.53 min.; observed ion=938.4 (M+H). 1H NMR (500 MHz, METHANOL-d4) δ ppm 9.37 (s, 1H), 8.51 (d, J=1.49 Hz, 1H), 8.40 (d, J=8.64 Hz, 1H), 8.28 (d, J=8.05 Hz, 1H), 8.24 (d, J=1.49 Hz, 1H), 8.06 (dd, J=8.34, 1.79 Hz, 1H), 7.98 (dd, J=8.34, 1.79 Hz, 1H), 7.19-7.33 (m, 2H), 6.56-6.81 (m, 4H), 4.53 (d, J=4.17 Hz, 2H), 3.62 (s, 3H), 3.49 (dd, J=14.16, 4.92 Hz, 1H), 3.23 (s, 3H), 3.09-3.15 (m, 1H), 2.37-2.46 (m, 2H), 1.32-1.37 (m, 1H), 0.96-1.03 (m, 1H).

### Preparation of Example 46: N—((S)-1-(7-(benzo[d]oxazol-5-yl)-(3P)-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

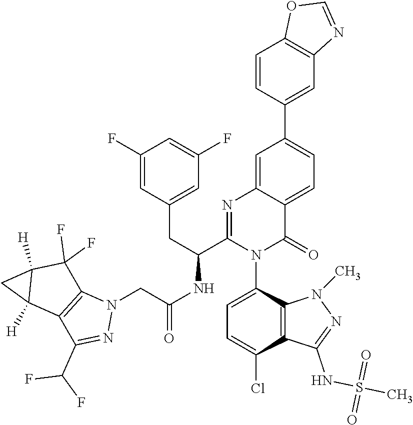

The title compound was prepared according to General Procedure K using benzo[d]oxazol-5-ylboronic acid as the coupling partner. The experiment afforded the title compound, N—((S)-1-(7-(benzo[d]oxazol-5-yl)-(3P)-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.48 min.; observed ion=922.3 (M+H). 1H NMR (500 MHz, METHANOL-d4) δ ppm 8.59 (s, 1H), 8.38 (d, J=8.34 Hz, 1H), 8.18 (dd, J=12.67, 1.64 Hz, 2H), 8.00 (dd, J=8.20, 1.94 Hz, 1H), 7.91-7.95 (m, 1H), 7.84-7.89 (m, 1H), 7.30 (d, J=7.75 Hz, 1H), 7.20 (d, J=7.75 Hz, 1H), 6.56-6.81 (m, 5H), 4.49-4.57 (m, 2H), 3.62 (s, 3H), 3.49 (dd, J=14.01, 5.07 Hz, 1H), 3.23 (s, 3H), 3.11 (dd, J=13.71, 9.24 Hz, 1H), 2.36-2.47 (m, 2H), 1.32-1.37 (m, 1H), 0.96-1.01 (m, 1H).

### Preparation of Example 47: N—((S)-1-((3P)-3-(4-chloro-1-methyl-3-(methylsulfonamido)-1H-indazol-7-yl)-4-oxo-7-(1-(2,2,3,3-tetrafluoropropyl)-1H-pyrazol-3-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

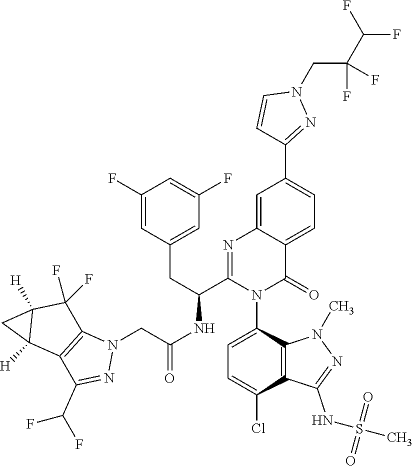

The title compound was prepared according to General Procedure M using 2,2,3,3-tetrafluoropropyl trifluoromethanesulfonate as the coupling partner. The experiment afforded the title compound, N—((S)-1-((3P)-3-(4-chloro-1-methyl-3-(methylsulfonamido)-1H-indazol-7-yl)-4-oxo-7-(1-(2,2,3,3-tetrafluoropropyl)-1H-pyrazol-3-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.48 min.; observed ion=985.4 (M+H). 1H NMR (500 MHz, METHANOL-d4) δ ppm 8.30-8.37 (m, 2H), 8.15 (dd, J=8.34, 1.79 Hz, 1H), 7.90 (d, J=2.38 Hz, 1H), 7.31 (d, J=7.75 Hz, 1H), 7.21 (d, J=7.75 Hz, 1H), 7.01 (d, J=2.38 Hz, 1H), 6.59-6.83 (m, 4H), 6.16-6.42 (m, 1H), 4.96-5.02 (m, 2H), 4.50-4.59 (m, 2H), 3.64 (s, 3H), 3.49 (dd, J=14.01, 5.07 Hz, 1H), 3.26 (s, 3H), 3.12 (dd, J=14.01, 9.24 Hz, 1H), 2.37-2.48 (m, 2H), 1.34-1.40 (m, 1H), 0.98-1.05 (m, 1H).

### Preparation of Example 48: N—((S)-1-((3P)-3-(4-chloro-1-methyl-3-(methylsulfonamido)-1H-indazol-7-yl)-4-oxo-7-(1-(2,2,3,3,3-pentafluoropropyl)-1H-pyrazol-3-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

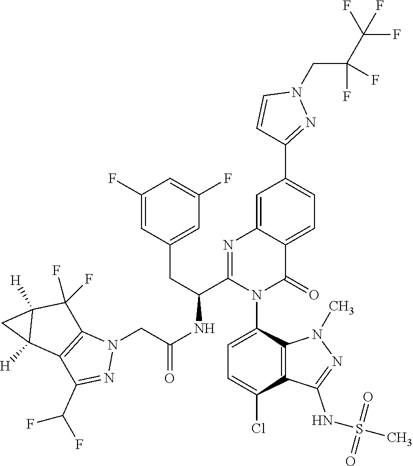

The title compound was prepared according to General Procedure M using 2,2,3,3,3-pentafluoropropyl trifluoromethanesulfonate as the coupling partner. The experiment afforded the title compound, N—((S)-1-((3P)-3-(4-chloro-1-methyl-3-(methylsulfonamido)-1H-indazol-7-yl)-4-oxo-7-(1-(2,2,3,3,3-pentafluoropropyl)-1H-pyrazol-3-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.54 min.; observed ion=1003.4 (M+H). 1H NMR (500 MHz, METHANOL-d4) δ ppm 8.30-8.36 (m, 2H), 8.11-8.17 (m, 1H), 7.93 (d, J=2.68 Hz, 1H), 7.31 (d, J=7.75 Hz, 1H), 7.21 (d, J=7.75 Hz, 1H), 7.03 (d, J=2.68 Hz, 1H), 6.59-6.82 (m, 4H), 5.16 (t, J=14.60 Hz, 2H), 4.50-4.59 (m, 2H), 3.64 (s, 3H), 3.49 (dd, J=13.86, 4.92 Hz, 1H), 3.26 (s, 3H), 3.12 (dd, J=14.01, 9.24 Hz, 1H), 2.38-2.49 (m, 2H), 1.34-1.40 (m, 1H), 0.98-1.05 (m, 1H).

### Preparation of Example 49: N—((S)-1-((3P)-3-(4-chloro-1-methyl-3-(methylsulfonamido)-1H-indazol-7-yl)-7-(1-(2,2-difluorobutyl)-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

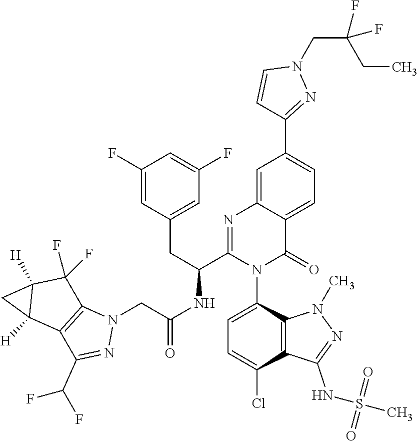

The title compound was prepared according to General Procedure M using 2,2-difluorobutyl trifluoromethanesulfonate as the coupling partner. The experiment afforded the title compound, N—((S)-1-((3P)-3-(4-chloro-1-methyl-3-(methylsulfonamido)-1H-indazol-7-yl)-7-(1-(2,2-difluorobutyl)-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.52 min.; observed ion=963.4 (M+H). 1H NMR (500 MHz, METHANOL-d4) δ ppm 8.30-8.34 (m, 2H), 8.13 (dd, J=8.34, 1.49 Hz, 1H), 7.84 (d, J=2.38 Hz, 1H), 7.31 (d, J=7.75 Hz, 1H), 7.21 (d, J=8.05 Hz, 1H), 6.98 (d, J=2.38 Hz, 1H), 6.59-6.82 (m, 4H), 4.72 (t, J=12.96 Hz, 2H), 4.50-4.58 (m, 2H), 3.64 (s, 3H), 3.49 (dd, J=14.01, 5.07 Hz, 1H), 3.26 (s, 3H), 3.12 (dd, J=14.01, 9.24 Hz, 1H), 2.39-2.48 (m, 2H), 1.89-2.01 (m, 2H), 1.34-1.40 (m, 1H), 1.14 (t, J=7.45 Hz, 3H), 0.99-1.05 (m, 1H).

### Preparation of Example 50: N—((S)-1-((3P)-3-(4-chloro-1-methyl-3-(methylsulfonamido)-1H-indazol-7-yl)-4-oxo-7-(1-(3,3,3-trifluoropropyl)-1H-pyrazol-3-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

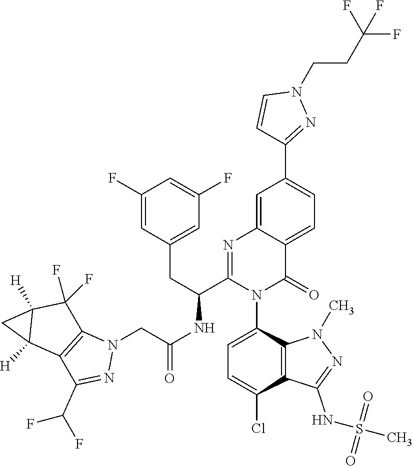

The title compound was prepared according to General Procedure M using 3,3,3-trifluoropropyl trifluoromethanesulfonate as the coupling partner. The experiment afforded the title compound, N—((S)-1-((3P)-3-(4-chloro-1-methyl-3-(methylsulfonamido)-1H-indazol-7-yl)-4-oxo-7-(1-(3,3,3-trifluoropropyl)-1H-pyrazol-3-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.49 min.; observed ion=967 (M+H). 1H NMR (500 MHz, METHANOL-d4) δ ppm 8.30-8.34 (m, 2H), 8.10-8.15 (m, 1H), 7.84 (d, J=2.38 Hz, 1H), 7.31 (d, J=7.75 Hz, 1H), 7.21 (d, J=7.75 Hz, 1H), 6.91 (d, J=2.38 Hz, 1H), 6.59-6.84 (m, 4H), 4.57 (t, J=7.00 Hz, 2H), 4.54 (d, J=3.28 Hz, 2H), 3.64 (s, 3H), 3.49 (dd, J=14.16, 5.22 Hz, 1H), 3.26 (s, 3H), 3.09-3.14 (m, 1H), 2.90-3.00 (m, 2H), 2.38-2.50 (m, 2H), 1.34-1.40 (m, 1H), 0.98-1.05 (m, 1H).

### Preparation of Example 51: N—((S)-1-((3P)-3-(4-chloro-1-methyl-3-(methylsulfonamido)-1H-indazol-7-yl)-7-(1-(3-fluoropropyl)-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

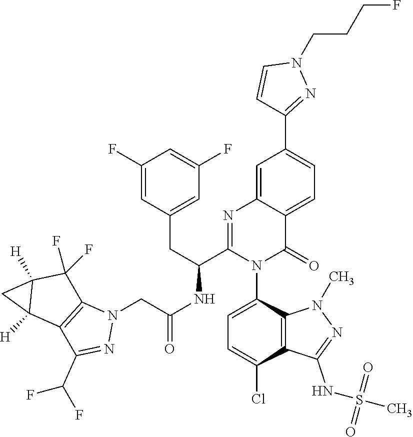

The title compound was prepared according to General Procedure M using 3-fluoropropyl trifluoromethanesulfonate as the coupling partner. The experiment afforded the title compound, N—((S)-1-((3P)-3-(4-chloro-1-methyl-3-(methylsulfonamido)-1H-indazol-7-yl)-7-(1-(3-fluoropropyl)-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.44 min.; observed ion=931 (M+H). 1H NMR (500 MHz, METHANOL-d4) δ ppm 8.30-8.35 (m, 2H), 8.13 (dd, J=8.34, 1.79 Hz, 1H), 7.83 (d, J=2.38 Hz, 1H), 7.32 (d, J=8.05 Hz, 1H), 7.21 (d, J=7.75 Hz, 1H), 6.93 (d, J=2.38 Hz, 1H), 6.60-6.83 (m, 4H), 4.60 (d, J=5.07 Hz, 2H), 4.51-4.55 (m, 4H), 3.64 (s, 3H), 3.50 (dd, J=14.01, 5.07 Hz, 1H), 3.26 (s, 3H), 3.10-3.14 (m, 1H), 2.40-2.48 (m, 2H), 1.33-1.40 (m, 1H), 0.98-1.05 (m, 1H).

### Preparation of Example 52: N—((S)-1-((3P)-3-(4-chloro-1-methyl-3-(methylsulfonamido)-1H-indazol-7-yl)-7-(1-(3,3-difluorobutyl)-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

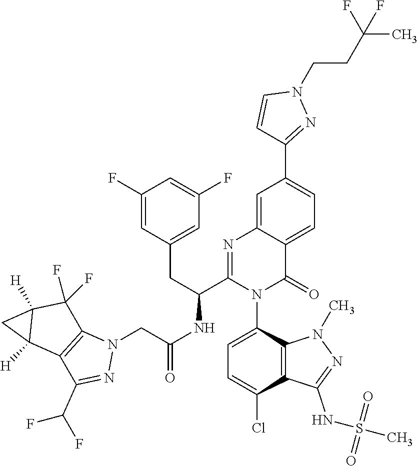

The title compound was prepared according to General Procedure M using 3,3-difluorobutyl trifluoromethanesulfonate as the coupling partner. The experiment afforded the title compound, N—((S)-1-((3P)-3-(4-chloro-1-methyl-3-(methylsulfonamido)-1H-indazol-7-yl)-7-(1-(3,3-difluorobutyl)-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.53 min.; observed ion=963.4 (M+H). Column: Waters XSelect CSH C18, 19×100 mm, 5 μm particles; Solvent A=0.1% Formic Acid in 100% Water. Solvent B=Acetonitrile. Flow Rate=40 mL/min. start % B=56.6. Final % B=76.6. Gradient Time=6 min, then a 2 min hold at 98% B. Wavelength=215 and 254 nm. ESI+ Range: 150 to 2000 dalton. Sample was loaded at Start B % for 1 min.

### Preparation of Example 53: N—((S)-1-((3P)-3-(4-chloro-1-methyl-3-(methylsulfonamido)-1H-indazol-7-yl)-4-oxo-7-(1-(4,4,4-trifluorobutyl)-1H-pyrazol-3-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

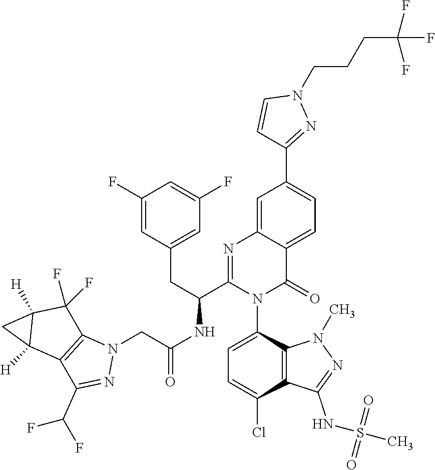

The title compound was prepared according to General Procedure M using 4,4,4-trifluorobutyl trifluoromethanesulfonate as the coupling partner. The experiment afforded the title compound, N—((S)-1-((3P)-3-(4-chloro-1-methyl-3-(methylsulfonamido)-1H-indazol-7-yl)-4-oxo-7-(1-(4,4,4-trifluorobutyl)-1H-pyrazol-3-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.55 min.; observed ion=981.4 (M+H). 1H NMR (500 MHz, METHANOL-d4) δ ppm 8.30-8.34 (m, 2H), 8.12 (dd, J=8.49, 1.64 Hz, 1H), 7.81 (d, J=2.38 Hz, 1H), 7.31 (d, J=8.05 Hz, 1H), 7.21 (d, J=7.75 Hz, 1H), 6.92 (d, J=2.38 Hz, 1H), 6.61-6.82 (m, 4H), 4.49-4.59 (m, 2H), 4.38 (t, J=6.26 Hz, 2H), 3.64 (s, 3H), 3.50 (dd, J=14.31, 5.07 Hz, 1H), 3.26 (s, 3H), 3.13 (dd, J=13.56, 4.32 Hz, 1H), 2.39-2.47 (m, 2H), 2.20-2.30 (m, 4H), 1.34-1.39 (m, 1H), 0.99-1.04 (m, 1H).

### Preparation of Example 54: N—((S)-1-((3P)-3-(4-chloro-1-methyl-3-(methylsulfonamido)-1H-indazol-7-yl)-4-oxo-7-(1-(2-(trifluoromethoxy)ethyl)-1H-pyrazol-3-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

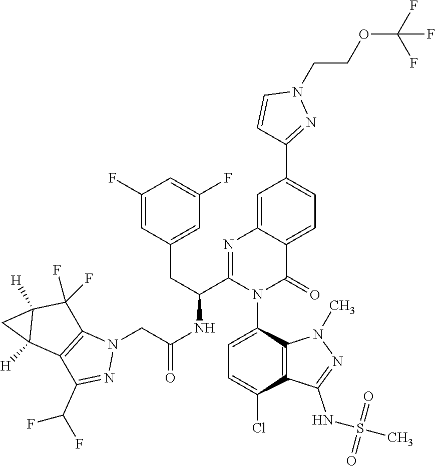

The title compound was prepared according to General Procedure M using 2-(trifluoromethoxy)ethyl trifluoromethanesulfonate as the coupling partner. The experiment afforded the title compound, N—((S)-1-((3P)-3-(4-chloro-1-methyl-3-(methylsulfonamido)-1H-indazol-7-yl)-4-oxo-7-(1-(2-(trifluoromethoxy)ethyl)-1H-pyrazol-3-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.5 min.; observed ion=983.4 (M+H). 1H NMR (500 MHz, METHANOL-d4) δ ppm 8.30-8.35 (m, 2H), 8.13 (dd, J=8.34, 1.79 Hz, 1H), 7.83 (d, J=2.38 Hz, 1H), 7.32 (d, J=8.05 Hz, 1H), 7.21 (d, J=7.75 Hz, 1H), 6.93 (d, J=2.38 Hz, 1H), 6.60-6.83 (m, 4H), 4.60 (d, J=5.07 Hz, 2H), 4.51-4.55 (m, 4H), 3.64 (s, 3H), 3.50 (dd, J=14.01, 5.07 Hz, 1H), 3.26 (s, 3H), 3.10-3.14 (m, 1H), 2.40-2.48 (m, 2H), 1.33-1.40 (m, 1H), 0.98-1.05 (m, 1H).

### Preparation of 3-bromo-1-(2,2,3,3-tetrafluoropropyl)-1H-pyrazole

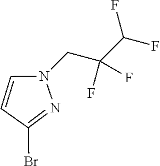 [SMILES: FC(F)C(F)(F)Cn1ccc(Br)n1]

To a solution of 3-bromo-1H-pyrazole (1 g, 6.80 mmol) and 2,2,3,3-tetrafluoropropyl trifluoromethanesulfonate (1.977 g, 7.48 mmol) in N,N-Dimethylformamide (DMF) (30 mL) was added K₂CO₃ (1.410 g, 10.21 mmol) and the resulting mixture was stirred at room temp for 16 h. The mixture was diluted with water and then was extracted with ether; washed with brine; dried (Na₂SO₄); filtered and the filtrate was concentrated under reduced pressure to afford 3-bromo-1-(2,2,3,3-tetrafluoropropyl)-1H-pyrazole (1.5 g, 84% yield) as light yellow liquid. ¹H NMR (500 MHz, CHLOROFORM-d) δ ppm 7.43 (d, J=2.38 Hz, 1H), 6.40 (d, J=2.68 Hz, 1H), 5.68-6.04 (m, 1H), 4.66 (tt, J=13.30, 1.30 Hz, 2H).

### Preparation of 1-(2,2-difluoropropyl)-3-(tributylstannyl)-1H-pyrazole

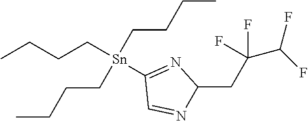 [SMILES: CCC[CH2][Sn]([CH2]CCC)([CH2]CCC)[C]1=NC(CC(F)(F)C(F)F)N=C1]

To a sealed tube charged with 3-bromo-1-(2,2,3,3-tetrafluoropropyl)-1H-pyrazole (1.6 g, 6.13 mmol), 1,1,1,2,2,2-hexabutyldistannane (6.20 mL, 12.26 mmol) and tetrakis(triphenylphosphine)palladium(0) (0.708 g, 0.613 mmol) under Ar was added toluene (30 mL). The mixture was degassed (brief high vacuum, then refilled with Ar) and then heated at 110° C. for 16 h. The reaction mixture was cooled to room temperature and then was diluted with EtOAc and washed with water and brine. The organic phase was concentrated onto Celite under reduced pressure and the resulting powder was subjected to silica gel chromatography eluting with 0-15% EtOAc in hexane to afford the title compound 1-(2,2,3,3-tetrafluoropropyl)-3-(tributylstannyl)-1H-pyrazole (600 mg, 21% yield) as a clear viscous oil contaminated with triphenylphosphine. LCMS Method: Column=Acquity UPLC BEH C18, 2.1×100 mm, 1.7 μm particles; Solvent A=0.1% Formic acid in 95:5 Water:MeCN; Solvent B=0.1% Formic Acid in 5:95 Water:MeCN; Flow Rate=0.8 mL/min; Gradient Profile (time/% B)=0 min/0%, 3.5 min/100%, 5.5 min/100%.; Detection wavelength=220 and 254 nm. LCMS result: retention time=4.02 min; m/z=473.05 [M+H]⁺. The product was used in subsequent chemical steps without further purification.

### Preparation of 1-(2,2-difluoropropyl)-3-(tributylstannyl)-1H-pyrazole

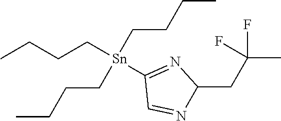 [SMILES: CCC[CH2][Sn]([CH2]CCC)([CH2]CCC)[C]1=NC(CC(C)(F)F)N=C1]

To a sealed tube charged with 3-bromo-1-(2,2-difluoropropyl)-1H-pyrazole (370 mg, 1.644 mmol), 1,1,1,2,2,2-hexabutyldistannane (1.662 mL, 3.29 mmol), and tetrakis(triphenylphosphine)palladium(0) (190 mg, 0.164 mmol) under Ar with was added toluene (10 mL). The reaction solution was degassed with Ar and then was stir at 110° C. overnight. The reaction solution was cooled to room temperature. The solution was diluted with EtOAc and then was washed with water followed by brine. The organic phase was concentrated onto Celite under reduced pressure and the resulting powder was subjected to silica gel chromatography (40 g column) eluting with 0-15% ethyl acetate in hexanes to afford 1-(2,2-difluoropropyl)-3-(tributylstannyl)-1H-pyrazole (320 mg, 45% yield) as a clear viscous oil contaminated with triphenylphosphine. The product was used in subsequent chemical steps without further purification.

### Preparation of(S)—N-((6P)-7-(2-(1-amino-2-(3,5-difluorophenyl)ethyl)-7-(1-(2,2-difluoropropyl)-1H-pyrazol-3-yl)-4-oxoquinazolin-3(4H)-yl)-4-chloro-1-methyl-1H-indazol-3-yl)methanesulfonamide

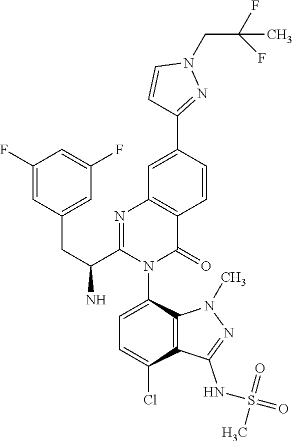

To a mixture of (S)—N-((6P)-7-(2-(1-amino-2-(3,5-difluorophenyl)ethyl)-7-bromo-4-oxoquinazolin-3(4H)-yl)-4-chloro-1-methyl-1H-indazol-3-yl)methanesulfonamide (400 mg, 0.627 mmol), 1-(2,2-difluoropropyl)-3-(tributylstannyl)-1H-pyrazole (409 mg, 0.941 mmol) and copper(I) iodide (11.94 mg, 0.063 mmol) in N,N-Dimethylformamide (DMF) (5 mL) was added tetrakis(triphenylphosphine)palladium(0) (72.5 mg, 0.063 mmol). The mixture was then degassed (brief high vacuum, then refilled with Ar) and heated at 100° C. for 16 h. After cooling to room temp, water was added and the mixture was extracted with ethyl acetate; washed with brine; dried (Na₂SO₄); filtered and the filtrate was concentrated in vacuo. The residue was subjected to silica gel chromatography eluting with 5-100% EtOAc in hexanes to afford the title compound (S)—N-((6P)-7-(2-(1-amino-2-(3,5-difluorophenyl)ethyl)-7-(1-(2,2-difluoropropyl)-1H-pyrazol-3-yl)-4-oxoquinazolin-3(4H)-yl)-4-chloro-1-methyl-1H-indazol-3-yl)methanesulfonamide (220 mg, 50% yield) as light yellow solid. ¹H NMR (500 MHz, DMSO-d₆) δ ppm 8.23 (br d, J=11.62 Hz, 2H), 8.10 (br d, J=8.34 Hz, 1H), 7.92-7.98 (m, 1H), 7.60-7.67 (m, 1H), 7.54-7.58 (m, 1H), 7.35 (s, 2H), 7.13 (d, J=2.09 Hz, 1H), 7.02 (br t, J=9.39 Hz, 1H), 6.76 (br d, J=6.85 Hz, 2H), 4.79 (br t, J=13.41 Hz, 2H), 3.70 (s, 3H), 3.53-3.59 (m, 1H), 3.23 (s, 3H), 2.85 (br dd, J=13.41, 8.34 Hz, 1H), 1.70 (t, J=19.07 Hz, 3H). LCMS Method: Column=Acquity UPLC BEH C18, 2.1×100 mm, 1.7 μm particles; Solvent A=0.1% Formic acid in 95:5 Water:MeCN; Solvent B=0.1% Formic Acid in 5:95 Water:MeCN; Flow Rate=0.8 mL/min; Gradient Profile (time/% B)=0 min/0%, 3.5 min/100%, 4.5 min/100%.; Detection wavelength=220 and 254 nm. LCMS Result: retention time=2.20 min; m/z=703.05 [M+H]⁺.

### Preparation of(S)—N-((6P)-7-(2-(1-amino-2-(3,5-difluorophenyl)ethyl)-4-oxo-7-(1-(2,2,3,3-tetrafluoropropyl)-1H-pyrazol-3-yl)quinazolin-3(4H)-yl)-4-chloro-1-methyl-1H-indazol-3-yl)methanesulfonamide

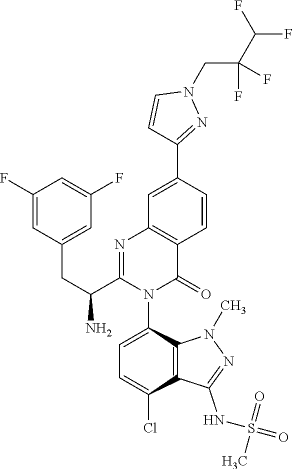

To a mixture of (S)—N-((6P)-7-(2-(1-amino-2-(3,5-difluorophenyl)ethyl)-7-bromo-4-oxoquinazolin-3(4H)-yl)-4-chloro-1-methyl-1H-indazol-3-yl)methanesulfonamide (500 mg, 0.784 mmol), 1-(2,2,3,3-tetrafluoropropyl)-3-(tributylstannyl)-1H-pyrazole (554 mg, 1.176 mmol) and copper(I) iodide (14.93 mg, 0.078 mmol) in N,N-Dimethylformamide (DMF) (5 mL) was added tetrakis(triphenylphosphine)palladium(0) (91 mg, 0.078 mmol). The mixture was degassed (brief high vacuum, then refilled with Ar) and then heated at 100° C. for 16 h. After cooling to room temp, water was added and the mixture was extracted with ethyl acetate; washed with brine; dried (Na₂SO₄); filtered and the filtrate was concentrated in vacuo. The resulting residue was subjected to silica gel chromatography eluting with 5-100% EtOAc in hexanes to afford the title compound (S)—N-((6P)-7-(2-(1-amino-2-(3,5-difluorophenyl)ethyl)-4-oxo-7-(1-(2,2,3,3-tetrafluoropropyl)-1H-pyrazol-3-yl)quinazolin-3(4H)-yl)-4-chloro-1-methyl-1H-indazol-3-yl)methanesulfonamide (220 mg, 38% yield) as light yellow solid.). LCMS Method: Column=Acquity UPLC BEH C18, 2.1×100 mm, 1.7 μm particles; Solvent A=0.1% Formic acid in 95:5 Water:MeCN; Solvent B=0.1% Formic Acid in 5:95 Water:MeCN; Flow Rate=0.8 mL/min; Gradient Profile (time/% B)=0 min/0%, 3.5 min/100%, 4.5 min/100%.; Detection wavelength=220 and 254 nm. LCMS result: retention time=2.37 min; m/z=739.15 [M+H]⁺.

### Preparation of N—((S)-1-((3P)-3-(4-chloro-3-(N-(4-methoxybenzyl)methylsulfonamido)-1-methyl-1H-indazol-7-yl)-7-(5-methyl-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

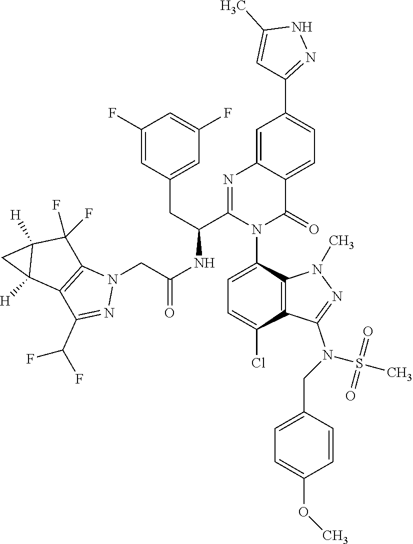

To a solution of N—((S)-1-((3P)-7-bromo-3-(4-chloro-3-(N-(4-methoxybenzyl)methylsulfonamido)-1-methyl-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (300 mg, 0.299 mmol), (5-methyl-1H-pyrazol-3-yl)boronic acid (113 mg, 0.896 mmol) and K₃PO₄ (190 mg, 0.896 mmol) in Tetrahydrofuran (THF) (4 mL)/Water (1.000 mL) was added Dichloro[9,9-dimethyl-4,5-bis(diphenylphosphino)xanthene]palladium(II) (22.58 mg, 0.030 mmol) and the resulting mixture was heated at 50° C. for 2 h. The mixture was then cooled to room temp; diluted with ethyl acetate and washed with water; dried (Na₂SO₄); filtered; and the filtrate was concentrated under reduced pressure. The resulting residue was subjected to silica gel chromatography (5-100% EtOAC in hexanes) to afford N—((S)-1-((3P)-3-(4-chloro-3-(N-(4-methoxybenzyl)methylsulfonamido)-1-methyl-1H-indazol-7-yl)-7-(5-methyl-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (240 mg, 0.239 mmol, 80% yield). LCMS Method: Column=Acquity UPLC BEH C18, 2.1×100 mm, 1.7 μm particles; Solvent A=95:5 Water:MeCN w/ 0.1% v/v Formic acid; Solvent B=5:95 Water:MeCN w/ 0.1% v/v Formic acid; Flow rate=0.80 ml/min; Gradient Profile (time/% B)=0 min/0%, 3.5 min/100%, 4.5 min/100%; Detection wavelength=220 and 254 nm. LC/MS result: retention time=3.74 min; m/z=1005.14 [M+H]⁺.

### Preparation of N-(7-amino-4-chloro-1-(3-fluoropropyl)-1H-indazol-3-yl)-N-(4-methoxybenzyl)methanesulfonamide

### Synthesis Scheme

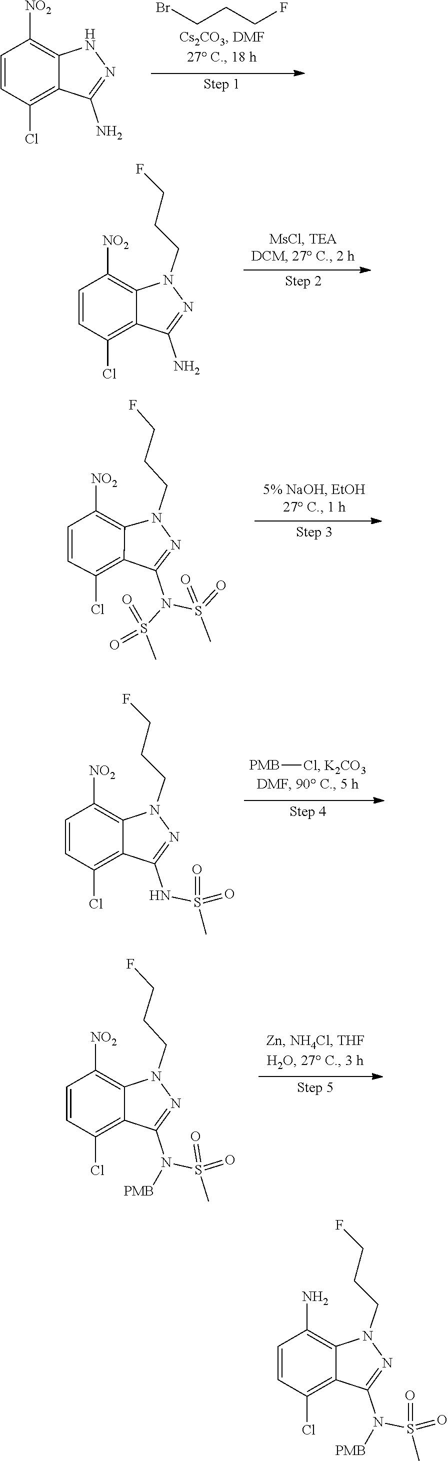

### Step 1: Preparation of 4-Chloro-1-(3-fluoropropyl)-7-nitro-1H-indazol-3-amine

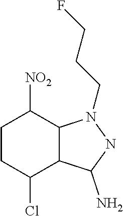 [SMILES: NC1NN(CCCF)C2C1C(Cl)CCC2[N+](=O)[O-]]

To a stirred solution of 4-chloro-7-nitro-1H-indazol-3-amine (12 g, 56.4 mmol) in DMF (10 mL) at 0° C. were added cesium carbonate (92 g, 282 mmol) followed by 1-bromo-3-fluoropropane (6.37 mL, 67.7 mmol). The reaction mass was allowed to warm to 27° C. and was stirred for 18 hrs. The progress of the reaction was monitored by TLC (SiO₂, 50% EtOAc/Pet. Rf=0.7). On completion, the reaction mixture was diluted with water (500 mL) and extracted with EtOAc (2×500 mL). The combined organics were washed with water (500 mL) and then brine (200 mL). The organic layer was dried over anhydrous Na₂SO₄, filtered, and the filtrate was concentrated under reduced pressure to afford the crude (18 g) as a yellow semi-solid. This material was purified via silica gel chromatography eluting with 10-50% EtOAc/Pet to afford 4-chloro-1-(3-fluoropropyl)-7-nitro-1H-indazol-3-amine as a red solid, 6 g (39% Yield). ¹H-NMR (400 MHz, DMSO-d₆) δ: 8.07 (d, J=8.4 Hz, 1H), 7.13 (d, J=8.4 Hz, 1H), 5.81 (s, 2H), 4.45 (t, J=5.6 Hz, 1H), 4.34-4.26 (m, 3H), 2.13-1.99 (m, 2H). LCMS Method: Column=Acquity BEH C18 (50 mm×2.1 mm, 1.7 um particles); Mobile Phase=A: 0.05% Formic Acid in water; Mobile B=0.05% Formic Acid in acetonitrile; Gradient profile (time (minutes)/% B): 0/3, 0.4/3, 2.5/98, 3.4/98, 3.5/3, 4/3; Column Temp=35° C.; Flow Rate=0.6 ml/min. LCMS result: retention time=1.80 mins.; observed ion=273.00 (M+H); LCMS Purity=99%.

### Step 2: Preparation of 2-Amino-6-(3,3,3-trifluoropropoxy)nicotinic acid

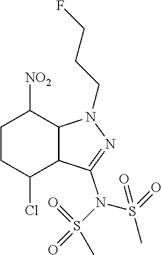 [SMILES: CS(=O)(=O)N(C1=NN(CCCF)C2C1C(Cl)CCC2[N+](=O)[O-])S(C)(=O)=O]

To a stirred solution of 4-chloro-1-(3-fluoropropyl)-7-nitro-1H-indazol-3-amine (6 g, 22.01 mmol) in DCM (200 mL) at 0° C. were added triethylamine (9.20 mL, 66.0 mmol) followed by methanesulfonyl chloride (4.29 mL, 55.0 mmol). The reaction mass was allowed to warm to 27° C. and was stirred for 2 hrs. The progress of the reaction was monitored by TLC (SiO₂, 50% EtOAc/Pet. Rf=0.8). On completion, the reaction mixture was diluted with water (100 mL) and extracted with DCM (2×200 mL). The combined organics were washed with water (100 mL) and then brine (100 mL). The organic layer was dried over anhydrous Na₂SO₄, filtered, and the filtrate was concentrated under reduced pressure to afford N-(4-chloro-1-(3-fluoropropyl)-7-nitro-1H-indazol-3-yl)-N-(methylsulfonyl)methanesulfonamide as pale yellow solid, 8.4 g (85% Yield). The product was used directly in the next step without further purification. ¹H-NMR (300 MHz, CDCl₃) δ: 8.11 (d, J=8.4 Hz, 1H), 7.35 (d, J=8.4 Hz, 1H), 4.79 (t, J=6.8 Hz, 2H), 4.47 (t, J=5.6 Hz, 1H), 4.31 (t, J=5.6 Hz, 1H), 3.62 (s, 6H), 2.33-2.20 (m, 2H). LCMS Method: Column=Acquity BEH C18 (50 mm×2.1 mm, 1.7 um particles); Mobile Phase=A: 0.05% Formic Acid in water; Mobile B=0.05% Formic Acid in acetonitrile; Gradient profile (time (minutes)/% B): 0/3, 0.4/3, 2.5/98, 3.4/98, 3.5/3, 4/3; Column Temp=35° C.; Flow Rate=0.6 mL/min. LCMS result: retention time=1.96 mins.; observed ion=429.94 (M+H); LCMS Purity=96%.

### Step 3: Preparation of N-(4-chloro-1-(3-fluoropropyl)-7-nitro-1H-indazol-3-yl)methanesulfonamide

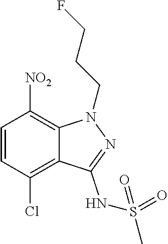 [SMILES: CS(=O)(=O)Nc1nn(CCCF)c2c([N+](=O)[O-])ccc(Cl)c12]

To a stirred solution of N-(4-chloro-1-(3-fluoropropyl)-7-nitro-1H-indazol-3-yl)-N-(methylsulfonyl)methanesulfonamide (8 g, 18.66 mmol) in Ethanol (50 mL) at 27° C. was added NaOH solution (5% in water, 22 mL, 18.66 mmol). The reaction mass was stirred at 27° C. for 1 hr. The progress of the reaction was monitored by TLC (SiO₂, 50% EtOAc/Pet. Rf=0.3). On completion, the reaction mass was cooled to below 10° C. and the pH was adjusted to 2-3 pH via addition of 2 N HCl (˜20 mL). The resulting precipitate was collected via filtration and was washed with water (20 mL), then n-hexane (50 mL), and dried to afford N-(4-chloro-1-(3-fluoropropyl)-7-nitro-1H-indazol-3-yl)methanesulfonamide as yellow solid, 6 g (88% Yield). The product was used directly in the next step without further purification. ¹H-NMR (400 MHz, DMSO-d₆) δ: 8.07 (d, J=8.4 Hz, 1H), 7.35 (s, 1H), 7.19 (d, J=8.4 Hz, 1H), 4.66 (t, J=6.8 Hz, 2H), 4.52 (t, J=5.6 Hz, 1H), 4.40 (t, J=5.6 Hz, 1H), 3.46 (s, 3H), 2.30-2.20 (m, 2H). LCMS Method: Column=Acquity BEH C18 (50 mm×2.1 mm, 1.7 um particles); Mobile Phase=A: 0.05% Formic Acid in water; Mobile B=0.05% Formic Acid in acetonitrile; Gradient profile (time (minutes)/% B): 0/3, 0.4/3, 2.5/98, 3.4/98, 3.5/3, 4/3; Column Temp=35° C.; Flow Rate=0.6 mL/min. LCMS result: retention time=2.16 mins.; observed ion=351.18 (M+H); LCMS Purity=96%.

### Step 4: Preparation of N-(4-chloro-1-(3-fluoropropyl)-7-nitro-1H-indazol-3-yl)-N-(4-methoxybenzyl) methanesulfonamide

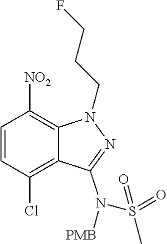

To a stirred solution of N-(4-chloro-1-(3-fluoropropyl)-7-nitro-1H-indazol-3-yl)methanesulfonamide (6 g, 17.11 mmol) in DMF (60 mL) at 27° C. were added K₂CO₃ (7.09 g, 51.3 mmol) followed by 1-(chloromethyl)-4-methoxybenzene (2.77 mL, 20.53 mmol). The reaction mass was stirred at 90° C. for 5 hrs. The progress of the reaction was monitored by TLC (SiO₂, 40% EtOAc/Pet. Rf=0.5). On completion, the reaction mass was quenched with cold water (200 mL) and extracted with EtOAc (2×250 mL). The combined organics were washed with water (100 mL) and then brine (100 mL). The organic layer was dried over anhydrous Na₂SO₄, filtered, and the filtrate was concentrated under reduced pressure. The resulting residue (20 g) was subjected to silica gel chromatography eluting with 10-40% EtOAc/Pet to afford N-(4-chloro-1-(3-fluoropropyl)-7-nitro-1H-indazol-3-yl)-N-(4-methoxybenzyl) methanesulfonamide as pale yellow gum, 6.4 g (74% Yield). ¹H-NMR (400 MHz, CDCl₃) δ: 8.02 (d, J=8.0 Hz, 1H), 7.24-7.21 (m, 3H), 6.79 (d, J=8.8 Hz, 2H), 4.98-4.94 (m, 1H), 4.84-4.79 (m, 1H), 4.71-4.66 (m, 2H), 4.40-4.32 (m, 1H), 4.29-4.21 (m, 1H), 3.76 (s, 3H), 3.03 (s, 3H), 2.23-2.10 (m, 2H). LCMS Method: Column=Acquity BEH C18 (50 mm×2.1 mm, 1.7 um particles); Mobile Phase=A: 0.05% Formic Acid in water; Mobile B=0.05% Formic Acid in acetonitrile; Gradient profile (time (minutes)/% B): 0/3, 0.4/3, 2.5/98, 3.4/98, 3.5/3, 4/3; Column Temp=35° C.; Flow Rate=0.6 mL/min. LCMS result: retention time=2.14 mins.; observed ion=470.93 (M+H); LCMS Purity=93%.

### Step 5: Preparation of N-(7-amino-4-chloro-1-(3-fluoropropyl)-1H-indazol-3-yl)-N-(4-methoxybenzyl)methanesulfonamide

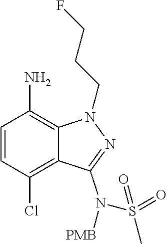

To a stirred suspension of zinc (10.66 g, 163 mmol) in THF (80 mL) at 0° C. were added ammonium chloride (8.72 g, 163 mmol) and water (60 mL) followed by N-(4-chloro-1-(3-fluoropropyl)-7-nitro-1H-indazol-3-yl)-N-(4-methoxybenzyl)methanesulfonamide (6.4 g, 13.59 mmol). The resulting reaction mixture was allowed to warm to 27° C. and was stirred for 3 hrs. The progress of the reaction was monitored by TLC (SiO₂, 60% EtOAc/Pet. Rf=0.6). On completion, the reaction mixture was filtered through a pad of Celite under suction. The filter pad was extracted with EtOAc (250 mL). The combined filtrates were partitioned and the organic layer was reserved while the aqueous layer was back-extracted with EtOAc (2×200 mL). The combined organics (app 750 mL) were washed with water (100 mL) and then brine (100 mL); dried over anhydrous Na₂SO₄; filtered; and the filtrate was concentrated under reduced pressure. The resulting residue (5.5 g) was triturated with n-pentane (3×50 mL) and stirred for 20 minute at 27° C. The solids were isolated by filtered and then were washed with n-pentane (50 mL). Residual volatiles were removed under high vacuum to afford N-(7-amino-4-chloro-1-(3-fluoropropyl)-1H-indazol-3-yl)-N-(4-methoxybenzyl)methanesulfonamide as an off-white solid, 4.93 g (80% Yield). ¹H-NMR (400 MHz, DMSO-d₆) δ: ¹H-NMR (400 MHz, DMSO-d₆) δ: 7.19 (d, J=8.8 Hz, 2H), 6.87 (d, J=7.6 Hz, 1H), 6.79 (d, J=8.8 Hz, 2H), 6.57 (d, J=7.9 Hz, 1H), 5.34 (s, 2H), 4.82-4.60 (m, 4H), 4.29-4.14 (m, 2H), 3.67 (s, 3H), 3.10 (s, 3H), 2.12-2.02 (m, 2H). LCMS Method: Column=Acquity BEH C18 (50 mm×2.1 mm, 1.7 um particles); Mobile Phase=A: 0.05% Formic Acid in water; Mobile B=0.05% Formic Acid in acetonitrile; Gradient profile (time (minutes)/% B): 0/3, 0.4/3, 3.2/98, 3.8/98, 4.2/3, 4.5/3; Column Temp=35° C.; Flow Rate=0.6 mL/min. LCMS result: retention time=2.27 mins.; observed ion=440.95 (M+H); LCMS Purity=97%; HPLC Purity: 98%.

### Preparation of give tert-butyl (S)-(1-(7-bromo-3-(4-chloro-1-(3-fluoropropyl)-3-(N-(4-methoxybenzyl)methylsulfonamido)-1H-indazol-7-yl)-4-oxo-3,4-dihydroquinazolin-2-yl)-2-(3,5-difluorophenyl)ethyl)carbamate

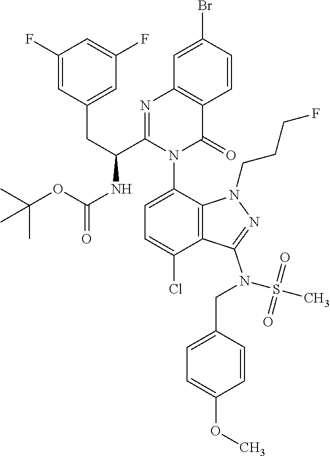 [SMILES: COc1ccc(CN(c2nn(CCCF)c3c(-n4c([C@H](Cc5cc(F)cc(F)c5)NC(=O)OC(C)(C)C)nc5cc(Br)ccc5c4=O)ccc(Cl)c23)S(C)(=O)=O)cc1]

To a solution of (S)-2-((tert-butoxycarbonyl)amino)-3-(3,5-difluorophenyl)propanoic acid (0.752 g, 2.495 mmol) and 2-amino-4-bromobenzoic acid (0.539 g, 2.495 mmol) in acetonitrile (20 mL) at −25° C. was added pyridine (1.468 mL, 18.14 mmol) followed by 2,4,6-tripropyl-1,3,5,2,4,6-trioxatriphosphinane 2,4,6-trioxide (“T3P”, 50% wt. in EtOAc, 6.75 mL, 11.34 mmol). The reaction mixture was stirred as it warmed from −25° C. to 12° C. over 3 h. To the mixture was added N-(7-amino-4-chloro-1-(3-fluoropropyl)-1H-indazol-3-yl)-N-(4-methoxybenzyl)methanesulfonamide (1 g, 2.268 mmol), then the mixture was stirred for 18 h while warming to rt. The reaction mixture was diluted with ethyl acetate; washed with 1N NaOH; then water; then 0.5 M citric acid; then water. The organic phase was dried over Na₂SO₄; filtered; and the filtrate was concentrated under reduced pressure. The resulting residue was purified by silica gel chromatography (80 g RediSep Gold column) eluting with 5-80% ethyl acetate in hexanes to afford tert-butyl (S)-(1-(7-bromo-3-(4-chloro-1-(3-fluoropropyl)-3-(N-(4-methoxybenzyl)methylsulfonamido)-1H-indazol-7-yl)-4-oxo-3,4-dihydroquinazolin-2-yl)-2-(3,5-difluorophenyl)ethyl)carbamate as a light yellow solid, 814 mg (40% yield). LCMS Method: Column=Acquity UPLC BEH C18, 2.1×100 mm, 1.7 μm particles; Solvent A=0.1% Formic acid in 95:5 Water:MeCN; Solvent B=0.1% Formic Acid in 5:95 Water:MeCN; Flow Rate=0.8 mL/min; Gradient Profile (time/% B)=0 min/0%, 3.5 min/100%, 4.5 min/100%.; Detection wavelength=220 and 254 nm. LCMS result: retention time=3.88 min; m/z=846.90 [M-tBu]⁺.

### Preparation of (S)—N-((6P)-7-(2-(1-amino-2-(3,5-difluorophenyl)ethyl)-7-bromo-4-oxoquinazolin-3(4H)-yl)-4-chloro-1-(3-fluoropropyl)-1H-indazol-3-yl)methanesulfonamide

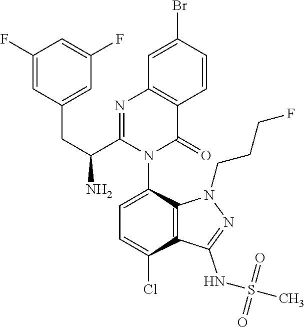

To a solution of tert-butyl (S)-(1-(7-bromo-3-(4-chloro-1-(3-fluoropropyl)-3-(N-(4-methoxybenzyl)methylsulfonamido)-1H-indazol-7-yl)-4-oxo-3,4-dihydroquinazolin-2-yl)-2-(3,5-difluorophenyl)ethyl)carbamate (814 mg, 0.900 mmol) in dichloromethane (DCM) (5 mL) was added TFA (1.387 mL, 18.00 mmol) followed by triflic acid (0.160 mL, 1.800 mmol) and the resulting mixture was stirred at room temp for 1 h. The mixture was concentrated under reduced pressure and the resulting residue was dissolved in ethyl acetate. Residual acid was neutralized by the addition of aq. 1N NaOH. The isolated organic phase was dried over Na₂SO₄; filtered; and the filtrate was concentrated under reduced pressure. The resulting residue was subjected to C18 reverse-phase chromatography (150 g column) eluting with 10-60% Mobile Phase B in Mobile Phase A over 20 minutes (Mobile Phase A=5:95 acetonitrile:water with 0.1% Formic acid; Mobile Phase B: 95:5 acetonitrile:water with 0.1% Formic acid). This chromatography step separates two diastereomers (atropisomers) which correspond to the desired product mass. Fractions corresponding to the second-eluting (major) atropisomer were combined and concentrated under reduced pressure to remove acetonitrile and afford an aqueous mixture. The pH of the aqueous mixture was adjusted to pH=8 by the addition of aq. 1N NaOH. The mixture was extracted with ethyl acetate. The organic phase was washed with brine; dried (Na₂SO₄); filtered; and the filtrate was concentrated under reduced pressure to (S)—N-((6P)-7-(2-(1-amino-2-(3,5-difluorophenyl)ethyl)-7-bromo-4-oxoquinazolin-3(4H)-yl)-4-chloro-1-(3-fluoropropyl)-1H-indazol-3-yl)methanesulfonamide as a white solid, 245 mg (40% yield). 1H NMR (500 MHz, DMSO-d6) δ ppm 8.09 (d, J=8.34 Hz, 1H), 8.05 (d, J=1.79 Hz, 1H), 7.79 (dd, J=8.49, 1.94 Hz, 1H), 7.33-7.43 (m, 2H), 6.96-7.05 (m, 1H), 6.71 (dd, J=8.34, 2.09 Hz, 2H), 4.23-4.34 (m, 1H), 4.13-4.23 (m, 1H), 4.05-4.11 (m, 1H), 3.91 (ddd, J=14.31, 8.34, 5.96 Hz, 1H), 3.51 (dd, J=8.49, 4.62 Hz, 1H), 3.23-3.27 (m, 1H), 3.21 (s, 3H), 2.78 (dd, J=13.41, 8.64 Hz, 1H), 1.88-2.06 (m, 2H).). LCMS Method: Column=Acquity UPLC BEH C18, 2.1×100 mm, 1.7 μm particles; Solvent A=0.1% Formic acid in 95:5 Water:MeCN; Solvent B=0.1% Formic Acid in 5:95 Water:MeCN; Flow Rate=0.8 mL/min; Gradient Profile (time/% B)=0 min/0%, 3.5 min/100%, 4.5 min/100%.; Detection wavelength=220 and 254 nm. LCMS result: retention time=2.27 min; m/z=684.85 [M+H]⁺.

### Preparation of N—((S)-1-((3P)-7-bromo-3-(4-chloro-1-(3-fluoropropyl)-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

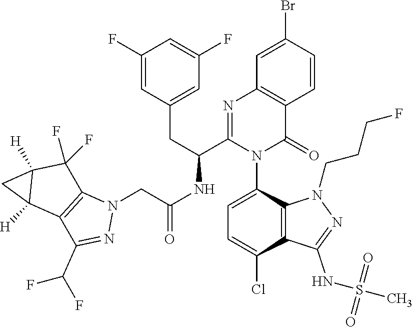

To a solution of (S)—N-((6P)-7-(2-(1-amino-2-(3,5-difluorophenyl)ethyl)-7-bromo-4-oxoquinazolin-3(4H)-yl)-4-chloro-1-(3-fluoropropyl)-1H-indazol-3-yl)methanesulfonamide (240 mg, 0.351 mmol) and 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (93 mg, 0.351 mmol) in ethyl acetate (4 mL) was added 2,6-lutidine (0.102 mL, 0.877 mmol) followed by 2,4,6-tripropyl-1,3,5,2,4,6-trioxatriphosphinane 2,4,6-trioxide (“T3P”, 50% wt. in EtOAc (0.435 mL, 0.702 mmol). The mixture was stirred at room temp for 2 h. The mixture was diluted with water and then extracted with ethyl acetate; dried (Na₂SO₄); filtered; and the filtrate was concentrated under reduced pressure. The resulting residue was triturated with ethyl acetate and the solid were isolated by filtration and then washed with ethyl acetate. Residual solvent was removed under high vac to afford the title compound N—((S)-1-((3P)-7-bromo-3-(4-chloro-1-(3-fluoropropyl)-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide as white solid, 280 mg (86% yield). 1H NMR (500 MHz, DMSO-d6) δ ppm 9.09-9.16 (m, 1H), 8.13 (d, J=8.34 Hz, 1H), 7.97 (d, J=1.79 Hz, 1H), 7.83 (dd, J=8.49, 1.94 Hz, 1H), 7.63-7.74 (m, 1H), 7.36-7.48 (m, 1H), 6.74-7.08 (m, 2H), 6.61 (br d, J=6.56 Hz, 2H), 4.65-4.71 (m, 1H), 4.54-4.60 (m, 1H), 4.46-4.52 (m, 1H), 4.17-4.22 (m, 1H), 4.06-4.15 (m, 1H), 3.80-3.92 (m, 1H), 3.63-3.73 (m, 1H), 3.09-3.17 (m, 2H), 2.94 (dd, J=14.31, 10.73 Hz, 1H), 1.78-1.95 (m, 2H), 1.32-1.38 (m, 1H), 0.83-0.88 (m, 1H). LCMS Method: Column=Acquity UPLC BEH C18, 2.1×100 mm, 1.7 μm particles; Solvent A=0.1% Formic acid in 95:5 Water:MeCN; Solvent B=0.1% Formic Acid in 5:95 Water:MeCN; Flow Rate=0.8 mL/min; Gradient Profile (time/% B)=0 min/0%, 3.5 min/100%, 4.5 min/100%.; Detection wavelength=220 and 254 nm. LCMS result: retention time=3.35 min; m/z=931.2 [M+H]⁺.

### Preparation of tert-Butyl (S)-(1-(7-bromo-3-(4-chloro-1-(2,2-difluoroethyl)-3-(N-(4-methoxybenzyl)methylsulfonamido)-1H-indazol-7-yl)-4-oxo-3,4-dihydroquinazolin-2-yl)-2-(3,5-difluorophenyl)ethyl)carbamate

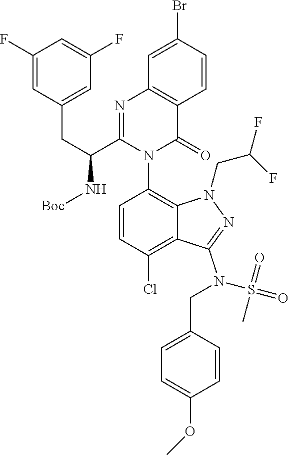 [SMILES: COc1ccc(CN(c2nn(CC(F)F)c3c(-n4c([C@H](Cc5cc(F)cc(F)c5)NC(=O)OC(C)(C)C)nc5cc(Br)ccc5c4=O)ccc(Cl)c23)S(C)(=O)=O)cc1]

To a stirred solution of (S)-2-((tert-butoxycarbonyl)amino)-3-(3,5-difluorophenyl)propanoic acid (15 g, 49.8 mmol) and 2-amino-4-bromobenzoic acid (10.76 g, 49.8 mmol) in pyridine (150 mL) was added diphenyl phosphite (9.64 mL, 49.8 mmol) at 27° C. The mixture was flushed with argon and the flask was then sealed. The reaction mixture was heated to 80° C. and stirred at that temperature for 2 hr. The reaction mixture was cooled to 27° C. and to the mixture was added N-(7-amino-4-chloro-1-(2,2-difluoroethyl)-1H-indazol-3-yl)-N-(4-methoxybenzyl)methanesulfonamide. The flask was sealed and the mixture was heated at 80° C. for 16 hr. The progress of the reaction was monitored by TLC (SiO₂, 30% EtOAc/Pet., Rf=0.4, UV-active). The reaction mixture was allowed to cool to 27° C. and then was concentrated under reduced pressure. The resulting residue was subjected to silca gel column chromatography (Pet.:EtOAc 80:20→470:30) to afford tert-butyl (S)-(1-(7-bromo-3-(4-chloro-1-(2,2-difluoroethyl)-3-(N-(4-methoxybenzyl)methylsulfonamido)-1H-indazol-7-yl)-4-oxo-3,4-dihydroquinazolin-2-yl)-2-(3,5-difluorophenyl)ethyl)carbamate as an off-white solid, 18 g (35%). The isolated material is a mixture of stereoisomers. LCMS: M+H=907.18 and 909.12; purity=89%.

### Preparation of(S)—N-((6P)-7-(2-(1-amino-2-(3,5-difluorophenyl)ethyl)-7-bromo-4-oxoquinazolin-3(4H)-yl)-4-chloro-1-(2,2-difluoroethyl)-1H-indazol-3-yl)methanesulfonamide

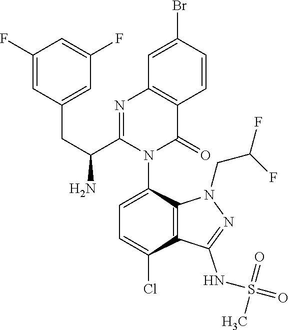

To a stirred solution of tert-butyl (S)-(1-(7-bromo-3-(4-chloro-1-(2,2-difluoroethyl)-3-(N-(4-methoxybenzyl)methylsulfonamido)-1H-indazol-7-yl)-4-oxo-3,4-dihydroquinazolin-2-yl)-2-(3,5-difluorophenyl)ethyl)carbamate (N68085-33-A2, 15 g, 14.70 mmol) in DCM (150 mL) at 27° C. under N2 atmosphere was added TFA (150 mL, 1947 mmol). The solution was stirred for 10 min. To the reaction mixture was added triflic acid (15 mL, 169 mmol). The solution was stirred for 1 h at 27° C. The progress of the reaction was monitored by TLC (SiO₂, 5% MeOH/DCM, Rf=0.4, UV-active). On completion, the solvent was removed under a gentle stream of nitrogen. The residue was dissolved in EtOAc (500 mL), washed with aq saturated NaHCO₃ (2×250 mL), brine (150 mL), dried over Na₂SO₄ and filtered. The filtrate was concentrated under reduced pressure to afford an off-white solid. LCMS analysis of the solid found a 75.42%:21.47% ratio of diastereomers. The crude solid subjected to C18 reverse-phase column chromatography (Mobile Phase: A: 0.1% TFA in water and B: 0.1% TFA in MeCN). Pure fractions containing the major diastereomer (atropisomer) were combined concentrated under reduced pressure. The resulting aqueous solution was made basic via the addition of aq. sat. NaHCO₃; then was extracted with EtOAc (2×500 mL). The combined organic layers were washed with brine (200 mL), dried over Na₂SO₄, filtered and concentrated to afford (S)—N-((6P)-7-(2-(1-amino-2-(3,5-difluorophenyl)ethyl)-7-bromo-4-oxoquinazolin-3(4H)-yl)-4-chloro-1-(2,2-difluoroethyl)-1H-indazol-3-yl)methanesulfonamide as an off-white solid, 8.0 g (76%). LCMS: M+H=687.34, Purity=96%. This material was further purified to isolate the major enantiomer as follows: (S)—N-((6P)-7-(2-(1-amino-2-(3,5-difluorophenyl)ethyl)-7-bromo-4-oxoquinazolin-3(4H)-yl)-4-chloro-1-(2,2-difluoroethyl)-1H-indazol-3-yl)methanesulfonamide (4.5 g, 6.28 mmol) was dissolved in MeOH:MeCN (1:1, 170 mL). The solution was subjected portion-wise to SFC chiral separation as follows: column=(R, R) WHELK-01, 30×250 mm, 5 micron; solvent A=super critical CO₂; solvent B=methanol); eluent composition=50% A:50% B; flow-rate=100 g/min; back-pressure=90 bar; injection volume=1.1 mL; detection=214 nm; Stack time=6.8 min. For each isolated enantiomer, the resulting solution was concentrated under reduced pressure to afford an off-white solid. (S)—N-((6P)-7-(2-(1-amino-2-(3,5-difluorophenyl)ethyl)-7-bromo-4-oxoquinazolin-3(4H)-yl)-4-chloro-1-(2,2-difluoroethyl)-1H-indazol-3-yl)methanesulfonamide as was isolated as the peak eluting from 6 min to 8 min and afforded 2.1 g (48%). ¹H NMR (400 MHz, DMSO-d₆) δ=8.11-8.05 (m, 2H), 7.83-7.78 (m, 1H), 7.47-7.41 (m, 2H), 7.03-6.97 (m, 1H), 6.76-6.69 (m, 2H), 6.41-6.14 (m, 1H), 4.47-4.22 (m, 2H), 3.54-3.49 (m, 1H), 3.25-3.21 (m, 4H), 2.83-2.76 (m, 1H). LCMS: M+H=687.04, Purity=99%, Chiral HPLC Purity=96%.

### Preparation of N—((S)-1-((3P)-7-Bromo-3-(4-chloro-1-(2,2-difluoroethyl)-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

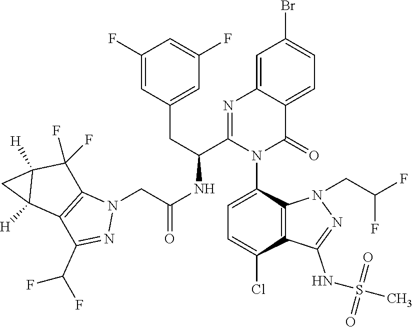

To a solution of (S)—N-((6P)-7-(2-(1-amino-2-(3,5-difluorophenyl)ethyl)-7-bromo-4-oxoquinazolin-3(4H)-yl)-4-chloro-1-(2,2-difluoroethyl)-1H-indazol-3-yl)methanesulfonamide (1.75 g, 2.52 mmol), 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.739 g, 2.77 mmol), HOBt hydrate (0.424 g, 2.77 mmol) and EDC.HCl (0.579 g, 3.02 mmol) in DMF (15 mL) at 27° C. under nitrogen atmosphere was added N-methylmorpholine (2.215 mL, 20.15 mmol). The solution was stirred at 27° C. for 36 h. The progress of the reaction was monitored by TLC (SiO₂, 50% EtOAc/Pet. Rf=0.5, UV-active). The reaction mixture was diluted with ice cold water (50 mL), and stirred for 15 min. The precipitated solid was isolated via filtration, washed with water (50 mL), and dried under vacuum to obtain the crude product. This material was treated with EtOAc (20 mL), stirred for 15 min, and then the solids were isolated via filtration and dried under vacuum to afford N—((S)-1-((3P)-7-bromo-3-(4-chloro-1-(2,2-difluoroethyl)-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide as an off-white solid, 1.6 g (64%). ¹H NMR (400 MHz, DMSO-d₆) δ=10.00 (brs, 1H), 9.23 (d, J=8.1 Hz, 1H), 8.13 (d, J=8.6 Hz, 1H), 7.98 (d, J=2.0 Hz, 1H), 7.85 (dd, J=2.0, 2.1 Hz, 1H), 7.78 (d, J=7.9 Hz, 1H), 7.54 (d, J=7.9 Hz, 1H), 7.07-6.99 (m, 1H), 6.92 (t, J=51.7 Hz, 1H), 6.61 (d, J=6.3 Hz, 2H), 6.11 (t, J=54.6 Hz, 1H), 4.72-4.57 (m, 2H), 4.38 (tt, J=107, 2.9 Hz, 1H), 4.31-4.19 (m, 1H), 3.96-3.83 (m, 1H), 3.44-3.37 (m, 1H), 3.19 (s, 3H), 3.00-2.92 (m, 1H), 2.49-2.45 (m, 2H), 1.39-1.31 (m, 1H), 0.87-0.82 (m, 1H). LCMS: M+H=933.13, LCMS Purity=95%, HPLC Purity=96%, Chiral HPLC Purity=97%.

### Preparation of tert-butyl(S)-(1-(7-bromo-3-(4-chloro-1-(2,2-difluoroethyl)-3-(N-(4-methoxybenzyl) cyclopropanesulfonamido)-1H-indazol-7-yl)-4-oxo-3,4-dihydroquinazolin-2-yl)-2-(3,5-difluorophenyl)ethyl)carbamate

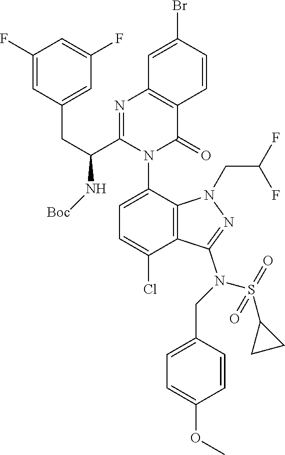 [SMILES: COc1ccc(CN(c2nn(CC(F)F)c3c(-n4c([C@H](Cc5cc(F)cc(F)c5)NC(=O)OC(C)(C)C)nc5cc(Br)ccc5c4=O)ccc(Cl)c23)S(=O)(=O)C2CC2)cc1]

To a stirred solution of (S)-2-((tert-butoxycarbonyl)amino)-3-(3,5-difluorophenyl)propanoic acid (15 g, 49.8 mmol) and 2-amino-4-bromobenzoic acid (12.91 g, 59.7 mmol) in pyridine (150 mL) in a sealed tube at 26° C. was added diphenyl phosphite (35.7 mL, 184 mmol). The reaction mixture was degassed with N2 bubbling for each addition of reagents. The reaction mixture was heated to 80° C. and stirred for 2 hr. The reaction mixture was cooled to 26° C., then N-(7-amino-4-chloro-1-(2,2-difluoroethyl)-1H-indazol-3-yl)-N-(4-methoxybenzyl)cyclopropanesulfonamide (N66734-90-A2, 20.49 g, 34.9 mmol) was added. The mixture was heated at 80° C. for 16 h. The progress of the reaction was monitored by TLC (SiO₂, 30% EtOAc/Pet. Rf=0.3). The reaction mixture was cooled to 26° C. and then was concentrated under reduced pressure. The residue was diluted with water (150 mL) and extracted with ethyl acetate (2×500 mL). The combined organic layers were washed with aq. citric acid (5% w/v, 2×150 mL), then brine (250 mL); dried over anhydrous Na₂SO₄; filtered; and concentrated under reduced pressure to afford a brown gummy liquid (40 g). The above procedure was repeated, and the crude product of both iterations was combined. This material was then subjected to silica gel column chromatography (pet.:EtOAc, 60:40-455:45) to afforded tert-butyl (S)-(1-(7-bromo-3-(4-chloro-1-(2,2-difluoroethyl)-3-(N-(4-methoxybenzyl)cyclopropanesulfonamido)-1H-indazol-7-yl)-4-oxo-3,4-dihydroquinazolin-2-yl)-2-(3,5-difluorophenyl)ethyl)carbamate (mixture of diastereomers) as a yellow solid (42 g, 98%). LCMS: M+H=933.88 & 935.88; purity=76.91%.

### Preparation of(S)—N-((6P)-7-(2-(1-amino-2-(3,5-difluorophenyl)ethyl)-7-bromo-4-oxoquinazolin-3(4H)-yl)-4-chloro-1-(2,2-difluoroethyl)-1H-indazol-3-yl)cyclopropanesulfonamide

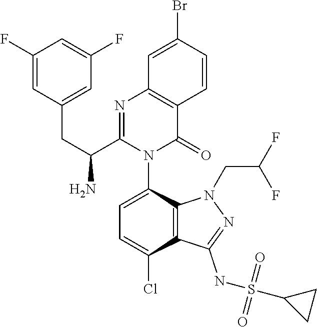

To a stirred solution of tert-butyl (S)-(1-(7-bromo-3-(4-chloro-1-(2,2-difluoroethyl)-3-(N-(4-methoxybenzyl)cyclopropanesulfonamido)-1H-indazol-7-yl)-4-oxo-3,4-dihydroquinazolin-2-yl)-2-(3,5-difluorophenyl)ethyl)carbamate (14 g, 11.53 mmol) in DCM (140 mL) at 27° C. under N2 atmosphere was added TFA (140 mL). The solution was stirred for 10 min. To the solution was added trifluoromethanesulfonic acid (7.16 mL, 81 mmol). The reaction mixture was stirred for 1 h at 27° C. The progress of the reaction was monitored by TLC (SiO₂, 50% EtOAc/pet, Rf=0.2). The solvent was removed under a gentle stream of nitrogen. The residue was dissolved in EtOAc (500 mL) and the organic layer was washed with aq. saturated NaHCO₃ (2×150 mL), brine (50 mL), dried over Na₂SO₄, filtered and concentrated to dryness to the crude compound as an off white solid (12 g). The above procedure was repeated twice more and the additional crude solids (2×14 g) were combined with the above. The combined material was dissolved in dichloromethane (500 mL) and concentrated to afford a homogeneous crude solid. This material was washed with pet. ether:EtOAc (80:20) and then dried under vacuum to afford a brown solid (30 g). This material was then subjected to C18 reverse phase chromatography under the following conditions: Column=RediSep Gold HP C18 275 g; Mobile Phase A=Water:MeCN:TFA (950:50:1); Mobile Phase B=Water:MeCN:TFA (50:950:1); flow rate=80 mL/min; gradient profile (time/% B)=5/5, 5/10, 5/15, 10/20, 15/30, 20/40, 15/45, 10/50; temperature=ambient. Fractions of the major peak were pooled and concentrated under reduced pressure to remove the non-aqueous solvent. The resulting aq. solution was neutralized via the addition of sat. aq. NaHCO₃ (1000 mL), then was extracted with EtOAc (4×500 mL). The combined organics were washed with brine (500 mL), dried over anhydrous Na₂SO₄, filtered and concentrated to afford (S)—N-((6P)-7-(2-(1-amino-2-(3,5-difluorophenyl)ethyl)-7-bromo-4-oxoquinazolin-3(4H)-yl)-4-chloro-1-(2,2-difluoroethyl)-1H-indazol-3-yl)cyclopropanesulfonamide (single diastereomer) as an off white solid. The material was then subjected to SFC purification under the following conditions: Column/dimensions=Chiralpak OX-H (30×250 mm), 5μ; Solvent A=liquid CO₂; Solvent B=Methanol with 0.5% diethyl amine; Eluent=A:B (70:30); Flow-rate=100.0 g/min; Back Pressure=100.0 bar; Detection=UV (214 nm); injection volume=1.3 mL (93 mg/injection); 160 injections. Two peaks were collected separately and the major peak was concentrated under reduced pressure to afford (S)—N-((6P)-7-(2-(1-amino-2-(3,5-difluorophenyl)ethyl)-7-bromo-4-oxoquinazolin-3(4H)-yl)-4-chloro-1-(2,2-difluoroethyl)-1H-indazol-3-yl)cyclopropanesulfonamide (single stereoisomer) as a pale yellow solid, 7.5 g (20%). ¹H NMR (400 MHz, DMSO-d₆) δ=8.11-8.04 (m, 2H), 7.82-7.78 (m, 1H), 7.47-7.39 (m, 2H), 7.02-6.95 (m, 1H), 6.76-6.69 (m, 2H), 6.38-6.19 (m, 1H), 4.48-4.37 (m, 1H), 4.32-4.24 (m, 1H), 3.54-3.48 (m, 1H), 3.3-3.20 (m, 1H), 2.97-2.90 (m, 1H), 2.83-2.76 (m, 1H), 1.05-0.99 (m, 4H). LCMS: M+H=712.94 and 714.94; purity=98.37%, chiral HPLC purity=96%.

### Preparation of N—((S)-1-((3P)-7-bromo-3-(4-chloro-3-(cyclopropanesulfonamido)-1-(2,2-difluoroethyl)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

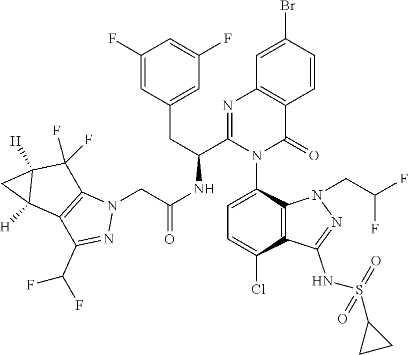

To a stirred solution of (S)—N-((6P)-7-(2-(1-amino-2-(3,5-difluorophenyl)ethyl)-7-bromo-4-oxoquinazolin-3(4H)-yl)-4-chloro-1-(2,2-difluoroethyl)-1H-indazol-3-yl)cyclopropanesulfonamide (500 mg, 0.700 mmol), 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (N68084-15-A1, 185 mg, 0.700 mmol), and HOBt (42.9 mg, 0.280 mmol) in DMF (5 mL) at 27° C. was added N-methylmorpholine (0.308 mL, 2.80 mmol) and N-(3-dimethylaminopropyl)-N′-ethylcarbodiimide hydrochloride (242 mg, 1.261 mmol). The reaction mixture was stirred at 27° C. for 16 h. The progress of the reaction was monitored by TLC (SiO₂, 50% EtOAc/Pet., Rf=0.3, UV-active). On completion, the reaction mixture was diluted with ice cold water (70 mL) and then stirred for 15 min at 27° C. The precipitated solids were collected by filtration and then dried under vacuum to obtain the crude compound as an off-white solid. The crude compound was subjected to silica gel chromatography (pet.:EtOAc (98:2-450:50) to afford N—((S)-1-((3P)-7-bromo-3-(4-chloro-3-(cyclopropanesulfonamido)-1-(2,2-difluoroethyl)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide as an off-white solid, 550 mg (80%). ¹H NMR (400 MHz, DMSO-d₆) δ=9.99 (s, 1H), 9.24 (d, J=8.1 Hz, 1H), 8.13 (d, J=8.8 Hz, 1H), 7.97 (d, J=1.8 Hz, 1H), 7.87-7.83 (m, 1H), 7.77 (d, J=7.9 Hz, 1H), 7.54 (d, J=7.9 Hz, 1H), 7.06-6.79 (m, 2H), 6.64-6.58 (m, 2H), 6.23-5.98 (m, 1H), 4.74-4.57 (m, 2H), 4.41-4.35 (m, 1H), 4.29-4.16 (m, 1H), 3.94-3.84 (m, 1H), 3.38-3.34 (m, 1H), 3.02-2.93 (m, 1H), 2.90-2.83 (m, 1H), 2.48-2.35 (m, 2H), 1.37-1.30 (m, 1H), 1.02-0.90 (m, 4H), 0.87-0.82 (m, 1H). LCMS analysis method F: RT=6.74 mins, (M+H)=959.0 and 961.0; LCMS Purity=98%; Chiral HPLC Purity=98%.

### General Procedure N:

To a stirred solution of (S)—N-((6P)-7-(2-(1-amino-2-(3,5-difluorophenyl)ethyl)-7-(1-(2,2-difluoropropyl)-1H-pyrazol-3-yl)-4-oxoquinazolin-3(4H)-yl)-4-chloro-1-methyl-1H-indazol-3-yl)methanesulfonamide (25 mg, 0.036 mmol) in N,N-Dimethylformamide (DMF) (1 mL) were added the appropriate carboxylic acid, DIPEA (0.019 mL, 0.107 mmol) and 2,4,6-tripropyl-1,3,5,2,4,6-trioxatriphosphinane 2,4,6-trioxide (0.044 mL, 0.071 mmol). The reaction mixture was stirred at room temp for 1.5 h. To the mixture was added ammonia in methanol (2M, 1 mL) and stirring was continued for 1.5 h. The reaction mixture was then filtered and purified by HPLC to afford the title compound.

### Preparation of Example 55: N—((S)-1-((3P)-3-(4-chloro-1-methyl-3-(methylsulfonamido)-1H-indazol-7-yl)-7-(2-(fluoromethyl)pyrimidin-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

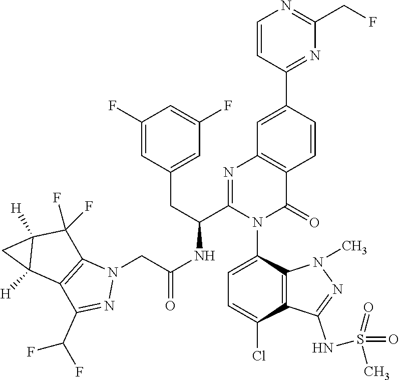

The title compound was prepared according to General Procedure L using 4-chloro-2-(fluoromethyl)pyrimidine as the coupling partner modified as follows: SPhos Pd G3 (0.1 equiv) was used in place of Pd(OAc)₂ and dicyclohexyl(2′,6′-dimethoxy-[1,1′-biphenyl]-2-yl)phosphane, and the reaction was run at 60 deg C. for 24 hr. The experiment afforded the title compound, N—((S)-1-((3P)-3-(4-chloro-1-methyl-3-(methylsulfonamido)-1H-indazol-7-yl)-7-(2-(fluoromethyl)pyrimidin-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.393 min.; observed ion=915.4 (M+H). 1H NMR (500 MHz, CDCl3, 303 K) δ (ppm)=9.01 (d, J=5.1 Hz, 1H), 8.48 (d, J=1.5 Hz, 1H), 8.46 (d, J=8.3 Hz, 1H), 8.31 (dd, J=1.5, 8.3 Hz, 1H), 7.87 (d, J=5.1 Hz, 1H), 7.18 (d, J=8.0 Hz, 1H), 6.87-6.60 (m, 4H), 6.40 (d, J=6.3 Hz, 2H), 5.78-5.64 (m, 2H), 4.82-4.77 (m, 1H), 4.72-4.66 (m, 2H), 3.57 (s, 3H), 3.42 (s, 3H), 3.21 (dd, J=6.0, 13.7 Hz, 1H), 2.90 (dd, J=7.2, 13.7 Hz, 1H), 2.61-2.55 (m, 1H), 2.43 (td, J=4.1, 8.7 Hz, 1H), 1.47-1.42 (m, 1H), 1.34-1.24 (m, 1H), 1.18-1.12 (m, 1H)

### Preparation of Example 56: N—((S)-1-((3P)-3-(4-chloro-1-methyl-3-(methylsulfonamido)-1H-indazol-7-yl)-7-(2-(difluoromethyl)pyrimidin-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

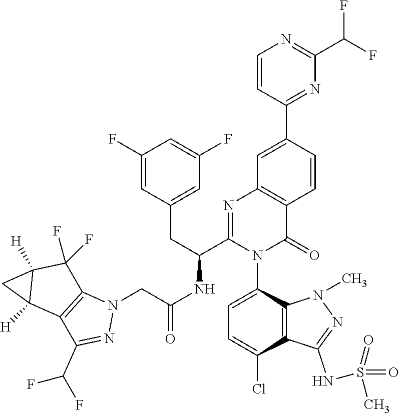

The title compound was prepared according to General Procedure L using 4-chloro-2-(difluoromethyl)pyrimidine as the coupling partner modified as follows: SPhos Pd G3 (0.1 equiv) was used in place of Pd(OAc)₂ and dicyclohexyl(2′,6′-dimethoxy-[1,1′-biphenyl]-2-yl)phosphane, and the reaction was run at 60 deg C. for 24 hr. The experiment afforded the title compound, N—((S)-1-((3P)-3-(4-chloro-1-methyl-3-(methylsulfonamido)-1H-indazol-7-yl)-7-(2-(difluoromethyl)pyrimidin-4-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method C: retention time=3.166 min.; observed ion=933 (M+H). 1H NMR (500 MHz, CD3OD, 303 K) δ (ppm)=9.10 (d, J=5.4 Hz, 1H), 8.79 (s, 1H), 8.50-8.44 (m, 2H), 8.34 (d, J=5.1 Hz, 1H), 7.33 (d, J=8.0 Hz, 1H), 7.25 (d, J=7.7 Hz, 1H), 7.01-6.57 (m, 5H), 4.85-4.82 (m, 1H), 4.58-4.49 (m, 2H), 3.65 (s, 3H), 3.51 (dd, J=4.9, 14.2 Hz, 1H), 3.27 (s, 3H), 3.14 (dd, J=9.5, 14.0 Hz, 1H), 2.48-2.39 (m, 2H), 1.39-1.31 (m, 1H), 1.03-0.98 (m, 1H)

### Preparation of Example 57: N—((S)-1-((3P)-3-(4-chloro-1-methyl-3-(methylsulfonamido)-1H-indazol-7-yl)-7-(5-methyl-1-(2,2,3,3-tetrafluoropropyl)-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

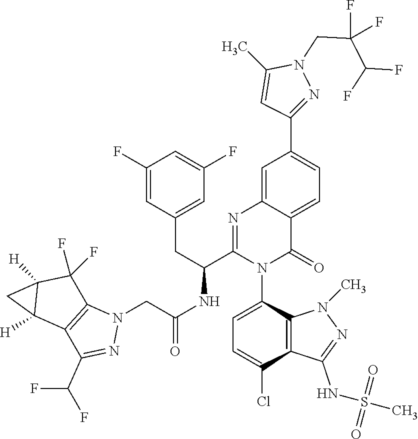

The title compound was prepared according to General Procedure M using 2,2,3,3-tetrafluoropropyl trifluoromethanesulfonate as the coupling partner modified as follows: the reaction temperature was 70 deg C., the reaction time was 2 h, and the pyrazole was N—((S)-1-((3P)-3-(4-chloro-3-(N-(4-methoxybenzyl)methylsulfonamido)-1-methyl-1H-indazol-7-yl)-7-(5-methyl-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The experiment afforded the title compound, N—((S)-1-((3P)-3-(4-chloro-1-methyl-3-(methylsulfonamido)-1H-indazol-7-yl)-7-(5-methyl-1-(2,2,3,3-tetrafluoropropyl)-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=3.75 min.; observed ion=999.05 (M+H). 1H NMR (500 MHz, METHANOL-d4) δ ppm 8.23-8.30 (m, 2H), 8.07 (dd, J=8.34, 1.79 Hz, 1H), 7.26-7.32 (m, 1H), 7.18 (d, J=8.05 Hz, 1H), 6.55-6.81 (m, 5H), 6.17-6.43 (m, 1H), 4.79-4.85 (m, 2H), 4.47-4.55 (m, 2H), 3.61 (s, 3H), 3.46 (dd, J=14.01, 5.07 Hz, 1H), 3.24 (s, 3H), 3.09 (dd, J=14.16, 9.09 Hz, 1H), 2.44 (s, 3H), 2.32-2.42 (m, 2H), 1.31-1.36 (m, 1H), 0.99 (dtd, J=5.74, 3.91, 3.91, 2.24 Hz, 1H).

### Preparation of Example 58: (S)—N—((S)-1-((3P)-3-(4-chloro-1-methyl-3-(methylsulfonamido)-1H-indazol-7-yl)-7-(1-(2,2-difluoropropyl)-1H-pyrazol-3-yl)-4-oxo-3,4-dihydroquinazolin-2-yl)-2-(3,5-difluorophenyl)ethyl)-2-(3-cyclopropyl-1H-pyrazol-1-yl)propanamide

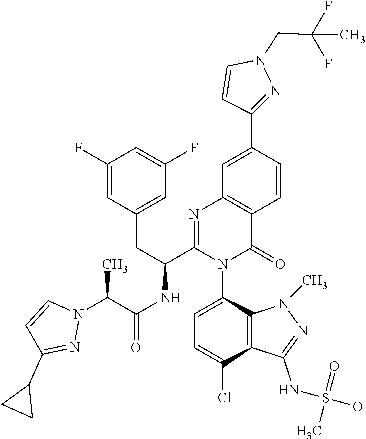

The title compound was prepared according to General Procedure N using (S)-2-(3-cyclopropyl-1H-pyrazol-1-yl)propanoic acid as the coupling partner. The experiment afforded the title compound, (S)—N—((S)-1-((3P)-3-(4-chloro-1-methyl-3-(methylsulfonamido)-1H-indazol-7-yl)-7-(1-(2,2-difluoropropyl)-1H-pyrazol-3-yl)-4-oxo-3,4-dihydroquinazolin-2-yl)-2-(3,5-difluorophenyl)ethyl)-2-(3-cyclopropyl-1H-pyrazol-1-yl)propanamide. The sample was analyzed using LCMS Method H: retention time=1.48 min.; observed ion=865.4 (M+H). 1H NMR (500 MHz, METHANOL-d4) δ ppm 8.24-8.35 (m, 2H), 8.11 (dd, J=8.34, 1.79 Hz, 1H), 7.82 (d, J=2.38 Hz, 1H), 7.37 (d, J=2.38 Hz, 1H), 7.32 (d, J=7.75 Hz, 1H), 7.22-7.28 (m, 1H), 6.96 (d, J=2.38 Hz, 1H), 6.73 (tt, J=9.24, 2.38 Hz, 1H), 6.64 (dd, J=8.34, 2.09 Hz, 2H), 5.93 (d, J=2.38 Hz, 1H), 4.95 (dd, J=9.09, 5.22 Hz, 1H), 4.69 (t, J=12.52 Hz, 2H), 4.51 (q, J=7.15 Hz, 1H), 3.47 (s, 3H), 3.42-3.46 (m, 1H), 3.28 (s, 3H), 3.08 (dd, J=14.01, 8.94 Hz, 1H), 1.87 (tt, J=8.42, 4.99 Hz, 1H), 1.68 (t, J=18.78 Hz, 3H), 1.31 (d, J=7.15 Hz, 3H), 0.84-0.88 (m, 2H), 0.60-0.68 (m, 2H).

### Preparation of Example 59: N—((S)-1-((3P)-3-(4-chloro-1-methyl-3-(methylsulfonamido)-1H-indazol-7-yl)-4-oxo-7-(1-(2,2,3,3-tetrafluoropropyl)-1H-pyrazol-3-yl)-3,4-dihydroquinazolin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide

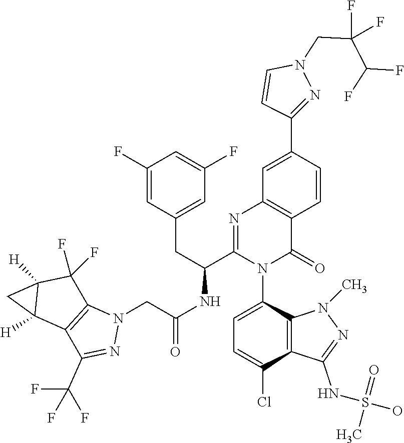

The title compound was prepared according to General Procedure N using 2-((3bS,4a R)-5,5-difluoro-3-(trifluoromethyl)-3b,4,4a,5-tetrahydro-1H-cyclopropa[3,4]cyclopenta[1,2-c]pyrazol-1-yl)acetic acid as the coupling partner, with the following modification: the pyrazole used was (S)—N-((6P)-7-(2-(1-amino-2-(3,5-difluorophenyl)ethyl)-4-oxo-7-(1-(2,2,3,3-tetrafluoropropyl)-1H-pyrazol-3-yl)quinazolin-3(4H)-yl)-4-chloro-1-methyl-1H-indazol-3-yl)methanesulfonamide. The experiment afforded the title compound, N—((S)-1-((3P)-3-(4-chloro-1-methyl-3-(methylsulfonamido)-1H-indazol-7-yl)-4-oxo-7-(1-(2,2,3,3-tetrafluoropropyl)-1H-pyrazol-3-yl)-3,4-dihydroquinazolin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide. The sample was analyzed using LCMS Method B: retention time=3.45 min., observed ion=1003 (M+H). 1H NMR (500 MHz, METHANOL-d4) δ ppm 8.27-8.33 (m, 2H), 8.12 (dd, J=8.35, 1.49 Hz, 1H), 7.87 (d, J=2.38 Hz, 1H), 7.29 (d, J=7.75 Hz, 1H), 7.17 (d, J=7.75 Hz, 1H), 6.98 (d, J=2.38 Hz, 1H), 6.77 (tt, J=9.13, 2.35 Hz, 1H), 6.60 (dd, J=8.05, 2.09 Hz, 2H), 6.12-6.39 (m, 1H), 4.95 (t, J=14.16 Hz, 2H), 4.80-4.84 (m, 1H), 4.52-4.65 (m, 2H), 3.61 (s, 3H), 3.48 (dd, J=14.01, 5.36 Hz, 1H), 3.24 (s, 3H), 3.10 (dd, J=14.01, 9.24 Hz, 1H), 2.40-2.51 (m, 2H), 1.34-1.40 (m, 1H), 1.01-1.08 (m, 1H).

### Preparation of Example 60: N—((S)-1-((3P)-3-(4-chloro-1-methyl-3-(methylsulfonamido)-1H-indazol-7-yl)-7-(5-methyl-1-(2,2,3,3,3-pentafluoropropyl)-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

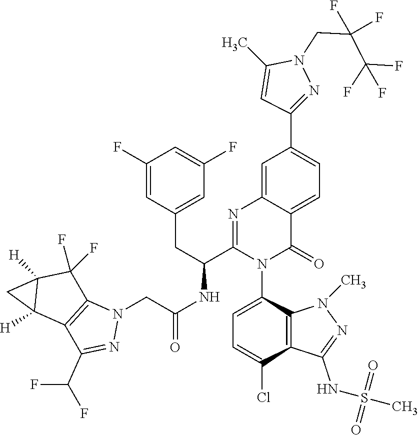

The title compound was prepared according to General Procedure M using 2,2,3,3,3-pentafluoropropyl trifluoromethanesulfonate as the coupling partner, and modified as follows: the pyrazole used was N—((S)-1-((3P)-3-(4-chloro-3-(N-(4-methoxybenzyl)methylsulfonamido)-1-methyl-1H-indazol-7-yl)-7-(5-methyl-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The experiment afforded the title compound, N—((S)-1-((3P)-3-(4-chloro-1-methyl-3-(methylsulfonamido)-1H-indazol-7-yl)-7-(5-methyl-1-(2,2,3,3,3-pentafluoropropyl)-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=3.85 min., observed ion=1017 (M+H). 1H NMR (500 MHz, METHANOL-d4) δ ppm 8.24-8.32 (m, 2H), 8.07 (dd, J=8.20, 1.64 Hz, 1H), 7.29 (d, J=8.05 Hz, 1H), 7.18 (d, J=7.75 Hz, 1H), 6.54-6.80 (m, 5H), 5.05 (t, J=14.60 Hz, 2H), 4.82-4.85 (m, 1H), 4.46-4.56 (m, 2H), 3.61 (s, 3H), 3.47 (dd, J=14.01, 5.07 Hz, 1H), 3.23 (s, 3H), 3.09 (dd, J=14.16, 9.39 Hz, 1H), 2.45 (s, 3H), 2.36-2.43 (m, 2H), 1.31-1.37 (m, 1H), 0.96-1.02 (m, 1H).

### Preparation of Example 61: (S)—N-(1-((3P)-3-(4-chloro-1-methyl-3-(methylsulfonamido)-1H-indazol-7-yl)-7-(1-(2,2-difluoropropyl)-1H-pyrazol-3-yl)-4-oxo-3,4-dihydroquinazolin-2-yl)-2-(3,5-difluorophenyl)ethyl)-2-(3-cyclopropyl-1H-pyrazol-1-yl)acetamide

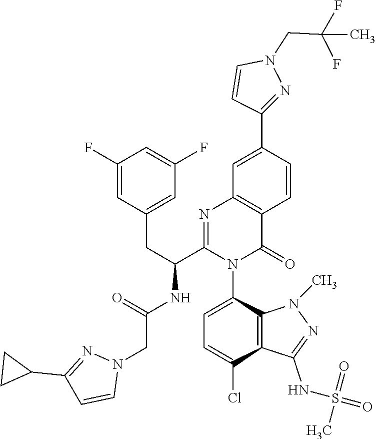

The title compound was prepared according to General Procedure N using 2-(3-cyclopropyl-1H-pyrazol-1-yl)acetic acid as the coupling partner. The experiment afforded the title compound, (S)—N-(1-((3P)-3-(4-chloro-1-methyl-3-(methylsulfonamido)-1H-indazol-7-yl)-7-(1-(2,2-difluoropropyl)-1H-pyrazol-3-yl)-4-oxo-3,4-dihydroquinazolin-2-yl)-2-(3,5-difluorophenyl)ethyl)-2-(3-cyclopropyl-1H-pyrazol-1-yl)acetamide. The sample was analyzed using LCMS Method H: retention time=1.42 min.; observed ion=851.4 (M+H). 1H NMR (500 MHz, METHANOL-d4) δ ppm 8.24-8.34 (m, 2H), 8.11 (dd, J=8.34, 1.49 Hz, 1H), 7.82 (d, J=2.38 Hz, 1H), 7.27-7.33 (m, 2H), 7.20 (d, J=7.75 Hz, 1H), 6.96 (d, J=2.38 Hz, 1H), 6.74 (tt, J=9.20, 2.27 Hz, 1H), 6.66 (dd, J=8.20, 2.24 Hz, 2H), 5.92 (d, J=2.38 Hz, 1H), 4.93-4.96 (m, 1H), 4.69 (t, J=12.52 Hz, 2H), 4.21-4.42 (m, 2H), 3.60 (s, 3H), 3.49 (dd, J=14.01, 5.66 Hz, 1H), 3.26 (s, 3H), 3.07 (dd, J=14.01, 8.64 Hz, 1H), 1.81-1.88 (m, 1H), 1.68 (t, J=18.63 Hz, 3H), 0.82-0.88 (m, 2H), 0.59-0.67 (m, 2H).

### Preparation of 1-(2,2,3,3-tetrafluoropropyl)-3-(4,4,5,5-tetramethyl-1,3,2-dioxaborolan-2-yl)-1H-pyrazole and 1-(2,2,3,3-tetrafluoropropyl)-5-(4,4,5,5-tetramethyl-1,3,2-dioxaborolan-2-yl)-1H-pyrazole

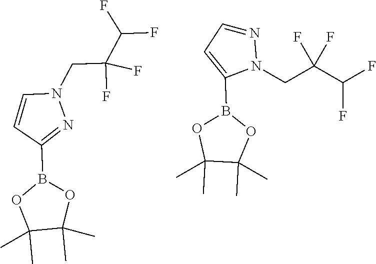 [SMILES: CC1(C)OB(c2ccn(CC(F)(F)C(F)F)n2)OC1(C)C ; CC1(C)OB(c2ccnn2CC(F)(F)C(F)F)OC1(C)C]

To a mixture of 3-(4,4,5,5-tetramethyl-1,3,2-dioxaborolan-2-yl)-1H-pyrazole (100 mg, 0.515 mmol) and 2,2,3,3-tetrafluoropropyl trifluoromethanesulfonate (272 mg, 1.031 mmol) in acetonitrile (2 mL) was added cesium carbonate (252 mg, 0.773 mmol) and the resulting mixture was heated at 70° C. for 2 h. After cooling to room temperature, water was added and the mixture was extracted with ethyl acetate, dried (Na₂SO₄), filtered and the filtrate was concentrated under reduced pressure to afford the title compound as a mixture of two isomers. The product mixture was used in subsequent steps without further purification. LC/MS m/z=226.90 [M+H]⁺.

### Preparation of Example 62: N—((S)-1-((3P)-3-(4-chloro-1-(3-fluoropropyl)-3-(methylsulfonamido)-1H-indazol-7-yl)-4-oxo-7-(1-(2,2,3,3-tetrafluoropropyl)-1H-pyrazol-5-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide and

### Preparation of Example 67: N—((S)-1-((3P)-3-(4-chloro-1-(3-fluoropropyl)-3-(methylsulfonamido)-1H-indazol-7-yl)-4-oxo-7-(1-(2,2,3,3-tetrafluoropropyl)-1H-pyrazol-3-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

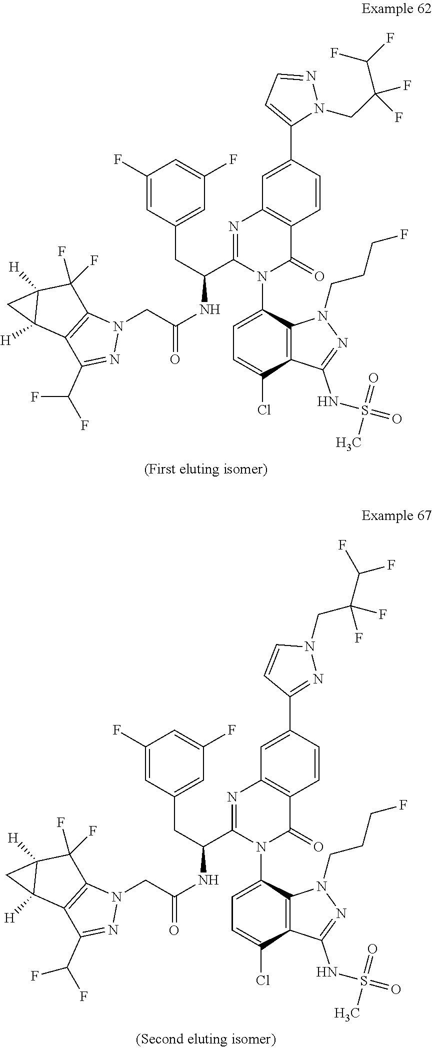

To a solution of N—((S)-1-((3P)-7-bromo-3-(4-chloro-1-(3-fluoropropyl)-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (30 mg, 0.032 mmol), [1-(2,2,3,3-tetrafluoropropyl)-3-(4,4,5,5-tetramethyl-1,3,2-dioxaborolan-2-yl)-1H-pyrazole and 1-(2,2,3,3-tetrafluoropropyl)-5-(4,4,5,5-tetramethyl-1,3,2-dioxaborolan-2-yl)-1H-pyrazole mixture, see above preparation] (29.8 mg, 0.097 mmol) and K₃PO₄ (20.54 mg, 0.097 mmol) in Tetrahydrofuran (THF) (1 mL)/Water (0.250 mL) was added Dichloro[9,9-dimethyl-4,5-bis(diphenylphosphino)xanthene]palladium(II) (2.438 mg, 3.23 μmol) and the resulting mixture was heated at 50° C. for 2 h. The mixture was then cooled to room temp; diluted with ethyl acetate; washed with water; dried (Na₂SO₄); filtered; and the filtrate was concentrated under reduced pressure. The resulting residue was subjected to HPLC purification (Column=two identical columns connected in series, Sunfire C18 OBD, 30×100 mm, 5 μm particles; Solvent A=water:MeCN 95:5 w/ 0.1% Formic Acid, Solvent B=MeCN:water 95:5 w/ 0.1% Formic Acid; Flow Rate=42 mL/min; Gradient profile (time (minutes)/% Solvent B in Solvent A)=0/30, 15/100, 20/100; Detection wavelength=220 and 254 nm.). Two isomers corresponding to the desired product mass were isolated:

### First Isomer to Elute:

Example 62: The sample was analyzed using LCMS Method B: retention time=3.22 min.; observed ion=1031.05 (M+H). 1H NMR (500 MHz, METHANOL-d4) δ ppm 8.37 (d, J=8.05 Hz, 1H), 7.96-8.02 (m, 1H), 7.68-7.77 (m, 2H), 7.34 (d, J=7.75 Hz, 1H), 7.26 (d, J=8.05 Hz, 1H), 6.51-6.80 (m, 5H), 6.04-6.31 (m, 1H), 4.92 (s, 1H), 4.80-4.84 (m, 1H), 4.50-4.62 (m, 2H), 4.06-4.29 (m, 3H), 3.78 (ddd, J=14.23, 8.27, 6.11 Hz, 1H), 3.42 (dd, J=14.01, 5.07 Hz, 1H), 3.25 (s, 3H), 3.05 (dd, J=14.01, 9.24 Hz, 1H), 2.41 (td, J=7.67, 3.73 Hz, 2H), 1.94-2.00 (m, 1H), 1.31-1.37 (m, 1H), 0.96-1.02 (m, 1H).

### Second Isomer to Elute:

Example 67: The sample was analyzed using LCMS Method B: retention time=3.26 min.; observed ion=1031 (M+H). 1H NMR (500 MHz, METHANOL-d4) δ ppm 8.31 (d, J=1.49 Hz, 1H), 8.28 (d, J=8.34 Hz, 1H), 8.11 (dd, J=8.34, 1.49 Hz, 1H), 7.88 (d, J=2.38 Hz, 1H), 7.32 (d, J=7.75 Hz, 1H), 7.20 (d, J=7.75 Hz, 1H), 6.98 (d, J=2.38 Hz, 1H), 6.53-6.80 (m, 4H), 6.14-6.40 (m, 1H), 4.95 (t, J=14.16 Hz, 2H), 4.82 (dd, J=9.09, 4.92 Hz, 1H), 4.53-4.64 (m, 2H), 4.06-4.29 (m, 3H), 3.78 (ddd, J=14.08, 8.12, 6.11 Hz, 1H), 3.41 (dd, J=13.71, 5.07 Hz, 1H), 3.25 (s, 3H), 3.05 (dd, J=13.86, 9.09 Hz, 1H), 2.37-2.46 (m, 2H), 1.91-2.04 (m, 2H), 1.31-1.38 (m, 1H), 0.98-1.03 (m, 1H).

### Preparation of Example 63: (S)—N-(1-((3P)-3-(4-chloro-1-methyl-3-(methylsulfonamido)-1H-indazol-7-yl)-4-oxo-7-(1-(2,2,3,3-tetrafluoropropyl)-1H-pyrazol-3-yl)-3,4-dihydroquinazolin-2-yl)-2-(3,5-difluorophenyl)ethyl)-2-(3-cyclopropyl-1H-pyrazol-1-yl)acetamide

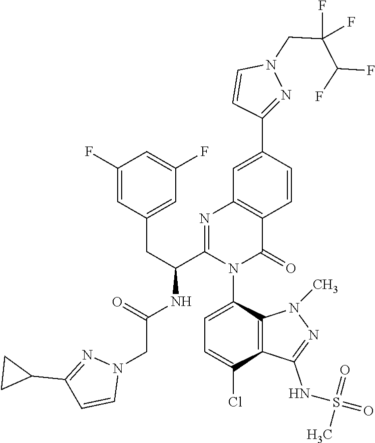

The title compound was prepared according to General Procedure N using 2-(3-cyclopropyl-1H-pyrazol-1-yl)acetic acid as the coupling partner, with the following modification: the pyrazole used was (S)—N-((6P)-7-(2-(1-amino-2-(3,5-difluorophenyl)ethyl)-4-oxo-7-(1-(2,2,3,3-tetrafluoropropyl)-1H-pyrazol-3-yl)quinazolin-3(4H)-yl)-4-chloro-1-methyl-1H-indazol-3-yl)methanesulfonamide. The experiment afforded the title compound, (S)—N-(1-((3P)-3-(4-chloro-1-methyl-3-(methylsulfonamido)-1H-indazol-7-yl)-4-oxo-7-(1-(2,2,3,3-tetrafluoropropyl)-1H-pyrazol-3-yl)-3,4-dihydroquinazolin-2-yl)-2-(3,5-difluorophenyl)ethyl)-2-(3-cyclopropyl-1H-pyrazol-1-yl)acetamide. The sample was analyzed using LCMS Method H: retention time=1.43 min.; observed ion=887.4 (M+H). 1H NMR (500 MHz, METHANOL-d4) δ ppm 8.27-8.35 (m, 2H), 8.12 (dd, J=8.35, 1.79 Hz, 1H), 7.87 (d, J=2.68 Hz, 1H), 7.32 (d, J=2.38 Hz, 1H), 7.29 (d, J=7.75 Hz, 1H), 7.20 (d, J=7.75 Hz, 1H), 6.99 (d, J=2.38 Hz, 1H), 6.74 (tt, J=9.20, 2.27 Hz, 1H), 6.59-6.68 (m, 2H), 6.11-6.40 (m, 1H), 5.92 (d, J=2.09 Hz, 1H), 4.91-5.00 (m, 3H), 4.35-4.43 (m, 1H), 4.25-4.34 (m, 1H), 3.59 (s, 3H), 3.49 (dd, J=13.86, 5.81 Hz, 1H), 3.26 (s, 3H), 3.07 (dd, J=13.71, 8.64 Hz, 1H), 1.81-1.87 (m, 1H), 0.83-0.89 (m, 2H), 0.58-0.66 (m, 2H).

### Preparation of Example 64: N—((S)-1-((3P)-3-(4-chloro-1-(2,2-difluoroethyl)-3-(methylsulfonamido)-1H-indazol-7-yl)-7-(1-(2,2-difluoropropyl)-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

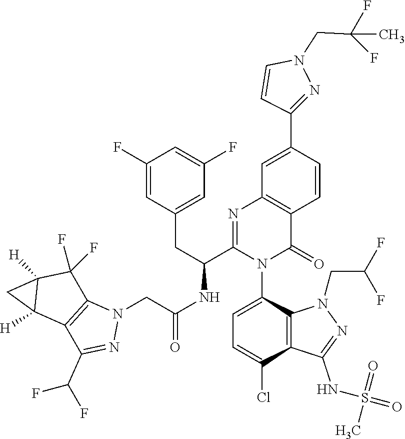

To a mixture of N—((S)-1-((3P)-7-bromo-3-(4-chloro-1-(2,2-difluoroethyl)-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (30 mg, 0.032 mmol), 1-(2,2-difluoropropyl)-3-(tributylstannyl)-1H-pyrazole (20.97 mg, 0.048 mmol) and copper(I) iodide (0.612 mg, 3.21 μmol) in N,N-Dimethylformamide (DMF) (1.5 mL) was added tetrakis(triphenylphosphine)palladium(0) (3.71 mg, 3.21 μmol). The mixture was degassed (brief high vacuum, then refilled with Ar) and then heated at 100° C. for 3 h. The mixture was cooled to room temperature; filtered; and the filtrate was directly subjected to HPLC purification to afford the title compound, N—((S)-1-((3P)-3-(4-chloro-1-(2,2-difluoroethyl)-3-(methylsulfonamido)-1H-indazol-7-yl)-7-(1-(2,2-difluoropropyl)-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.48 min.; observed ion=999.4 (M+H). 1H NMR (500 MHz, METHANOL-d4) δ ppm 8.29 (dd, J=9.69, 1.34 Hz, 2H), 8.11 (dd, J=8.35, 1.79 Hz, 1H), 7.83 (d, J=2.38 Hz, 1H), 7.36 (d, J=7.75 Hz, 1H), 7.24 (d, J=7.75 Hz, 1H), 6.96 (d, J=2.68 Hz, 1H), 6.51-6.81 (m, 4H), 5.87-6.14 (m, 1H), 4.74 (dd, J=9.24, 4.77 Hz, 1H), 4.64-4.71 (m, 2H), 4.53-4.62 (m, 2H), 4.30-4.42 (m, 1H), 3.89-4.01 (m, 1H), 3.41 (dd, J=14.16, 4.92 Hz, 1H), 3.24 (s, 3H), 3.06 (dd, J=14.01, 9.24 Hz, 1H), 2.34-2.45 (m, 2H), 1.68 (t, J=18.78 Hz, 3H), 1.31-1.37 (m, 1H), 0.93-1.02 (m, 1H).

### Preparation of Example 65: (R)—N—((S)-1-((3P)-3-(4-chloro-1-methyl-3-(methylsulfonamido)-1H-indazol-7-yl)-7-(1-(2,2-difluoropropyl)-1H-pyrazol-3-yl)-4-oxo-3,4-dihydroquinazolin-2-yl)-2-(3,5-difluorophenyl)ethyl)-2-(3-cyclopropyl-1H-pyrazol-1-yl)propanamide

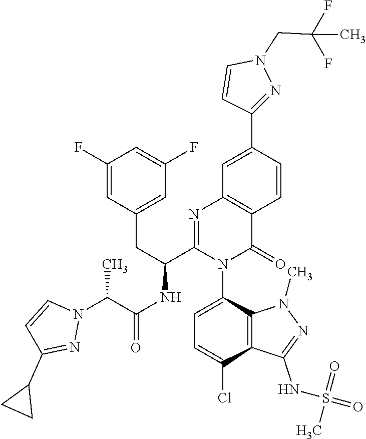

The title compound was prepared according to General Procedure N using (R)-2-(3-cyclopropyl-1H-pyrazol-1-yl)propanoic acid as the coupling partner. The experiment afforded the title compound, (R)—N—((S)-1-((3P)-3-(4-chloro-1-methyl-3-(methylsulfonamido)-1H-indazol-7-yl)-7-(1-(2,2-difluoropropyl)-1H-pyrazol-3-yl)-4-oxo-3,4-dihydroquinazolin-2-yl)-2-(3,5-difluorophenyl)ethyl)-2-(3-cyclopropyl-1H-pyrazol-1-yl)propanamide. The sample was analyzed using LCMS Method H: retention time=1.46 min.; observed ion=865.4 (M+H). 1H NMR (500 MHz, METHANOL-d4) δ ppm 8.26-8.33 (m, 2H), 8.10 (dd, J=8.34, 1.49 Hz, 1H), 7.83 (d, J=2.38 Hz, 1H), 7.40 (d, J=2.38 Hz, 1H), 7.32 (d, J=8.05 Hz, 1H), 7.20 (d, J=7.75 Hz, 1H), 6.95 (d, J=2.38 Hz, 1H), 6.78 (tt, J=9.16, 2.31 Hz, 1H), 6.54 (dd, J=8.05, 2.09 Hz, 2H), 5.95 (d, J=2.38 Hz, 1H), 4.81 (dd, J=9.39, 4.92 Hz, 1H), 4.64-4.77 (m, 3H), 3.48 (s, 3H), 3.38 (dd, J=13.86, 4.92 Hz, 1H), 3.26 (s, 3H), 3.05 (dd, J=14.01, 9.54 Hz, 1H), 1.83 (tt, J=8.49, 5.07 Hz, 1H), 1.68 (t, J=18.78 Hz, 3H), 1.40 (d, J=7.15 Hz, 3H), 0.82 (ddt, J=8.34, 2.31, 1.38, 1.38 Hz, 2H), 0.55-0.67 (m, 2H).

### Preparation of Example 66: N—((S)-1-((3P)-3-(4-chloro-1-methyl-3-(methylsulfonamido)-1H-indazol-7-yl)-7-(1-(2,2-difluoropropyl)-1H-pyrazol-3-yl)-4-oxo-3,4-dihydroquinazolin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide

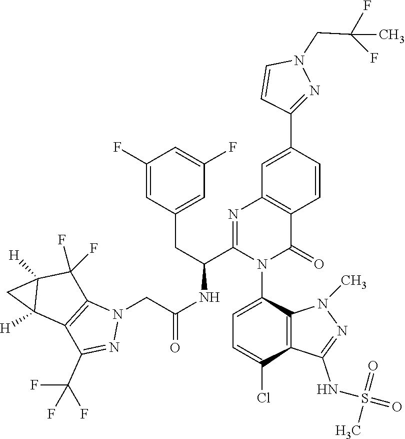

The title compound was prepared according to General Procedure N using 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetic acid as the coupling partner. The experiment afforded the title compound, N—((S)-1-((3P)-3-(4-chloro-1-methyl-3-(methylsulfonamido)-1H-indazol-7-yl)-7-(1-(2,2-difluoropropyl)-1H-pyrazol-3-yl)-4-oxo-3,4-dihydroquinazolin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide. The sample was analyzed using LCMS Method H: retention time=1.51 min.; observed ion=967.2 (M+H). 1H NMR (500 MHz, METHANOL-d4) δ ppm 8.25-8.33 (m, 2H), 8.11 (dd, J=8.34, 1.49 Hz, 1H), 7.82 (d, J=2.38 Hz, 1H), 7.29 (d, J=8.05 Hz, 1H), 7.17 (d, J=7.75 Hz, 1H), 6.95 (d, J=2.38 Hz, 1H), 6.73-6.80 (m, 1H), 6.60 (dd, J=8.05, 2.09 Hz, 2H), 4.82 (dd, J=5.81, 3.43 Hz, 1H), 4.69 (t, J=12.67 Hz, 2H), 4.52-4.63 (m, 2H), 3.61 (s, 3H), 3.48 (dd, J=14.01, 5.07 Hz, 1H), 3.24 (s, 3H), 3.10 (dd, J=14.01, 9.24 Hz, 1H), 2.41-2.50 (m, 2H), 1.68 (t, J=18.78 Hz, 3H), 1.33-1.40 (m, 1H), 1.00-1.07 (m, 1H).

### Preparation of Example 68: N—((S)-1-((3P)-3-(4-chloro-1-methyl-3-(methylsulfonamido)-1H-indazol-7-yl)-7-(1-(2,2-difluoropropyl)-5-methyl-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

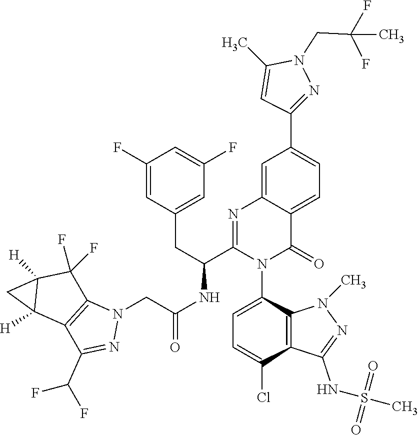

The title compound was prepared according to General Procedure M using 2,2-difluoropropyl trifluoromethanesulfonate as the coupling partner with the following modification: the pyrazole was N—((S)-1-((3P)-3-(4-chloro-3-(N-(4-methoxybenzyl)methylsulfonamido)-1-methyl-1H-indazol-7-yl)-7-(5-methyl-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The experiment afforded the title compound, N—((S)-1-((3P)-3-(4-chloro-1-methyl-3-(methylsulfonamido)-1H-indazol-7-yl)-7-(1-(2,2-difluoropropyl)-5-methyl-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=3.38 min.; observed ion=963.05 (M+H). 1H NMR (500 MHz, METHANOL-d4) δ ppm 8.24-8.31 (m, 2H), 8.06 (dd, J=8.20, 1.64 Hz, 1H), 7.15-7.31 (m, 2H), 6.56-6.80 (m, 5H), 4.82-4.85 (m, 1H), 4.61 (t, J=12.52 Hz, 2H), 4.51 (d, J=1.19 Hz, 2H), 3.61 (s, 3H), 3.46 (dd, J=13.86, 5.22 Hz, 1H), 3.23 (s, 3H), 3.08 (dd, J=14.01, 9.24 Hz, 1H), 2.43 (s, 3H), 2.38-2.42 (m, 1H), 1.72 (t, J=18.93 Hz, 3H), 1.31-1.37 (m, 1H), 0.95-1.02 (m, 1H).

### Preparation of Example 69: N—((S)-1-((3P)-3-(4-chloro-3-(cyclopropanesulfonamido)-1-(2,2-difluoroethyl)-1H-indazol-7-yl)-7-(1-(2,2-difluoropropyl)-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

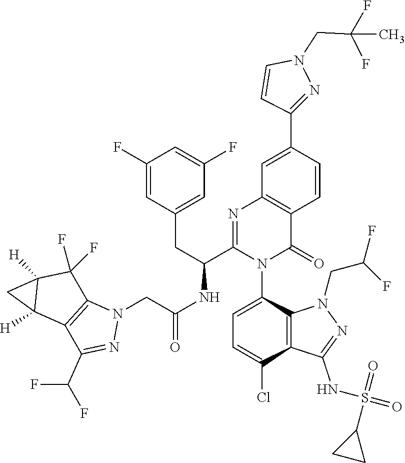

To a mixture of N—((S)-1-((3P)-7-bromo-3-(4-chloro-3-(cyclopropanesulfonamido)-1-(2,2-difluoroethyl)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (30 mg, 0.031 mmol), 1-(2,2-difluoropropyl)-3-(tributylstannyl)-1H-pyrazole (20.40 mg, 0.047 mmol) and copper(I) iodide (0.595 mg, 3.12 μmol) in N,N-Dimethylformamide (DMF) (1.5 mL) was added tetrakis(triphenylphosphine)palladium(0) (3.61 mg, 3.12 μmol). The mixture was degassed (brief high vacuum, then refilled with Ar) and then heated at 100° C. for 3 h. The mixture was cooled to room temperature; filtered; and the filtrate was directly subjected to HPLC purification to afford the title compound, N—((S)-1-((3P)-3-(4-chloro-3-(cyclopropanesulfonamido)-1-(2,2-difluoroethyl)-1H-indazol-7-yl)-7-(1-(2,2-difluoropropyl)-1H-pyrazol-3-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method H: retention time=1.51 min.; observed ion=1025.3 (M+H). 1H NMR (500 MHz, METHANOL-d4) δ ppm 8.26-8.32 (m, 2H), 8.06-8.14 (m, 1H), 7.83 (d, J=2.38 Hz, 1H), 7.37 (d, J=8.05 Hz, 1H), 7.25 (d, J=8.05 Hz, 1H), 6.96 (d, J=2.38 Hz, 1H), 6.50-6.81 (m, 4H), 5.88-6.14 (m, 1H), 4.66-4.75 (m, 3H), 4.55-4.65 (m, 2H), 4.31-4.43 (m, 1H), 3.85-4.00 (m, 1H), 3.40 (dd, J=14.16, 4.92 Hz, 1H), 3.05 (dd, J=14.16, 9.39 Hz, 1H), 2.84-2.93 (m, 1H), 2.33-2.47 (m, 2H), 1.68 (t, J=18.78 Hz, 3H), 1.31-1.37 (m, 1H), 1.05-1.10 (m, 2H), 0.93-1.01 (m, 3H).

### IUPAC Chemical Names:

The IUPAC chemical names for each example are listed below. At this time these names are not recognized by common software such tools such as ChemDraw or JChem. Therefore, the chemical names used throughout the Examples section above were generated with ChemDraw with P/M nomenclature manually inserted. The chemical names can be converted to chemical structures using ChemDraw after the P/M nomenclature—e.g., “(3P)-”—is removed.

| Example | IUPAC Name |
|---|---|
| Example 1 | N-[(1S)-1-[(3P)-3-(4-chloro-3-methanesulfonamido-1- |
| | methyl-1H-indazol-7-yl)-4-oxo-7-[2-(trifluoromethyl)-1,3- |
| | thiazol-4-yl]-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 2 | N-[(1S)-1-[(3P)-3-(4-chloro-3-methanesulfonamido-1- |
| | methyl-1H-indazol-7-yl)-7-(4-methanesulfonylphenyl)-4- |
| | oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 3 | 2-[(3P)-3-(4-chloro-3-methanesulfonamido-1-methyl-1H- |
| | indazol-7-yl)-2-[(1S)-1-{2-[(2S,4R)-9-(difluoromethyl)-5,5- |
| | difluoro-7,8-diazatricyclo[4.3.0.0²,⁴]nona-1(6),8-dien-7- |
| | yl]acetamido}-2-(3,5-difluorophenyl)ethyl]-4-oxo-3,4- |
| | dihydroquinazolin-7-yl]benzoic acid |
| Example 5 | N-[(1S)-1-[(3P)-3-(4-chloro-3-methanesulfonamido-1- |
| | methyl-1H-indazol-7-yl)-7-(2-methoxypyrimidin-4-yl)-4- |
| | oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 6 | N-[(1S)-1-[(3P)-3-(4-chloro-3-methanesulfonamido-1- |
| | methyl-1H-indazol-7-yl)-7-(2,6-difluoropyridin-3-yl)-4- |
| | oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 7 | N-[(1S)-1-[(3P)-3-(4-chloro-3-methanesulfonamido-1- |
| | methyl-1H-indazol-7-yl)-7-(3,5-difluoropyridin-2-yl)-4- |
| | oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 8 | N-[(1S)-1-[(3P)-7-(5-chloro-3-fluoropyridin-2-yl)-3-(4- |
| | chloro-3-methanesulfonamido-1-methyl-1H-indazol-7-yl)- |
| | 4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 9 | N-[(1S)-1-[(3P)-3-(4-chloro-3-methanesulfonamido-1- |
| | methyl-1H-indazol-7-yl)-7-(2-methanesulfonylpyrimidin-4- |
| | yl)-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 10 | N-[(1S)-1-[(3P)-3-(4-chloro-3-methanesulfonamido-1- |
| | methyl-1H-indazol-7-yl)-7-(3-chloro-5-fluoropyridin-2-yl)- |
| | 4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 11 | N-[(1S)-1-[(3P)-3-(4-chloro-3-methanesulfonamido-1- |
| | methyl-1H-indazol-7-yl)-7-(2-cyanopyrimidin-4-yl)-4-oxo- |
| | 3,4-dihydroquinazolin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 12 | N-[(1S)-1-[(3P)-3-(4-chloro-3-methanesulfonamido-1- |
| | methyl-1H-indazol-7-yl)-7-(4-cyano-6-methylpyrimidin-2- |
| | yl)-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 16 | N-[(1S)-1-[(3P)-3-(4-chloro-3-methanesulfonamido-1- |
| | methyl-1H-indazol-7-yl)-7-(2-methoxy-6-methylpyrimidin- |
| | 4-yl)-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 17 | N-[(1S)-1-[(3P)-3-(4-chloro-3-methanesulfonamido-1- |
| | methyl-1H-indazol-7-yl)-7-[5-(difluoromethyl)pyridin-3- |
| | yl]-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 18 | N-[(1S)-1-[(3P)-3-(4-chloro-3-methanesulfonamido-1- |
| | methyl-1H-indazol-7-yl)-7-[3-(difluoromethyl)-2- |
| | fluorophenyl]-4-oxo-3,4-dihydroquinazolin-2-yl]-2- |
| | (3,5-difluorophenyl)ethyl]-2-[(2S,4R)-9-(difluoromethyl)- |
| | 5,5-difluoro-7,8-diazatricyclo[4.3.0.0²,⁴]nona-1(6),8-dien-7- |
| | yl]acetamide |
| Example 19 | N-[(1S)-1-[(3P)-3-(4-chloro-3-methanesulfonamido-1- |
| | methyl-1H-indazol-7-yl)-7-[2-(methoxymethyl)phenyl]-4- |
| | oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 20 | N-[(1S)-1-[(3P)-3-(4-chloro-3-methanesulfonamido-1- |
| | methyl-1H-indazol-7-yl)-7-(1-methyl-1H-pyrazol-5-yl)-4- |
| | oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 21 | N-[(1S)-1-[(3P)-3-(4-chloro-3-methanesulfonamido-1- |
| | methyl-1H-indazol-7-yl)-7-[3-fluoro-6- |
| | (trifluoromethyl)pyridin-2-yl]-4-oxo-3,4-dihydroquinazolin- |
| | 2-yl]-2-(3,5-difluorophenyl)ethyl]-2-[(2S,4R)-9- |
| | (difluoromethyl)-5,5-difluoro-7,8- |
| | diazatricyclo[4.3.0.0²,⁴]nona-1(6),8-dien-7-yl]acetamide |
| Example 22 | N-[(1S)-1-[(3P)-3-(4-chloro-3-methanesulfonamido-1- |
| | methyl-1H-indazol-7-yl)-4-oxo-7-[6- |
| | (trifluoromethyl)pyrazin-2-yl]-3,4-dihydroquinazolin-2-yl]- |
| | 2-(3,5-difluorophenyl)ethyl]-2-[(2S,4R)-9-(difluoromethyl)- |
| | 5,5-difluoro-7,8-diazatricyclo[4.3.0.0²,⁴]nona-1(6),8-dien-7- |
| | yl]acetamide |
| Example 23 | N-[(1S)-1-[(3P)-3-(4-chloro-3-methanesulfonamido-1- |
| | methyl-1H-indazol-7-yl)-4-oxo-7-[3- |
| | (trifluoromethyl)pyrazin-2-yl]-3,4-dihydroquinazolin-2-yl]- |
| | 2-(3,5-difluorophenyl)ethyl]-2-[(2S,4R)-9-(difluoromethyl)- |
| | 5,5-difluoro-7,8-diazatricyclo[4.3.0.0²,⁴]nona-1(6),8-dien-7- |
| | yl]acetamide |
| Example 24 | N-[(1S)-1-[(3P)-3-(4-chloro-3-methanesulfonamido-1- |
| | methyl-1H-indazol-7-yl)-7-(5-fluoropyrimidin-2-yl)-4-oxo- |
| | 3,4-dihydroquinazolin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 25 | N-[(1S)-1-[(3P)-3-(4-chloro-3-methanesulfonamido-1- |
| | methyl-1H-indazol-7-yl)-4-oxo-7-[6- |
| | (trifluoromethyl)pyrimidin-4-yl]-3,4-dihydroquinazolin-2- |
| | yl]-2-(3,5-difluorophenyl)ethyl]-2-[(2S,4R)-9- |
| | (difluoromethyl)-5,5-difluoro-7,8- |
| | diazatricyclo[4.3.0.0²,⁴]nona-1(6),8-dien-7-yl]acetamide |
| Example 26 | N-[(1S)-1-[(3P)-3-(4-chloro-3-methanesulfonamido-1- |
| | methyl-1H-indazol-7-yl)-7-[6-methyl-2- |
| | (trifluoromethyl)pyrimidin-4-yl]-4-oxo-3,4- |
| | dihydroquinazolin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 27 | N-[(1S)-1-[(3P)-3-(4-chloro-3-methanesulfonamido-1- |
| | methyl-1H-indazol-7-yl)-4-oxo-7-[2- |
| | (trifluoromethyl)pyrimidin-4-yl]-3,4-dihydroquinazolin-2- |
| | yl]-2-(3,5-difluorophenyl)ethyl]-2-[(2S,4R)-9- |
| | (difluoromethyl)-5,5-difluoro-7,8- |
| | diazatricyclo[4.3.0.0²,⁴]nona-1(6),8-dien-7-yl]acetamide |
| Example 28 | N-[(1S)-1-[(3P)-3-(4-chloro-3-methanesulfonamido-1- |
| | methyl-1H-indazol-7-yl)-7-[6-(1,1-difluoroethyl)pyridin-2- |
| | yl]-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 29 | N-[(1S)-1-[(3P)-3-(4-chloro-3-methanesulfonamido-1- |
| | methyl-1H-indazol-7-yl)-7-[1-(2,2-dimethylpropyl)-1H- |
| | pyrazol-3-yl]-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 30 | N-[(1S)-1-[(3P)-3-(4-chloro-3-methanesulfonamido-1- |
| | methyl-1H-indazol-7-yl)-7-[5-fluoro-6- |
| | (trifluoromethyl)pyridin-2-yl]-4-oxo-3,4-dihydroquinazolin- |
| | 2-yl]-2-(3,5-difluorophenyl)ethyl]-2-[(2S,4R)-9- |
| | (difluoromethyl)-5,5-difluoro-7,8- |
| | diazatricyclo[4.3.0.0²,⁴]nona-1(6),8-dien-7-yl]acetamide |
| Example 31 | N-[(1S)-1-[(3P)-3-(4-chloro-3-methanesulfonamido-1- |
| | methyl-1H-indazol-7-yl)-7-[1-(cyclopropylmethyl)-1H- |
| | pyrazol-3-yl]-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 32 | N-[(1S)-1-[(3P)-3-(4-chloro-3-methanesulfonamido-1- |
| | methyl-1H-indazol-7-yl)-4-oxo-7-[5-(trifluoromethyl)-1H- |
| | pyrazol-1-yl]-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 34 | N-[(1S)-1-[(3P)-3-(4-chloro-3-methanesulfonamido-1- |
| | methyl-1H-indazol-7-yl)-7-[1-(2,2-difluoroethyl)-1H- |
| | pyrazol-4-yl]-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 35 | N-[(1S)-1-[(3P)-3-(4-chloro-3-methanesulfonamido-1- |
| | methyl-1H-indazol-7-yl)-7-[1-(difluoromethyl)-1H-pyrazol- |
| | 4-yl]-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 36 | N-[(1S)-1-[(3P)-3-(4-chloro-3-methanesulfonamido-1- |
| | methyl-1H-indazol-7-yl)-7-(2-methyl-1,3-benzothiazol-5- |
| | yl)-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 37 | N-[(1S)-1-[(3P)-3-(4-chloro-3-methanesulfonamido-1- |
| | methyl-1H-indazol-7-yl)-7-[4-(1,1-difluoroethyl)pyridin-2- |
| | yl]-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 38 | N-[(1S)-1-[(3P)-3-(4-chloro-3-methanesulfonamido-1- |
| | methyl-1H-indazol-7-yl)-4-oxo-7-[4-(trifluoromethyl)-1H- |
| | pyrazol-1-yl]-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 39 | N-[(1S)-1-[(3P)-7-(4-acetylpyrimidin-2-yl)-3-(4-chloro-3- |
| | methanesulfonamido-1-methyl-1H-indazol-7-yl)-4-oxo-3,4- |
| | dihydroquinazolin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 40 | N-[(1S)-1-[(3P)-3-(4-chloro-3-methanesulfonamido-1- |
| | methyl-1H-indazol-7-yl)-7-(1-cyclopropyl-1H-pyrazol-4- |
| | yl)-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 41 | N-[(1S)-1-[(3P)-3-(4-chloro-3-methanesulfonamido-1- |
| | methyl-1H-indazol-7-yl)-4-oxo-7-[1-(2,2,2-trifluoroethyl)- |
| | 1H-pyrazol-4-yl]-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 42 | N-[(1S)-1-[(3P)-3-(4-chloro-3-methanesulfonamido-1- |
| | methyl-1H-indazol-7-yl)-7-[1-(2-fluoroethyl)-1H-pyrazol-4- |
| | yl]-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 43 | N-[(1S)-1-[(3P)-3-(4-chloro-3-methanesulfonamido-1- |
| | methyl-1H-indazol-7-yl)-7-(1-cyclopropyl-1H-pyrazol-5- |
| | yl)-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 44 | N-[(1S)-1-[(3P)-7-(l,3-benzothiazol-6-yl)-3-(4-chloro-3- |
| | methanesulfonamido-1-methyl-1H-indazol-7-yl)-4-oxo-3,4- |
| | dihydroquinazolin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 45 | N-[(1S)-1-[(3P)-7-(l,3-benzothiazol-5-yl)-3-(4-chloro-3- |
| | methanesulfonamido-1-methyl-1H-indazol-7-yl)-4-oxo-3,4- |
| | dihydroquinazolin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 46 | N-[(1S)-1-[(3P)-7-(l,3-benzoxazol-5-yl)-3-(4-chloro-3- |
| | methanesulfonamido-1-methyl-1H-indazol-7-yl)-4-oxo-3,4- |
| | dihydroquinazolin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 47 | N-[(1S)-1-[(3P)-3-(4-chloro-3-methanesulfonamido-1- |
| | methyl-1H-indazol-7-yl)-4-oxo-7-[1-(2,2,3,3- |
| | tetrafluoropropyl)-1H-pyrazol-3-yl]-3,4-dihydroquinazolin- |
| | 2-yl]-2-(3,5-difluorophenyl)ethyl]-2-[(2S,4R)-9- |
| | (difluoromethyl)-5,5-difluoro-7,8- |
| | diazatricyclo[4.3.0.0²,⁴]nona-1(6),8-dien-7-yl]acetamide |
| Example 48 | N-[(1S)-1-[(3P)-3-(4-chloro-3-methanesulfonamido-1- |
| | methyl-1H-indazol-7-yl)-4-oxo-7-[1-(2,2,3,3,3- |
| | pentafluoropropyl)-1H-pyrazol-3-yl]-3,4- |
| | dihydroquinazolin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 49 | N-[(1S)-1-[(3P)-3-(4-chloro-3-methanesulfonamido-1- |
| | methyl-1H-indazol-7-yl)-7-[1-(2,2-difluorobutyl)-1H- |
| | pyrazol-3-yl]-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 50 | N-[(1S)-1-[(3P)-3-(4-chloro-3-methanesulfonamido-1- |
| | methyl-1H-indazol-7-yl)-4-oxo-7-[1-(3,3,3-trifluoropropyl)- |
| | 1H-pyrazol-3-yl]-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 51 | N-[(1S)-1-[(3P)-3-(4-chloro-3-methanesulfonamido-1- |
| | methyl-1H-indazol-7-yl)-7-[1-(3-fluoropropyl)-1H-pyrazol- |
| | 3-yl]-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 52 | N-[(1S)-1-[(3P)-3-(4-chloro-3-methanesulfonamido-1- |
| | methyl-1H-indazol-7-yl)-7-[1-(3,3-difluorobutyl)-1H- |
| | pyrazol-3-yl]-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 53 | N-[(1S)-1-[(3P)-3-(4-chloro-3-methanesulfonamido-1- |
| | methyl-1H-indazol-7-yl)-4-oxo-7-[1-(4,4,4-trifluorobutyl)- |
| | 1H-pyrazol-3-yl]-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 54 | N-[(1S)-1-[(3P)-3-(4-chloro-3-methanesulfonamido-1- |
| | methyl-1H-indazol-7-yl)-4-oxo-7-{1-[2- |
| | (trifluoromethoxy)ethyl]-1H-pyrazol-3-yl}-3,4- |
| | dihydroquinazolin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 55 | N-[(1S)-1-[(3P)-3-(4-chloro-3-methanesulfonamido-1- |
| | methyl-1H-indazol-7-yl)-7-[2-(fluoromethyl)pyrimidin-4- |
| | yl]-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 56 | N-[(1S)-1-[(3P)-3-(4-chloro-3-methanesulfonamido-1- |
| | methyl-1H-indazol-7-yl)-7-[2-(difluoromethyl)pyrimidin-4- |
| | yl]-4-oxo-3,4-dihydroquinazolin-2-yl]-2-(3,5- |
| | difluorophenyl)ethyl]-2-[(2S,4R)-9-(difluoromethyl)-5,5- |
| | difluoro-7,8-diazatricyclo[4.3.0.0²,⁴]nona-1(6),8-dien-7- |
| | yl]acetamide |
| Example 57 | N-[(1S)-1-[(3P)-3-(4-chloro-3-methanesulfonamido-1- |
| | methyl-1H-indazol-7-yl)-7-[5-methyl-1-(2,2,3,3- |
| | tetrafluoropropyl)-1H-pyrazol-3-yl]-4-oxo-3,4- |
| | dihydroquinazolin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 58 | (2S)-N-[(1S)-1-[(3P)-3-(4-chloro-3-methanesulfonamido-1- |
| | methyl-1H-indazol-7-yl)-7-[1-(2,2-difluoropropyl)-1H- |
| | pyrazol-3-yl]-4-oxo-3,4-dihydroquinazolin-2-yl]-2-(3,5- |
| | difluorophenyl)ethyl]-2-(3-cyclopropyl-1H-pyrazol-1- |
| | yl)propenamide |
| Example 59 | N-[(1S)-1-[(3P)-3-(4-chloro-3-methanesulfonamido-1- |
| | methyl-1H-indazol-7-yl)-4-oxo-7-[1-(2,2,3,3- |
| | tetrafluoropropyl)-1H-pyrazol-3-yl]-3,4-dihydroquinazolin- |
| | 2-yl]-2-(3,5-difluorophenyl)ethyl]-2-[(2S,4R)-5,5-difluoro- |
| | 9-(trifluoromethyl)-7,8-diazatricyclo[4.3.0.0²,⁴]nona-1(6),8- |
| | dien-7-yl]acetamide |
| Example 60 | N-[(1S)-1-[(3P)-3-(4-chloro-3-methanesulfonamido-1- |
| | methyl-1H-indazol-7-yl)-7-[5-methyl-1-(2,2,3,3,3- |
| | pentafluoropropyl)-1H-pyrazol-3-yl]-4-oxo-3,4- |
| | dihydroquinazolin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7- |
| | yl]acetamide |
| Example 61 | N-[(1S)-1-[(3P)-3-(4-chloro-3-methanesulfonamido-1- |
| | methyl-1H-indazol-7-yl)-7-[1-(2,2-difluoropropyl)-1H- |
| | pyrazol-3-yl]-4-oxo-3,4-dihydroquinazolin-2-yl]-2-(3,5- |
| | difluorophenyl)ethyl]-2-(3-cyclopropyl-1H-pyrazol-1- |
| | yl)acetamide |
| Example 62 | N-[(1S)-1-[(3P)-3-[4-chloro-1-(3-fluoropropyl)-3- |
| | methanesulfonamido-1H-indazol-7-yl]-4-oxo-7-[1-(2,2,3,3- |
| | tetrafluoropropyl)-1H-pyrazol-5-yl]-3,4-dihydroquinazolin- |
| | 2-yl]-2-(3,5-difluorophenyl)ethyl]-2-[(2S,4R)-9- |
| | (difluoromethyl)-5,5-difluoro-7,8- |
| | diazatricyclo[4.3.0.0²,⁴]nona-1(6),8-dien-7-yl]acetamide |
| Example 63 | N-[(1S)-1-[(3P)-3-(4-chloro-3-methanesulfonamido-1- |
| | methyl-1H-indazol-7-yl)-4-oxo-7-[1-(2,2,3,3- |
| | tetrafluoropropyl)-1H-pyrazol-3-yl]-3,4-dihydroquinazolin- |
| | 2-yl]-2-(3,5-difluorophenyl)ethyl]-2-(3-cyclopropyl-1H- |
| | pyrazol-1-yl)acetamide |
| Example 64 | N-[(1S)-1-[(3P)-3-[4-chloro-1-(2,2-difluoroethyl)-3- |
| | methanesulfonamido-1H-indazol-7-yl]-7-[1-(2,2- |
| | difluoropropyl)-1H-pyrazol-3-yl]-4-oxo-3,4- |
| | dihydroquinazolin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 65 | (2R)-N-[(1S)-1-[(3P)-3-(4-chloro-3-methanesulfonamido-1- |
| | methyl-1H-indazol-7-yl)-7-[1-(2,2-difluoropropyl)-1H- |
| | pyrazol-3-yl]-4-oxo-3,4-dihydroquinazolin-2-yl]-2-(3,5- |
| | difluorophenyl)ethyl]-2-(3-cyclopropyl-1H-pyrazol-1- |
| | yl)propenamide |
| Example 66 | N-[(1S)-1-[(3P)-3-(4-chloro-3-methanesulfonamido-1- |
| | methyl-1H-indazol-7-yl)-7-[1-(2,2-difluoropropyl)-1H- |
| | pyrazol-3-yl]-4-oxo-3,4-dihydroquinazolin-2-yl]-2-(3,5- |
| | difluorophenyl)ethyl]-2-[(2S,4R)-5,5-difluoro-9- |
| | (trifluoromethyl)-7,8-diazatricyclo[4.3.0.0²,⁴]nona-1(6),8- |
| | dien-7-yl]acetamide |
| Example 67 | N-[(1S)-1-[(3P)-3-[4-chloro-1-(3-fluoropropyl)-3- |
| | methanesulfonamido-1H-indazol-7-yl]-4-oxo-7-[1-(2,2,3,3- |
| | tetrafluoropropyl)-1H-pyrazol-3-yl]-3,4-dihydroquinazolin- |
| | 2-yl]-2-(3,5-difluorophenyl)ethyl]-2-[(2S,4R)-9- |
| | (difluoromethyl)-5,5-difluoro-7,8- |
| | diazatricyclo[4.3.0.0²,⁴]nona-1(6),8-dien-7-yl]acetamide |
| Example 68 | N-[(1S)-1-[(3P)-3-(4-chloro-3-methanesulfonamido-1- |
| | methyl-1H-indazol-7-yl)-7-[1-(2,2-difluoropropyl)-5- |
| | methyl-1H-pyrazol-3-yl]-4-oxo-3,4-dihydroquinazolin-2- |
| | yl]-2-(3,5-difluorophenyl)ethyl]-2-[(2S,4R)-9- |
| | (difluoromethyl)-5,5-difluoro-7,8- |
| | diazatricyclo[4.3.0.0²,⁴]nona-1(6),8-dien-7-yl]acetamide |
| Example 69 | N-[(1S)-1-[(3P)-3-[4-chloro-3-cyclopropanesulfonamido-1- |
| | (2,2-difluoroethyl)-1H-indazol-7-yl]-7-[1-(2,2- |
| | difluoropropyl)-1H-pyrazol-3-yl]-4-oxo-3,4- |
| | dihydroquinazolin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |

### Biological Methods:

HIV cell culture assay—MT=2 cells, 293T cells and the proviral DNA clone of NL₄₋₃ virus were obtained from the NIH AIDS Research and Reference Reagent Program. MT=2 cells were propagated in RPMI 1640 media supplemented with 10% heat inactivated fetal bovine serum (FBS), 100 mg/ml penicillin G and up to 100 units/mL streptomycin. The 293T cells were propagated in DMEM media supplemented with 10% heat inactivated FBS, 100 mg/mL penicillin G and 100 mg/mL streptomycin. A recombinant NL₄₋₃ proviral clone, in which a section of the nef gene was replaced with the Renilla luciferase gene, was used to make the reference virus used in these studies. The recombinant virus was prepared through transfection of the recombinant NL₄₋₃ proviral clone into 293T cells using Transit-293 Transfection Reagent from Mirus Bio LLC (Madison, Wis.). Supernatent was harvested after 2-3 days and the amount of virus present was titered in MT=2 cells using luciferase enzyme activity as a marker by measuring luciferase enzyme activity. Luciferase was quantitated using the EnduRen Live Cell Substrate from Promega (Madison, Wis.). Antiviral activities of compounds toward the recombinant virus were quantified by measuring luciferase activity in MT=2 cells infected for 4-5 days with the recombinant virus in the presence of serial dilutions of the compound.

The 50% effective concentration (EC₅₀) was calculated by using the exponential form of the median effect equation where (Fa)=1/[1+(ED₅₀/drug conc.)m] (Johnson V A, Byington R T. Infectivity Assay. In Techniques in HIV Research. ed. Aldovini A, Walker B D. 71-76. New York: Stockton Press. 1990). The 50% inhibitory concentration (EC₅₀) was calculated by using the exponential form of the median effect equation where percent inhibition=1/[1+(EC₅₀/drug concentration)m], where m is a parameter that reflects the slope of the concentration-response curve.

Compound cytotoxicity and the corresponding CC₅₀ values were determined using the same protocol as described in the antiviral assay except that uninfected cells were used. Cytotoxicity was assessed on day 4 in uninfected MT2 cells by using an XTT (2,3-bis[2-Methoxy-4-nitro-5-sulfophenyl]-2H-tetrazolium-5-carboxyanilide inner salt)-based colorimetric assay (Sigma-Aldrich, St Louis, Mo.).

| Example | EC₅₀ nM | CC₅₀ μM |
|---|---|---|
| Example 1 | 0.091 | >0.1 |
| Example 5 | 0.083 | >0.5 |
| Example 6 | 0.047 | |
| Example 7 | 0.17 | >0.5 |
| Example 8 | 0.3 | >0.5 |
| Example 9 | 0.15 | >0.5 |
| Example 10 | 0.11 | |
| Example 11 | 0.089 | >0.5 |
| Example 12 | 0.087 | >0.5 |
| Example 16 | 0.11 | >0.5 |
| Example 17 | 0.078 | >0.5 |
| Example 18 | 0.54 | >0.5 |
| Example 19 | 0.16 | >0.5 |
| Example 20 | 0.056 | >0.5 |
| Example 21 | 0.15 | >0.1 |
| Example 22 | 0.06 | >0.1 |
| Example 23 | 0.071 | >0.1 |
| Example 24 | 0.045 | >0.1 |
| Example 25 | 0.083 | >0.1 |
| Example 26 | 0.087 | >0.1 |
| Example 27 | 0.066 | >0.1 |
| Example 28 | 0.12 | >0.1 |
| Example 29 | 0.21 | >0.5 |
| Example 30 | 0.22 | >0.5 |
| Example 31 | 0.11 | >0.5 |
| Example 32 | 0.079 | >0.1 |
| Example 34 | 0.054 | >0.1 |
| Example 35 | 0.092 | >0.1 |
| Example 36 | 1.8 | >0.1 |
| Example 37 | 0.33 | >0.1 |
| Example 38 | 0.17 | >0.1 |
| Example 40 | 0.082 | >0.1 |
| Example 41 | 0.097 | >0.1 |
| Example 42 | 0.068 | >0.1 |
| Example 43 | 0.13 | >0.1 |
| Example 44 | 0.61 | >0.1 |
| Example 45 | 0.44 | >0.1 |
| Example 46 | 0.34 | >0.1 |
| Example 47 | 0.070 | >0.1 |
| Example 48 | 0.091 | >0.1 |
| Example 49 | 0.081 | >0.1 |
| Example 50 | 0.066 | >0.1 |
| Example 51 | 0.042 | >0.1 |
| Example 52 | 0.068 | >0.1 |
| Example 53 | 0.10 | >0.1 |
| Example 54 | 0.098 | >0.1 |
| Example 55 | 0.056 | >0.1 |
| Example 56 | 0.017 | >0.1 |
| Example 57 | 0.13 | >0.1 |
| Example 58 | 0.046 | >0.1 |
| Example 59 | 0.11 | >0.1 |
| Example 60 | 0.21 | >0.1 |
| Example 61 | 0.057 | >0.1 |
| Example 62 | 0.074 | >0.1 |
| Example 63 | 0.043 | >0.1 |
| Example 64 | 0.087 | >0.1 |
| Example 65 | 0.17 | >0.1 |
| Example 66 | 0.069 | >0.1 |
| Example 67 | 0.049 | >0.1 |
| Example 68 | 0.096 | >0.1 |
| Example 69 | 0.16 | >0.1 |

The disclosure is not limited to the foregoing illustrative examples and the examples should be considered in all respects as illustrative and not restrictive, reference being made to the appended claims, rather than to the foregoing examples, and all changes which come within the meaning and range of equivalency of the claims are therefore intended to be embraced.

1. A compound of Formula I, or a pharmaceutically acceptable salt thereof: 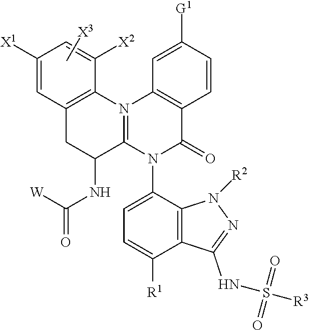
wherein:
X¹ and X² are independently selected from H, F, Cl or —CH₃ and X³ is H, F, Cl, —CH₃, —OCH₃, —OCHF₂, or —OCF₃ with the proviso that within the group X¹, X², and X³ the substituent Cl is not used more than twice and the substituent —CH₃ is not used more than twice;
R¹ is H, Cl, or CH₃;
R² is H, C₁-C₃alkyl optionally substituted with 1-3 fluorines, or C₃-C₆ cycloalkyl optionally substituted with 1-2 fluorines;
R³ is C₁-C₃alkyl or C₃-C₄ cycloalkyl;
G¹ is phenyl substituted once with —CO₂H or —CH₂O(C₁-C₃alkyl) wherein C₁-C₃alkyl is optionally substituted with 1-3 fluorines, or G¹ is fluorophenyl or difluorophenyl substituted once either at the ortho or meta position with C₁-C₂alkyl wherein C₁-C₂alkyl is substituted with 1-3 fluorines, or G¹ is phenyl, pyridine, pyrazine or pyrimidine substituted once with —SF₅, or G is one of the following: 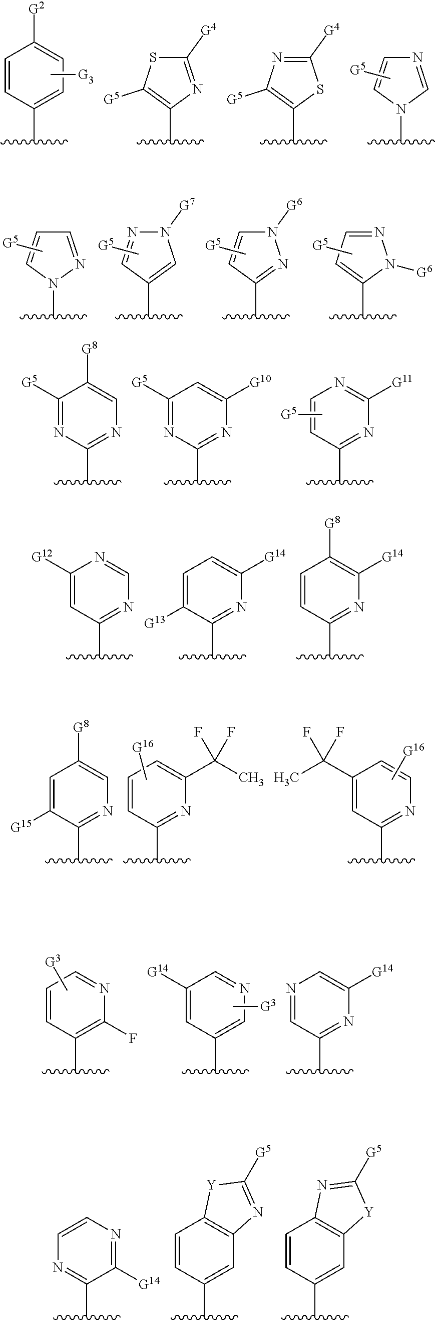
G² is —SO₂(C₁-C₃alkyl);
G³ is H, Cl, or F;
G⁴ is C₁-C₃alkyl substituted with 1-3 fluorines, or G⁴ is —O(C₁-C₃alkyl), —S(O₂)CH₃, or —C(CH₃)₂OH;
G⁵ is H, or methyl optionally substituted with 1-3 fluorines;
G⁶ is cyclopropyl, —CH₂cyclopropyl, C₁-C₃alkyl substituted with 1-5 fluorines, C₄alkyl optionally substituted with 1-5 fluorines, C₅alkyl, or —(C₂-C₃alkyl)O(C₁-C₂alkyl optionally substituted with 1-3 fluorines);
G⁷ is H, C₁-C₃alkyl or G⁶
G⁸ is F, or Cl;
G₉ is H, —O(C₁-C₃alkyl) or C₁-C₃alkyl wherein C₁-C₃alkyl is optionally substituted with 1-3 fluorines;
G¹⁰ is —CN, —COCH₃, —SO₂(C₁-C₃alkyl) or Cl;
G¹¹ is —O(C₁-C₂alkyl optionally substituted with 1-3 fluorines), —SO₂(C₁-C₃alkyl), —CN, —CH₂F, —CHF₂, —CF₃, or —CF₂CH₃;
G¹² is methyl optionally substituted with 1-3 fluorines;
G¹³ is F, —CH₃, —CHF₂, —CF₃, —OCH₃, —SO₂CH₃;
G¹⁴ is C₁-C₂alkyl substituted with 1-3 fluorines;
G¹⁵ is H, F, Cl, —CH₃, —CH₂F, —CHF₂, —CF₃, OCH₃, —SO₂CH₃;
G¹⁶ is F, Cl, or methyl optionally substituted with 1-3 fluorines;
Y is O, S, or N;
W is selected from: 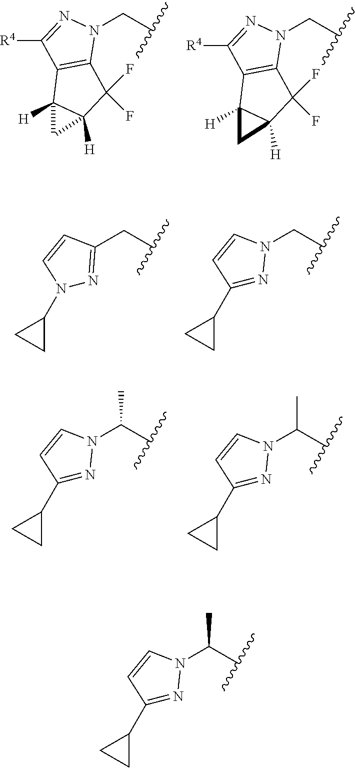
wherein R⁴ is methyl optionally substituted with 1-3 fluorines.

2. A compound or salt according to claim 1 wherein W is the following:

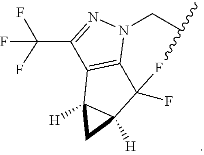

3. A compound or salt according to claim 1 wherein W is the following:

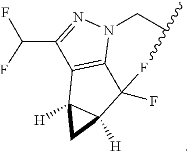

4. A compound or salt according to claim 1 wherein W is the following:

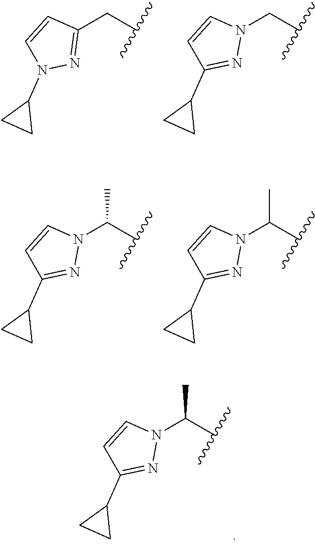

5. A compound or salt according to claim 1 wherein R¹ is Cl; R² is methyl, 2,2-difluoroethyl, or 2,2,2-trifluoroethyl; and R³ is methyl or cyclopropyl.

6. A compound or salt according to claim 1 wherein R¹ is Cl; R² is methyl; and R³ is methyl.

7. A compound or salt according to claim 1 wherein R¹ is Cl; R² is 3-fluoropropyl; and R³ is methyl.

8. A compound or salt according to claim 1 wherein X³ is H.

9. A compound or salt according to claim 1 wherein X¹ is F, X² is F, and X³ is H.

10. A compound or salt according to claim 1 wherein if X³ is H then at least one of X¹ and X² is other than F.

11. A compound or salt according to claim 1 wherein X¹ is H, X² is H and X³ is F.

12. A compound or salt according to claim 1 wherein G¹ is phenyl substituted once with —CO₂H or —CH₂O(C₁-C₃alkyl) wherein C₁-C₃alkyl is optionally substituted with 1-3 fluorines, or G¹ is fluorophenyl or difluorophenyl substituted once either at the ortho or meta position with C₁-C₂alkyl wherein C₁-C₂alkyl is substituted with 1-3 fluorines, or G¹ is the following:

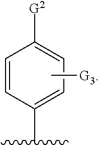

13. A compound or salt according to claim 1 wherein G¹ is one of the following:

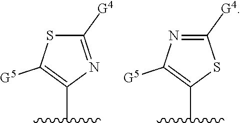

14. A compound or salt according to claim 1 wherein G¹ is one of the following:

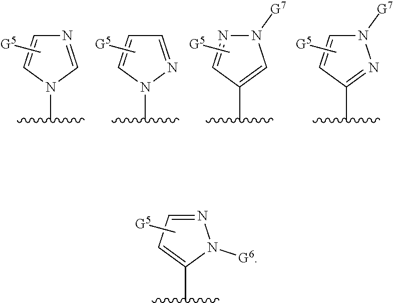

15. A compound or salt according to claim 1 wherein G¹ is one of the following:

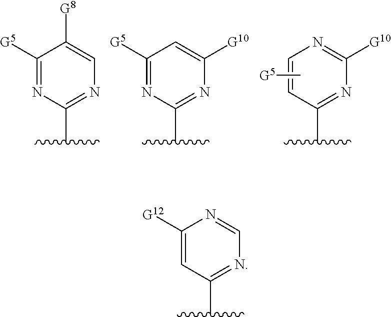

16. A compound or salt according to claim 1 wherein G¹ is one of the following:

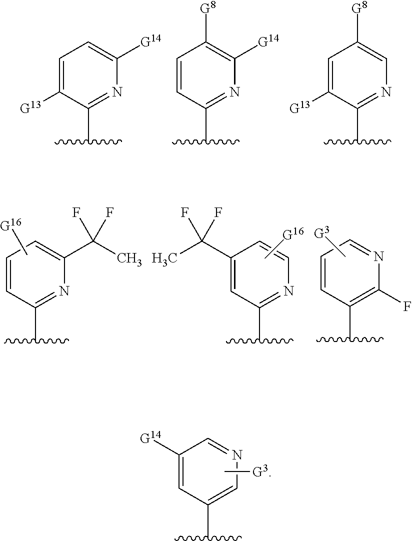

17. A compound or salt according to claim 1 wherein G¹ is one of the following:

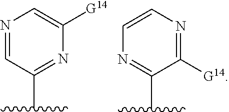

18. A compound or salt according to claim 1 wherein G¹ is one of the following:

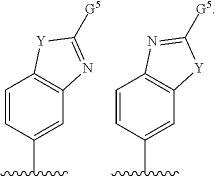

19. A compound or salt according to claim 1 wherein G¹ is one of the following:

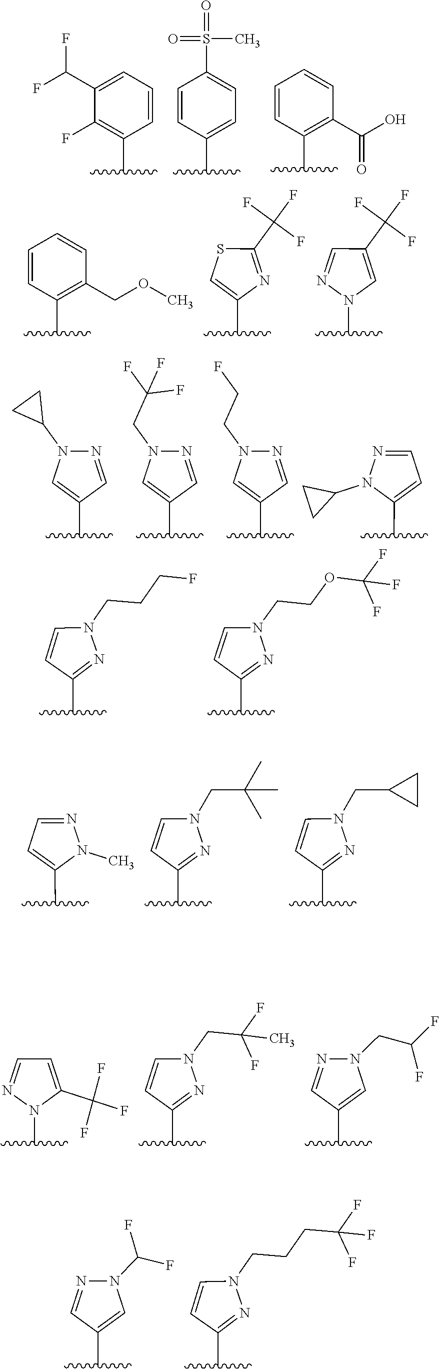

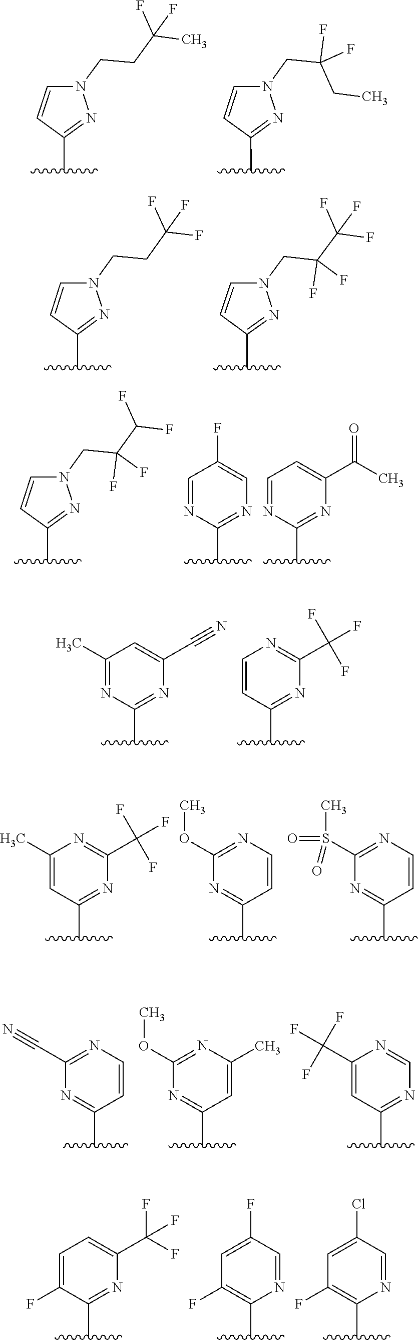

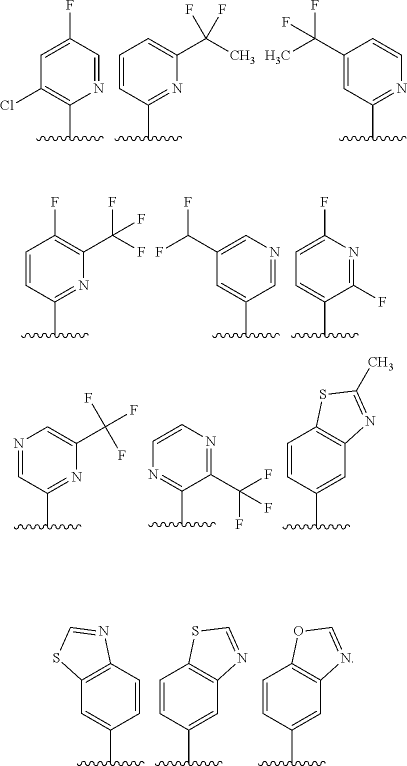

20. A compound or salt according to claim 1 wherein G¹ is one of the following:

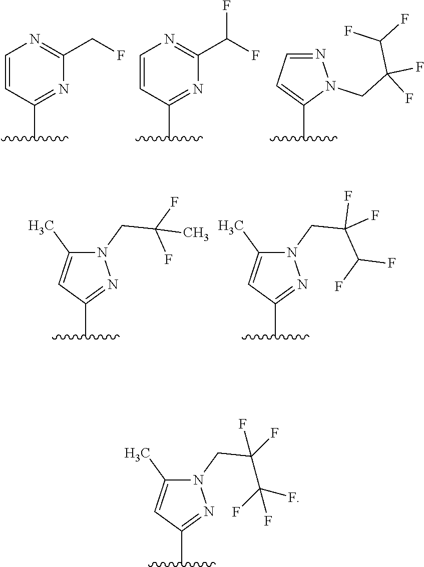

21. A compound or salt according to claim 1 wherein the stereochemistry is as depicted below:

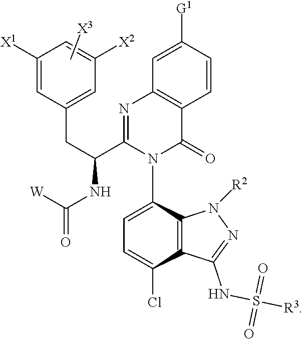

22. A compound or salt according claim 1 wherein the stereochemistry is as depicted below:

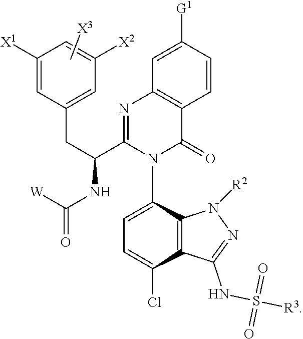

23. A compound or salt according to claim 1, selected from the group consisting of: 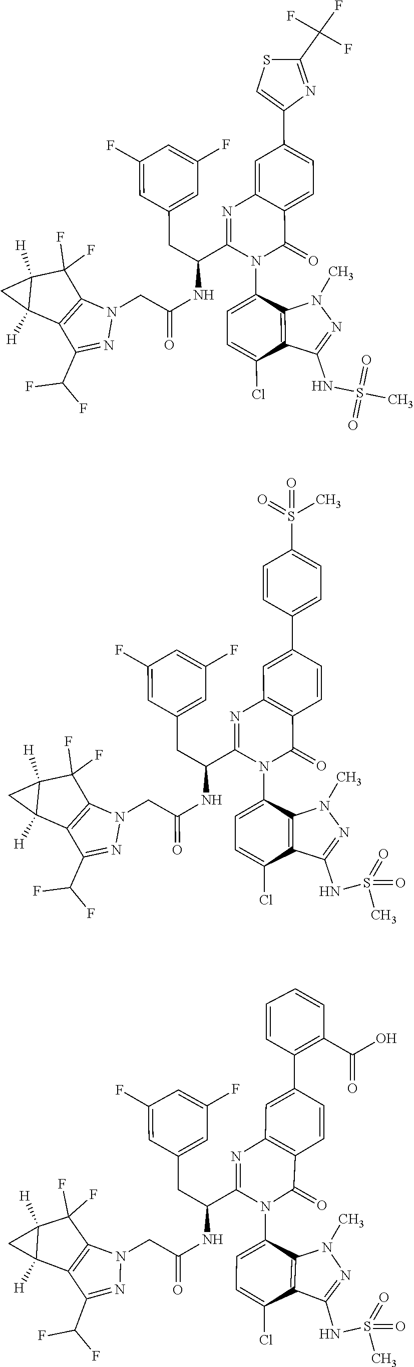 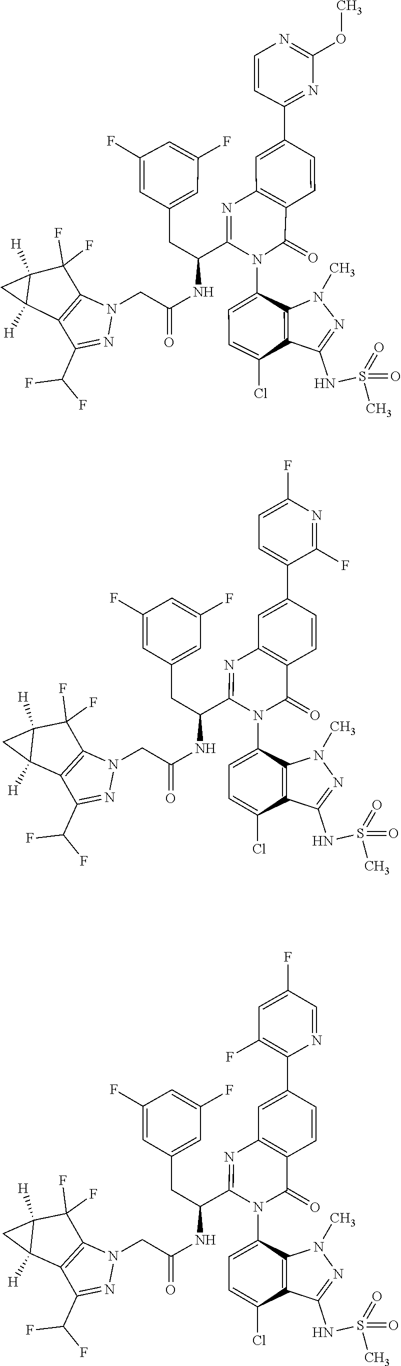 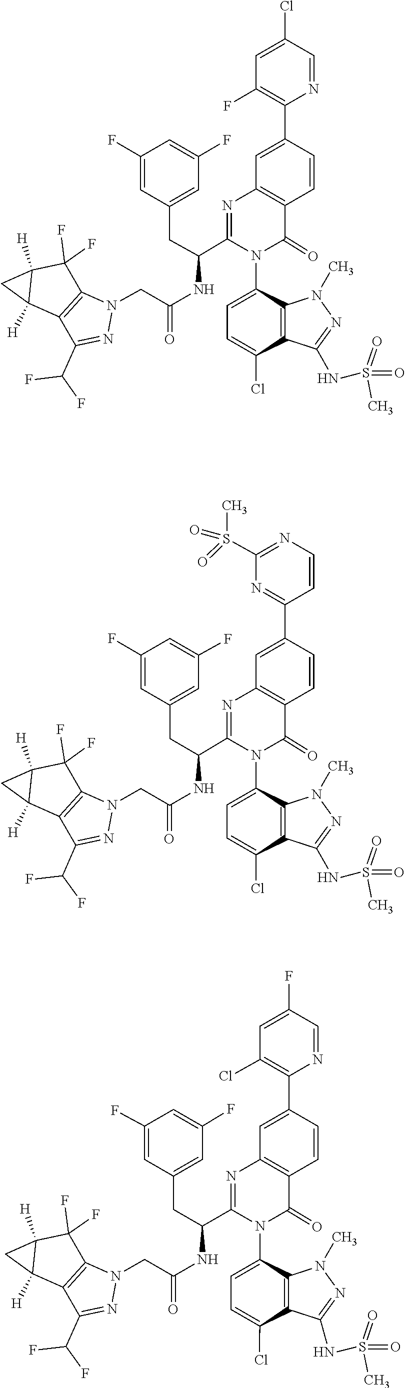 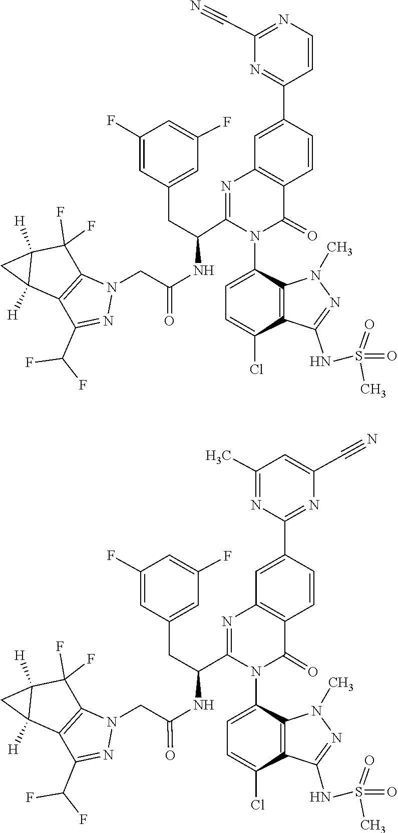 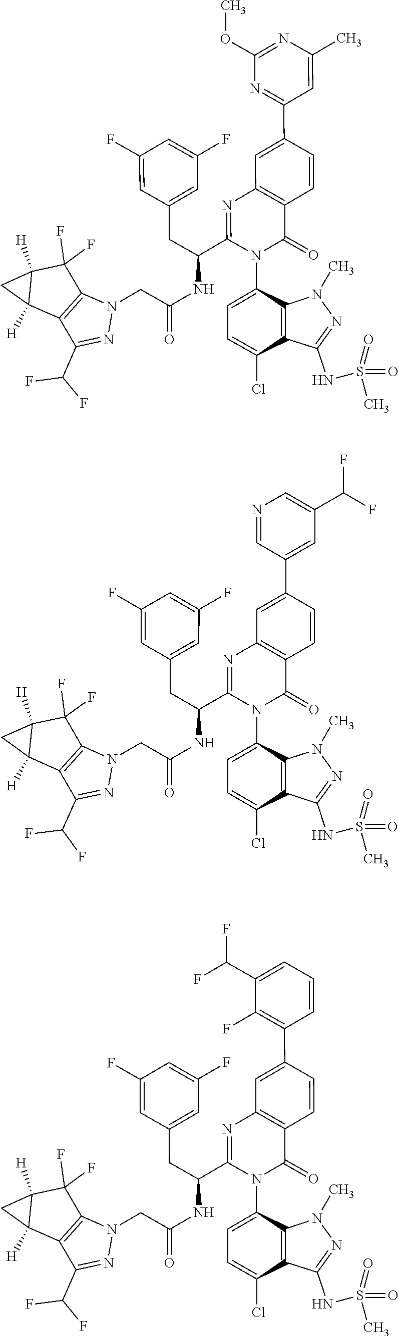
and pharmaceutically acceptable salts thereof.

24. A compound or salt according to claim 1, selected from the group consisting of: 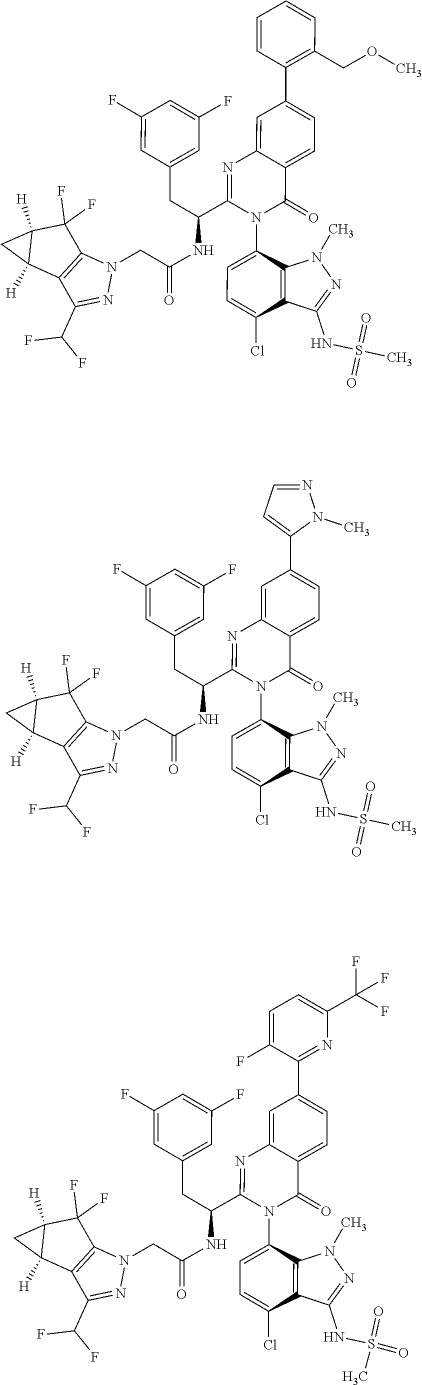 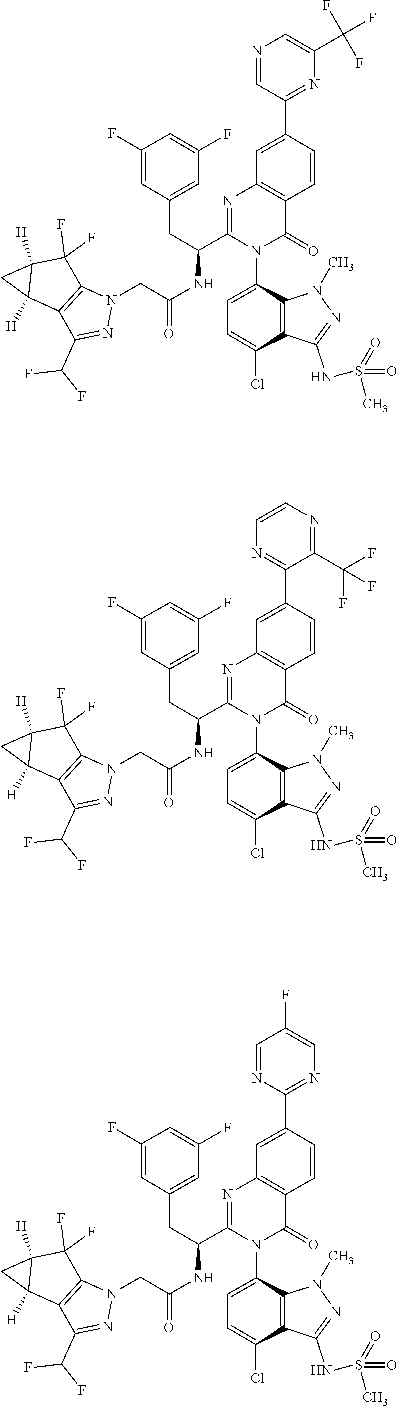 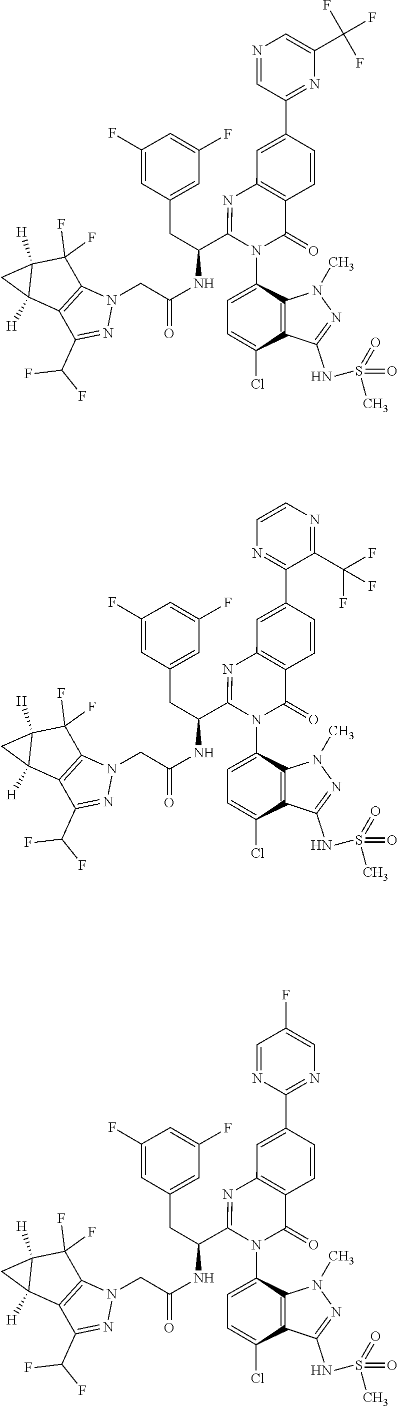 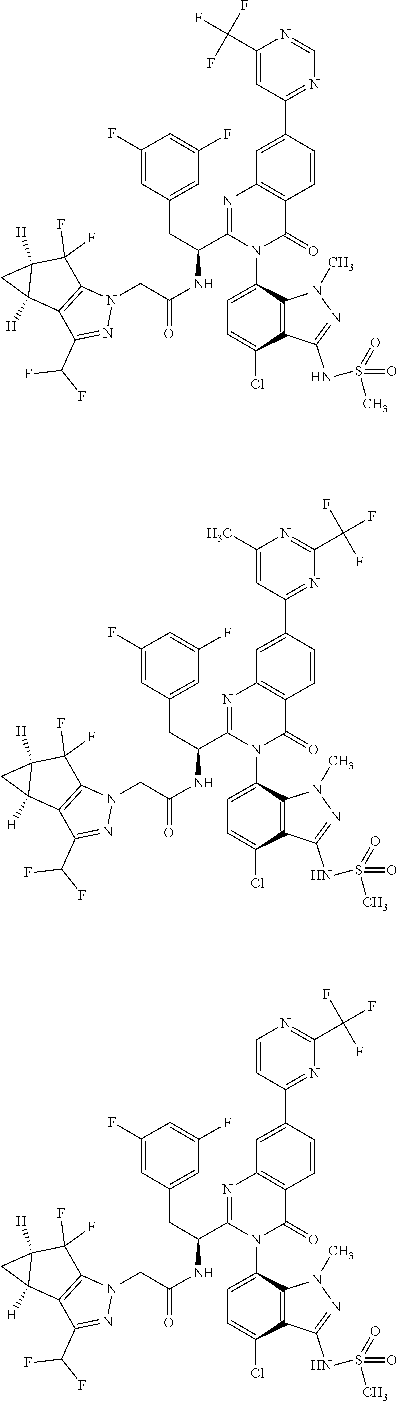 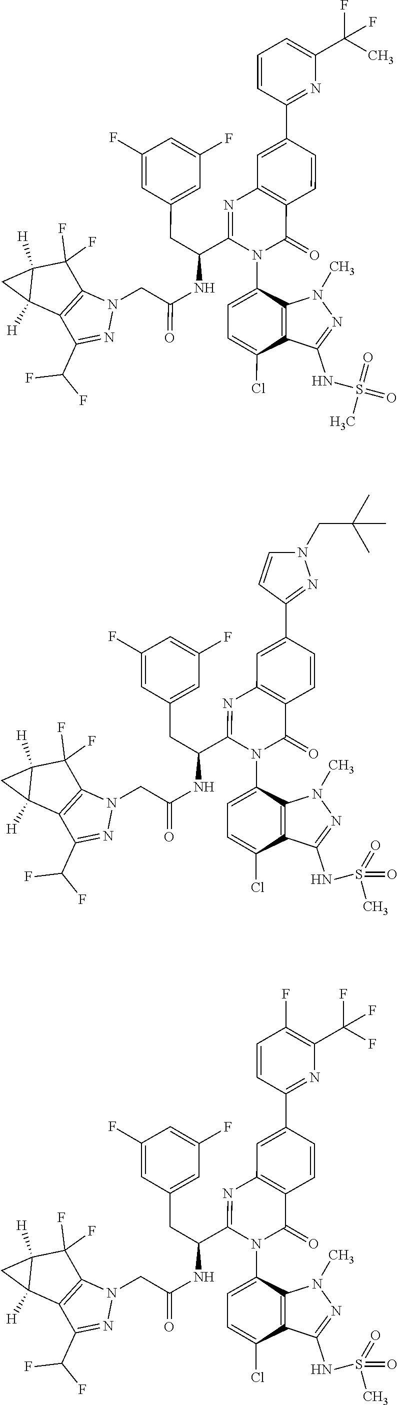 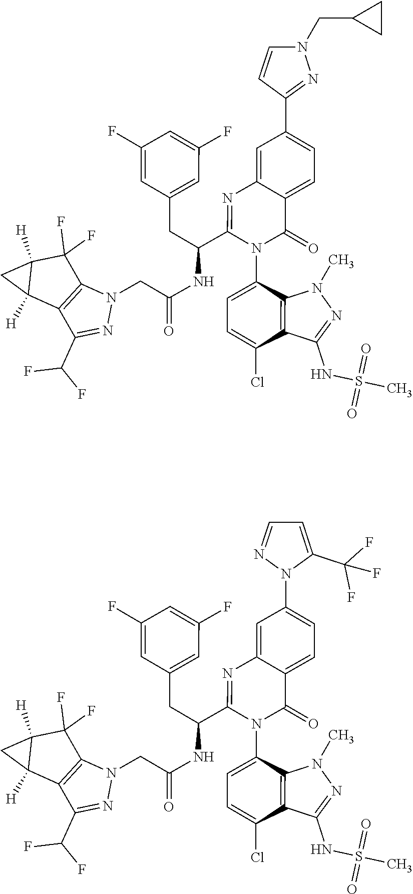 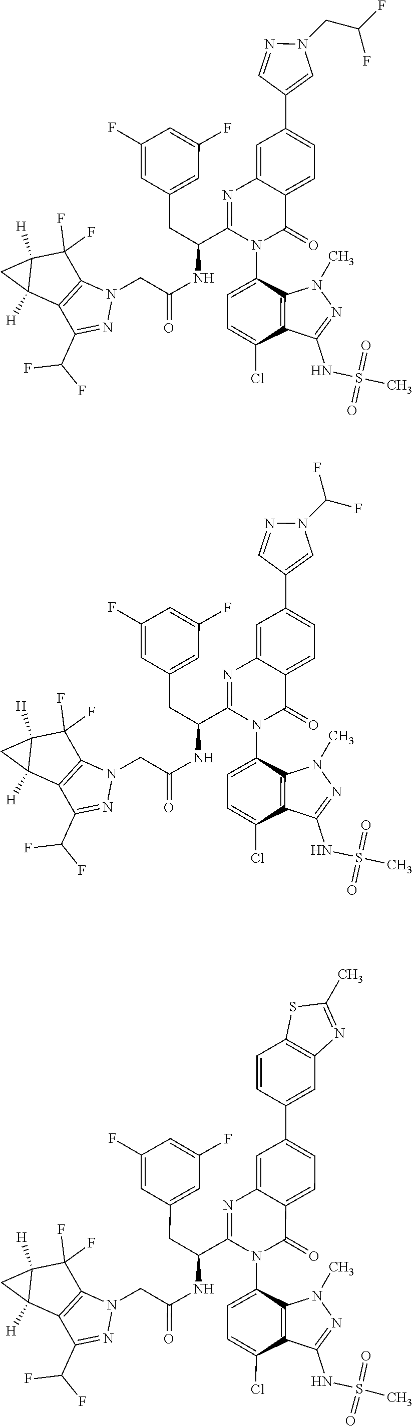
and pharmaceutically acceptable salts thereof.

25. A compound or salt according to claim 1, selected from the group consisting of: 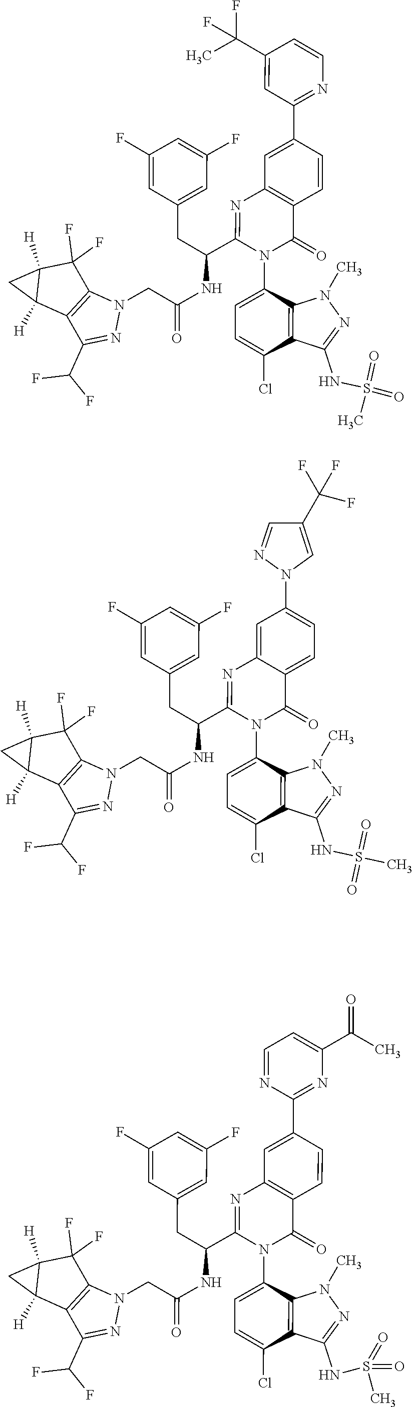 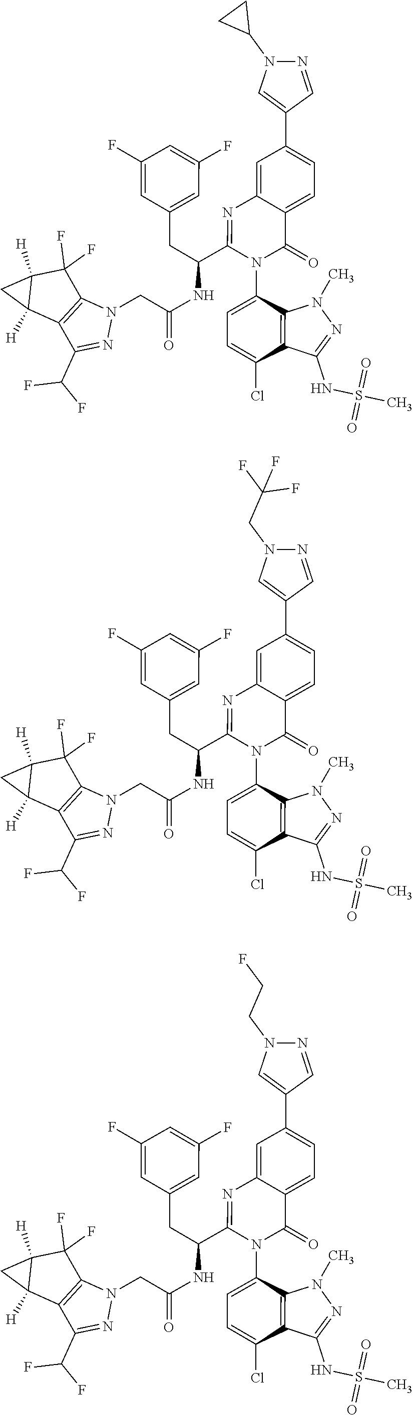 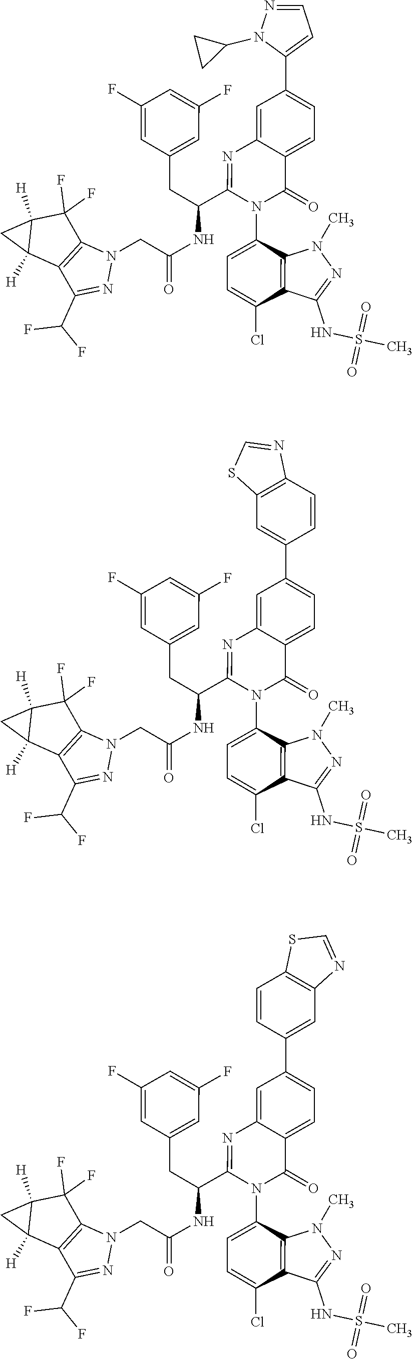 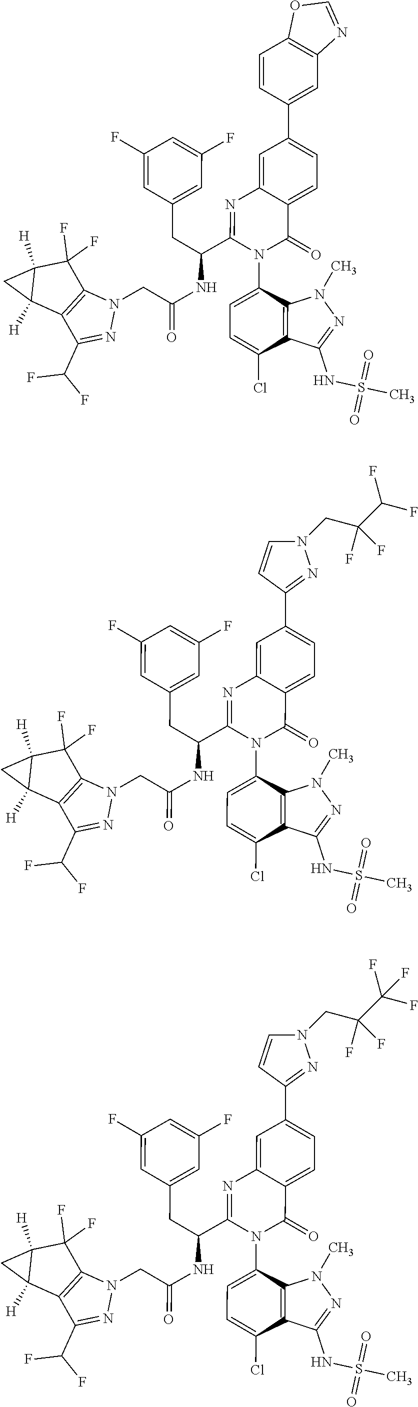 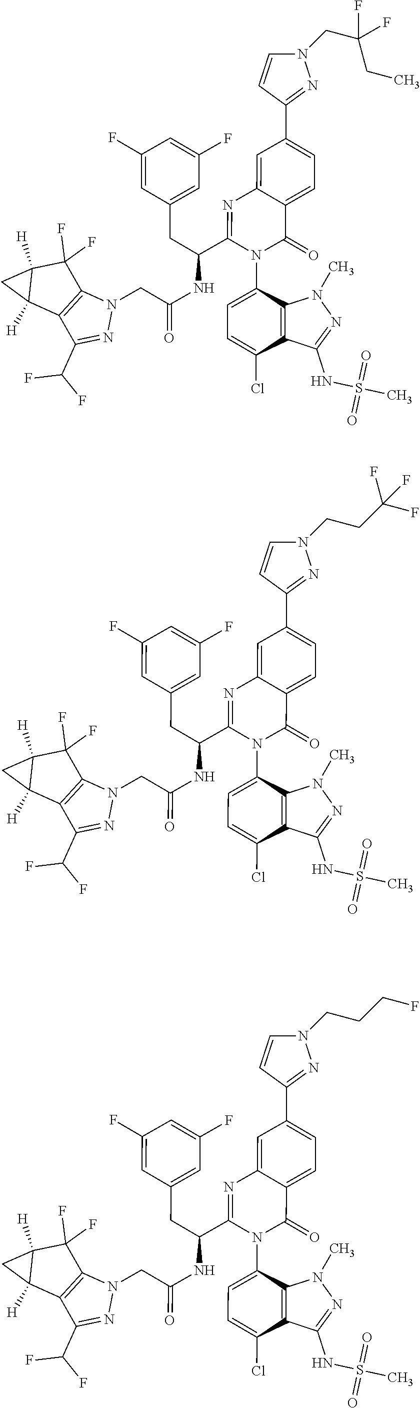 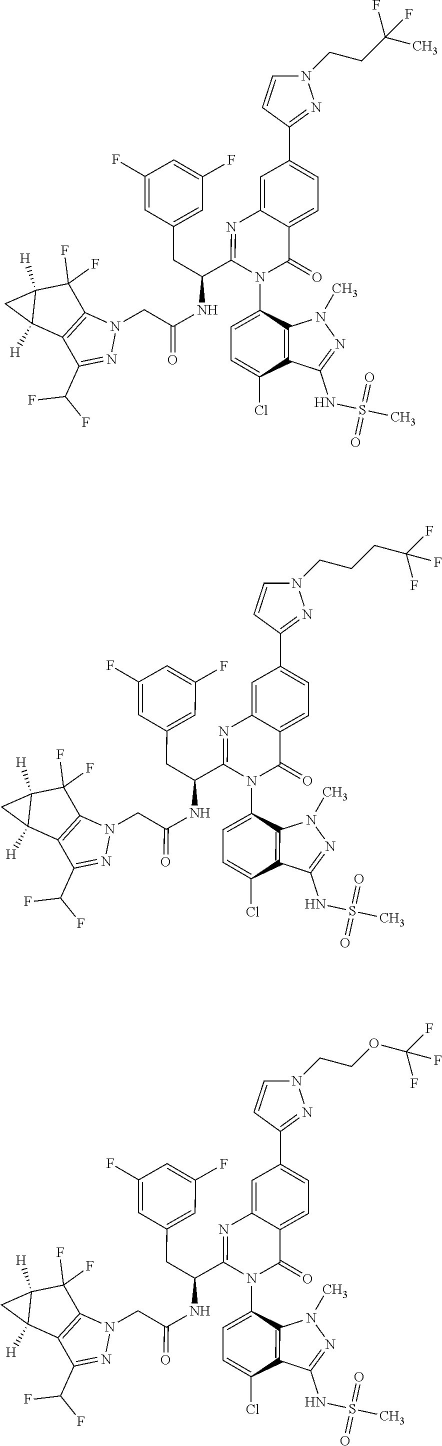
and pharmaceutically acceptable salts thereof.

26. A compound or salt according to claim 1, selected from the group consisting of: 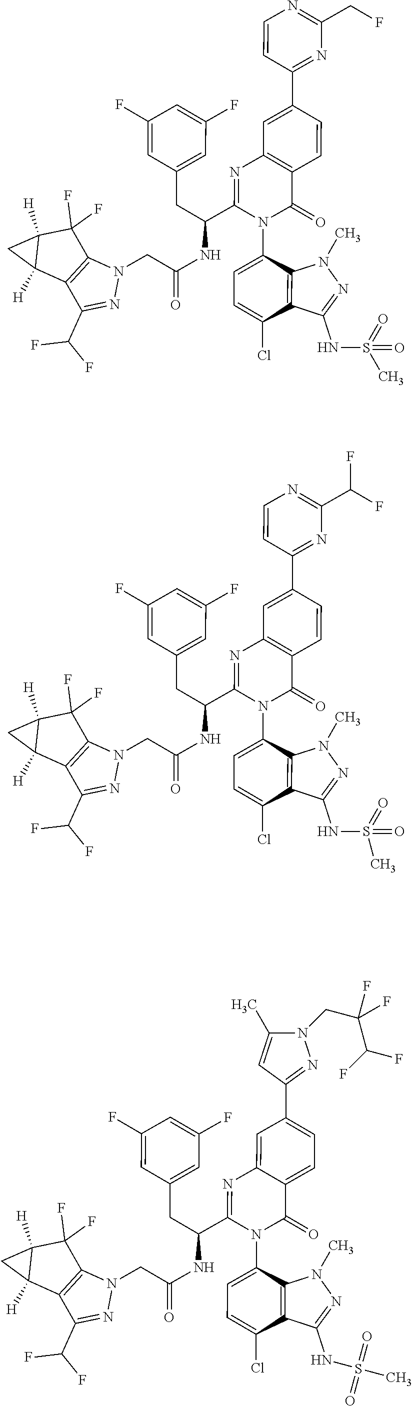 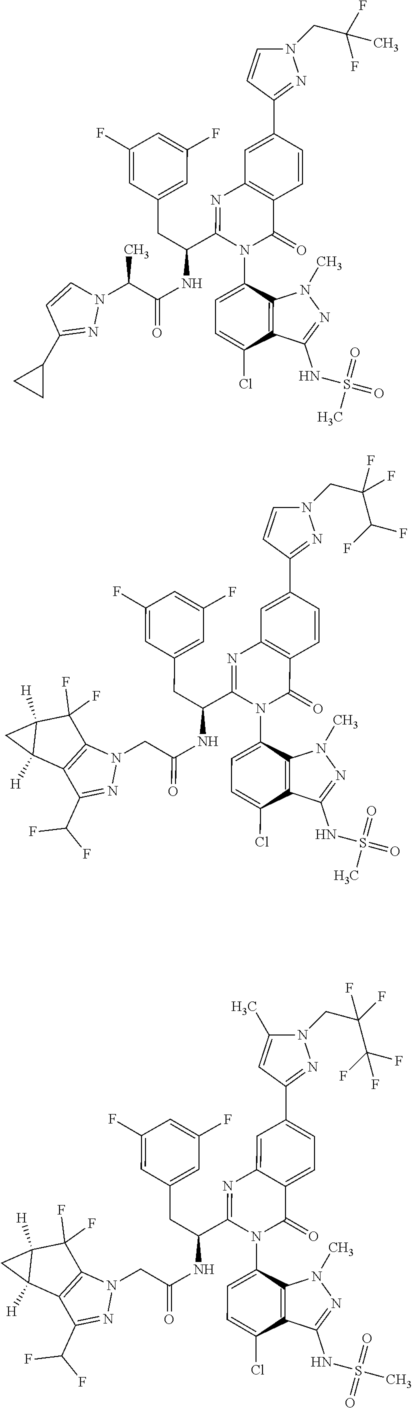 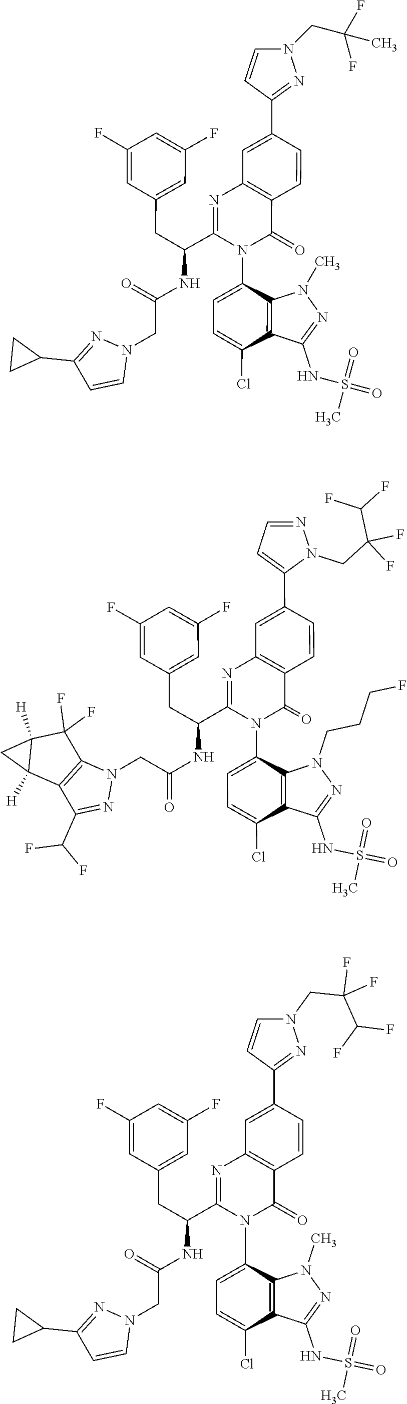 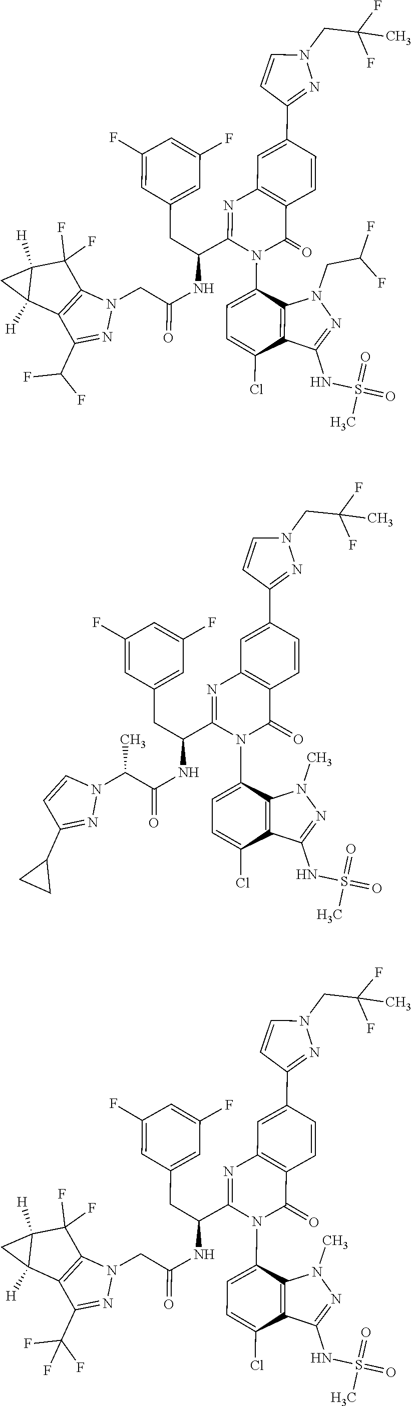 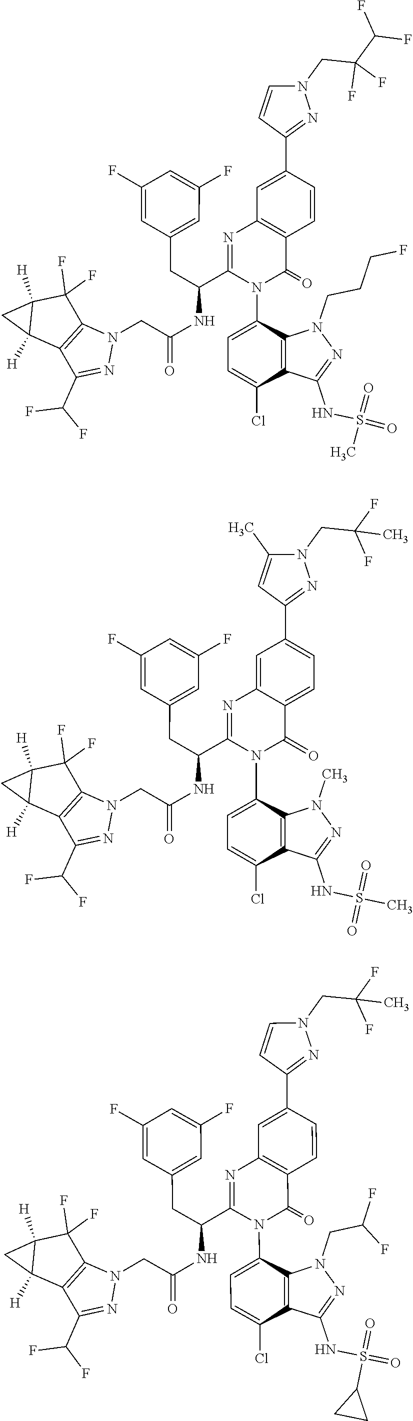
and pharmaceutically acceptable salts thereof.

27. A compound or salt according to claim 1 with the following structure: 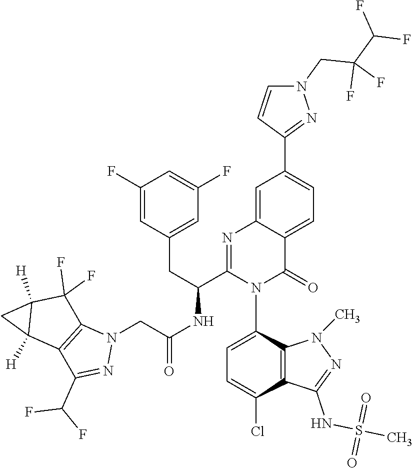
and pharmaceutically acceptable salts thereof.

28. A compound or salt according to claim 1 with the following structure: 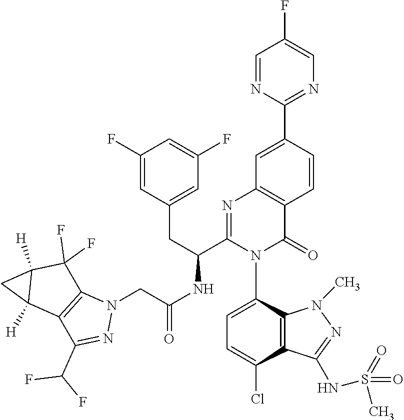
and pharmaceutically acceptable salts thereof.

29. A pharmaceutical composition comprising a compound or salt according to claim 1.

30. A composition according to claim 29 further comprising a pharmaceutically acceptable excipient.

31. A composition according to claim 29 suitable for oral administration, for intramuscular injection, or for subcutaneous injection.

32. A method of treating HIV infection in a human comprising administration of a compound or salt according to claim 1.

33. The method of claim 32 wherein said administration is oral.

34. The method of claim 32 wherein said administration is intramuscular injection or subcutaneous injection.

35. The method of claim 32 wherein said method further comprises administration of at least one other agent used for treatment of HIV infection in a human.

36. The method of claim 35 wherein said at least one other agent is selected from the group consisting of dolutegravir, bictegravir, lamivudine, fostemsavir, and cabotegravir.

37-39. (canceled)
